SEQUENCE LISTING (43 sequences)

SEQ ID NO: 1 - protein, 237 aa, synthetic construct
QVQLQESGPGLVKPSETLSVTCSVSGDSMNNYYWTWIRQSPGKGLEWIGYISDRESATYNPSLNSRVVISRDTSKNQLSLKLNSVTPADTAVYYCATARRGQRIYGVVSFGEFFYYYSMDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDK

SEQ ID NO: 2 - protein, 214 aa, synthetic construct
SYVRPLSVALGETARISCGRQALGSRAVQWYQHRPGQAPILLIYNNQDRPSGIPERFSGTPDINFGTRATLTISGVEAGDEADYYCHMWDSRSGFSWSFGGATRLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 3 - DNA, 19 bp, synthetic construct
  source 1..19: mol_type = other DNA
kkacagtcactgagctgct

SEQ ID NO: 4 - DNA, 19 bp, synthetic construct
  source 1..19: mol_type = other DNA
gtacagtcaccaagctgct

SEQ ID NO: 5 - DNA, 64 bp, synthetic construct
  source 1..64: mol_type = other DNA
agacgtgtgctcttccgatctnntacnnnnnnagtnnnnnngtgtccactcccaagtgcagctg

SEQ ID NO: 6 - DNA, 63 bp, synthetic construct
  source 1..63: mol_type = other DNA
agacgtgtgctcttccgatctnntacnnnnnnagtnnnnnnaggtgcagttggtggagtctgg

SEQ ID NO: 7 - DNA, 33 bp, synthetic construct
  source 1..33: mol_type = other DNA
gtgactggagttcagacgtgtgctcttccgatc

SEQ ID NO: 8 - DNA, 61 bp, synthetic construct
  source 1..61: mol_type = other DNA
  misc_difference 38..45: note = may be absent
acactctttccctacacgacgctcttccgatctnnnnnnnnnnnngggggaagacatttgg

SEQ ID NO: 9 - DNA, 61 bp, synthetic construct
  source 1..61: mol_type = other DNA
  misc_difference 38..45: note = may be absent
acactctttccctacacgacgctcttccgatctnnnnnnnnnnnnagtggatagacmgatg

SEQ ID NO: 10 - DNA, 61 bp, synthetic construct
  source 1..61: mol_type = other DNA
  misc_difference 38..45: note = may be absent
acactctttccctacacgacgctcttccgatctnnnnnnnnnnnnaagggatagacagatg

SEQ ID NO: 11 - DNA, 62 bp, synthetic construct
  source 1..62: mol_type = other DNA
  misc_difference 38..45: note = may be absent
acactctttccctacacgacgctcttccgatctnnnnnnnnnnnntcagtgggtagatggtg

SEQ ID NO: 12 - DNA, 19 bp, synthetic construct
  source 1..19: mol_type = other DNA
actgccatcaatcttccac

SEQ ID NO: 13 - DNA, 64 bp, synthetic construct
  source 1..64: mol_type = other DNA
agacgtgtgctcttccgatctnntacnnnnnnagtnnnnnntgttctaacccaacctccctctg

SEQ ID NO: 14 - DNA, 64 bp, synthetic construct
  source 1..64: mol_type = other DNA
agacgtgtgctcttccgatctnntacnnnnnnagtnnnnnnaaaccaacggttcctatgtcagg

SEQ ID NO: 15 - DNA, 61 bp, synthetic construct
  source 1..61: mol_type = other DNA
  misc_difference 38..45: note = may be absent
acactctttccctacacgacgctcttccgatctnnnnnnnnnnnnggatggtgggaagatg

SEQ ID NO: 16 - protein, 237 aa, synthetic construct
QVQLQESGPGLVKPSETLSVTCSVSGDSMNNYYWTWIRQSPGKGLEWIGYISDRESATYNPSLNSRVVISRDTSKNQLSLKLNSVTPADTAVYYCATARRGQRIYGMVAFGEFFYYYSMDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDK

SEQ ID NO: 17 - protein, 214 aa, synthetic construct; identical to SEQ ID NO: 2

SEQ ID NO: 18 - protein, 237 aa, synthetic construct
QVQLQESGPGLVKPSETLSVTCSVSGDSMNKYYWTWIRQSPGKGLEWIGYISDRESATYNPSLNSRVVISRDTSKNQLSLKLNSVTPADTAVYYCATARRGQRIYGMVAFGEFFYYYSMDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDK

SEQ ID NO: 19 - protein, 237 aa, synthetic construct
QVQLQESGPGLVKPSETLSVTCSVSGDSMNKYYWTWIRQSPGKGLEWIGYISDRDSATYNPSLNSRVVISRDTSKNQLSLKLNSVTPADTAVYYCATARRGQRIYGMVAFGEFFYYYSMDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDK

SEQ ID NO: 20 - protein, 237 aa, synthetic construct; identical to SEQ ID NO: 19

SEQ ID NO: 21 - protein, 214 aa, synthetic construct
SYVRPLSVALGETARISCGRQALGSRAVQWYQHRPGQAPILLIYNNQDRPSGIPERFSGTPDINFGTRATLTISGVEAGDEADYYCHLWDSRSGFSWSFGGATRLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 22 - DNA, 20 bp, synthetic construct
  source 1..20: mol_type = other DNA
ctggatgacttcagtgttgt

SEQ ID NO: 23 - DNA, 19 bp, synthetic construct
  source 1..19: mol_type = other DNA
ccaggtcacattcatcgtg

SEQ ID NO: 24 - protein, 17 aa, Mus musculus
YYAMDYWGQGTSVTVSS

SEQ ID NO: 25 - DNA, 53 bp, Mus musculus
  source 1..53: mol_type = genomic DNA
attactatgctatggactactggggtcaaggaacctcagtcaccgtctcctca

SEQ ID NO: 26 - DNA, 53 bp, Mus musculus
  source 1..53: mol_type = genomic DNA
tgaggagacggtgactgaggttccttgaccccagtagtccatagcatagtaat

SEQ ID NO: 27 - protein, 37 aa, synthetic construct
KDLREDECEEWWSDYYDFGKQLPCAKSRGGLVGIADN

SEQ ID NO: 28 - protein, 37 aa, synthetic construct
KDLRENECEEWWSDYYDFGKELPCAKSRGGLEGIADN

SEQ ID NO: 29 - protein, 37 aa, synthetic construct
KDLRENECEEWWSDYYDFGKELPCARSRGGLVGIADN

SEQ ID NO: 30 - protein, 37 aa, synthetic construct
KDLRENECEEWWSDYYDFGKELPCARSRGGLEGIADN

SEQ ID NO: 31 - protein, 40 aa, synthetic construct
CAKDLREDECEEWWSDYYDFGKQLPCAKSRGGLVGIADNW

SEQ ID NO: 32 - protein, 39 aa, synthetic construct
CAKDLGESENEEWATDYYDFSIGYPGQDPRGVVGAFDIW

SEQ ID NO: 33 - protein, 87 aa, synthetic construct
CVKLTPLCVTLQCTNVTNNITDDMRGELKNCSFNMTTELRDKKQKVYSLFYRLDVVQINENQGNRSNNSNKEYRLINCNTSAITQAC

SEQ ID NO: 34 - protein, 88 aa, synthetic construct
CVKLTPLCVTLECRQVNTTNATSSVNVTNGEEIKNCSFNATTEIRDKKQKVYALFYRLDIVPLEEERKGNSSKYRLINCNTSAITQAC

SEQ ID NO: 35 - protein, 82 aa, synthetic construct
CVKLTPLCVTLDCQAFNSSSHTNSSIAMQEMKNCSFNVTTELRDKKKKEYSFFYKTDIEQINKNGRQYRLINCNTSAITQAC

SEQ ID NO: 36 - protein, 32 aa, synthetic construct
CVREAGGPDYRNGYNYYDFYDGYYNYHYMDVW

SEQ ID NO: 37 - protein, 32 aa, synthetic construct
CAREAGGPIWHDDVKYYDFNDGYYNYHYMDVW

SEQ ID NO: 38 - protein, 10 aa, synthetic construct
YYDFWSGYYT

SEQ ID NO: 39 - protein, 10 aa, synthetic construct
YYDILTGYYN

SEQ ID NO: 40 - protein, 12 aa, synthetic construct
YYDYVWGSYAYT

SEQ ID NO: 41 - protein, 10 aa, synthetic construct
YYYDSSGYYY

SEQ ID NO: 42 - protein, 4 aa, synthetic construct
YYDF

SEQ ID NO: 43 - RNA, 44 nt, synthetic construct
  source 1..44: mol_type = other RNA
taatttctactgtttgtagatcttgaccccagtagtccatagca

(19) United States
(12) Patent Application Publication
(10) Pub. No.: US 2026/0117259 A1
(43) Pub. Date: Apr. 30, 2026

(54) DIRECTED IN VIVO AFFINITY MATURATION OF ANTIBODIES

(71) Applicant: University of Florida Research Foundation, Incorporated, Gainesville, FL (US)

(72) Inventors: Michael R. Farzan, Gainesville, FL (US); Tianling Ou, Gainesville, FL (US); Wenhui He, Gainesville, FL (US); Yiming Yin, Gainesville, FL (US)

(73) Assignee: University of Florida Research Foundation, Incorporated, Gainesville, FL (US)

(21) Appl. No.: 19/116,765

(22) PCT Filed: Sep. 29, 2023
(86) PCT No.: PCT/US2023/075636

§ 371 Date: Mar. 28, 2025

Related U.S. Application Data
(60) Provisional application No. 63/411,510, filed on Sep. 29, 2022.

(51) Int. Cl. C12N 15/90; A61K 39/00; A61K 40/13; A61K 40/30; A61K 40/46; C07K 14/705; C07K 16/114; C12N 9/22; C12N 15/11; C12N 15/86

(52) U.S. Cl. CPC C12N 15/907; A61K 40/13; A61K 40/30; A61K 40/46; C07K 14/70503; C07K 16/114; C12N 9/226; C12N 15/11; C12N 15/86; A61K 2039/505; C07K 2317/565; C07K 2317/76; C07K 2317/92; C12N 2310/20; C12N 2750/14143

(57) ABSTRACT

The disclosure provides methods for promoting affinity maturation, and in particular in vivo affinity maturation, of antibodies. The disclosure also provides a system of affinity maturation of an antibody as well as compositions comprising antibodies generated from methods described herein and polynucleotides encoding such systems.

### REFERENCE TO AN ELECTRONIC SEQUENCE LISTING

The contents of the electronic sequence listing (U119770206WO00-SEQ-GIC.xml; Size: 43,104 bytes; and Date of Creation: Sep. 29, 2023) is herein incorporated by reference in its entirety.

### BACKGROUND

Antibodies can be used to treat a wide range of diseases. They are usually safe, their Fc domains confer long half-lives in the bloodstream, and they can engage a range of immune effector functions that amplify their activities. Accordingly, many approaches have been developed to improve the affinity of human antibodies (1). Nonetheless these strategies have not succeeded fully emulating the process of affinity maturation in a mammalian lymph-node germinal center. In vivo affinity maturation of B-cell receptors (BCRs) has key advantages over cell-culture or phage-based approaches. For example, in vitro selection systems require discrete selection steps, usually less than 10, often without further diversification between steps. In contrast, diversification and selection are continuous and coordinated in vivo. In vivo competition among BCRs is sensitive to small affinity differences (2, 3), whereas in vitro selection conditions are frequently less discriminating. Finally, unlike in vitro selection, in vivo selection counter-selects against BCRs that bind self antigens (4) that would otherwise limit the bioavailability of the resulting antibody. In short, the mammalian germinal center remains the best system for improving an antibody intended for human use.

Although it is straightforward to generate new antibodies in animals through vaccination and isolation of high-affinity BCRs, approaches for improving existing antibodies are more limited. In some cases, transgenic mice have been generated that express a mature or progenitor form of an HIV-1 broadly neutralizing antibody (bNAb), often to test a vaccination strategy (5-7). However, this process is cumbersome, and a new mouse must be developed for each target antibody, rendering it impractical as a common laboratory technique. Moreover, this approach cannot be extended to non-human primates, advantageous for their longevity and similarity to humans. Finally, only a single antibody, rather than, for example, antibody combinations or a library of antibody variants, can be introduced in this manner.

Recently, a number of studies reported the adoptive transfer of B-cells engineered with CRISPR/Cas9 to express HIV-1 bNAbs (8-13) or respiratory syncytial virus neutralizing antibodies (FIG. 7B) (9). A common approach for doing so introduces a single cassette expressing an exogenous light-chain variable and constant gene with a heavy-chain variable gene. This cassette is inserted in an intron immediately downstream of the native VDJ-recombined variable gene. When cells so edited are transferred to wild-type mice, they expand and generate neutralizing serum that reflects the potency of the original antibody. However, somatic hypermutation is less efficient than observed in unmodified animals (11, 14), and in vivo affinity maturation has not been reported with these systems. These limitations may result from continued expression of the endogenous murine VDJ-combined heavy-chain variable segment, introduction of exogenous promoters in the heavy-chain locus, disruption of splice patterns at this locus, somatic hypermutation of the light-chain constant region and of cassette regulatory elements, or lower overall BCR expression.

Adoptively transferred murine B cells engineered to express HIV-1 broadly neutralizing antibodies (bNAbs) proliferate, class switch, and secrete antibody in response to antigens. However, current B-cell editing strategies disrupt the antibody heavy-chain locus, resulting in inefficient somatic hypermutation without detectable affinity maturation.

Therefore, there is a need to develop effective B-cell editing methods which lead to greater proliferation of edited cells, more potent neutralizing sera, and more efficient and site-appropriate somatic hypermutation in response to antigen.

### SUMMARY OF THE INVENTION

The present disclosure provides methods for promoting affinity maturation, and in particular in vivo affinity maturation of antibodies. These methods involve CRISPR-based editing of a native coding locus of the BCR. They involve the replacement of this locus in a B cell obtained from a subject (such as a mouse) with a native locus of another organism, such as a human (or a humanized locus). In accordance with these methods, murine B cells can be edited so that their heavy and light variable chains can be directly replaced by those of a human bNAb, while preserving the organization and regulation of the B-cell receptor locus. As such, the disclosed methods may comprise the replacement of a native murine BCR locus with a native human BCR locus, including a commercially relevant human BCR locus, in a B cell that is subsequently adoptively transferred to a mouse subject for somatic hypermutation (SMH) and affinity maturation. As such, the present disclosure provides methods of editing B cell receptors, and nucleic acid molecules encoding an engineered BCR. BCRs edited by any of the methods described herein are able to affinity mature, which may facilitate development of more potent bNAb variants, and enable engineered B cells to respond adaptively to diverse or changing antigens. The present disclosure thus also provides methods of engineering B cells, and engineered B cells. Further provided are variants of human antibodies that have been affinity-matured using any of the disclosed methods, and engineered B cells expressing any of these variants. Further provided herein are therapeutic applications, such as methods of treatment involving administration to a subject in need thereof and pharmaceutical compositions, of the disclosed human antibody variants and/or engineered B cells. Also provided are systems of affinity maturation.

The disclosed methods may be applied to promote the affinity maturation of existing antibodies (such as commercial and/or FDA-approved monoclonal antibodies) or newly discovered antibodies. The disclosed methods may be applied to promote the affinity maturation of biologics derived from antibodies (such as commercial and/or FDA-approved single-chain antibody fragments or other antibody-derived drug products) or newly discovered biologics derived from antibodies.

The disclosed methods may be applied to promote mutation and affinity maturation of a human antibody, or a rodent antibody. The disclosed methods may be applied to affinity mature a library of antibodies, such as a combinatorial library of human antibodies. In some embodiments, the disclosed methods are applied to affinity mature an bNAb associated with HIV-1. The disclosed methods may be used to generate human antibody variants that have higher potency (or affinity, or avidity), bioavailability, or half-life against a target antigen than the unmutated antibody. The disclosed methods enhance the affinity or potency of antibodies while avoiding the tendency to generate antibodies that interact with self antigens or that are easily proteolyzed-which are pitfalls of existing in vitro techniques such as phage and yeast display. This disclosure provides a proof-of-concept for novel methods of in vivo affinity maturation of human BCRs and engineering of B cells (e.g., murine B cells) containing same.

Further contemplated herein are methods for adoptively transferring an engineered B cell into a subject, such as a mammalian subject, to promote affinity maturation of the human antibody under mutation (or evaluation) in the subject (in the germinal center or lymph nodes). In various embodiments, the subject is a murine subject (e.g., a mouse). In some embodiments, the mammalian subject is a rodent. In some embodiments, the mammalian subject is a wild-type mouse. In some embodiments, the mammalian subject is not a transgenic mouse.

Accordingly, the present disclosure provides improved B-cell receptor editing methods. The methods comprise contacting a B cell obtained from a mammalian subject with a homology-directed repair (HDR) template comprising a first sequence encoding heavy chain variable genes of a human antibody, a clustered regularly interspaced short palindromic repeats (CRISPR)-associated protein (Cas), and a guide RNA, wherein the B cell comprises a genomic locus encoding a B cell receptor (BCR), and wherein the guide RNA comprises a sequence having complementarity to the genomic locus; wherein the Cas protein introduces a double-strand DNA break immediately adjacent to a target site in the genomic locus, and the target site is replaced with the first sequence through HDR, thereby generating an engineered BCR; and administering the B cell comprising the engineered BCR to the subject. In various embodiments, the target site is a coding sequence in the genomic locus. As such, the disclosed methods comprise the direct replacement of one BCR-coding sequence (such as a murine BCR-coding sequence) with another (such as a human BCR-coding sequence).

The disclosure also provides methods comprising contacting a B cell obtained from a mammalian subject with comprising a first sequence encoding light chain variable genes of a human antibody, a Cas protein, and a guide RNA, wherein the B cell comprises a genomic locus encoding a BCR, and wherein the guide RNA comprises a sequence having complementarity to the genomic locus; whereby the Cas protein introduces a double-strand DNA break immediately adjacent to a target site in the genomic locus, and the target site is replaced with the first sequence through HDR, thereby generating an engineered BCR; and administering the B cell comprising the engineered BCR to the subject.

The disclosure also provides methods comprising contacting a B cell obtained from a mammalian subject with an HDRT comprising a first sequence encoding heavy chain variable genes of a human antibody and a second sequence encoding light chain variable genes of a human antibody, a Cas protein, and a guide RNA, wherein the B cell comprises a genomic locus encoding a BCR, and wherein the guide RNA comprises a sequence having complementarity to the genomic locus; wherein the Cas protein introduces a double-strand DNA break immediately adjacent to a target site in the genomic locus, and the target site is replaced with the first sequence through HDR, thereby generating an engineered BCR; and administering the B cell comprising the engineered BCR to the subject.

The present disclosure provides methods comprising contacting a mature B cell obtained from a wild-type murine subject with an HDR template (HDRT) comprising a first sequence encoding heavy chain variable genes of a human antibody and/or an HDR template comprising a second sequence encoding light chain variable genes of the human antibody, a Cas protein, and a guide RNA, wherein the B cell comprises a genomic locus encoding a BCR, and wherein the guide RNA comprises a sequence having complementarity to the genomic locus; wherein the Cas protein introduces a double-strand DNA break (DSB) immediately adjacent to at least one target site in the genomic locus; and administering the B cell comprising the engineered BCR to the subject; wherein the at least one target site is replaced with the first sequence and/or the second sequence through HDR, thereby generating an engineered BCR. In some embodiments, the guide RNA has a sequence of between 15 and 200 nucleotides that is complementary to the genomic locus. The length of the HDRT, or homology arm(s), may vary depending on the target sequence. In some embodiments, the HDRT ranges from a length of 150-250 nucleotides (nt), 250-350 nt, 300-400 nt, 400-500 nt, 300-500 nt. 350-550 nt. 500-600 nt. 500-750 nt, 600-800 nt, 700-900 nt. 750-1000 nt, or greater than 1000 nt.

The present disclosure also provides methods comprising contacting a mature B cell obtained from a mammalian subject with a first HDR template comprising a first sequence encoding a heavy chain of a human antibody and a second HDR template comprising a second sequence encoding a light chain of the human antibody, a Cas protein, and a guide RNA, wherein the B cell comprises a genomic locus encoding a BCR, and wherein the guide RNA comprises a sequence having complementarity to the genomic locus; wherein the Cas protein introduces a double-strand DNA break immediately adjacent to a first target site in the genomic locus and a second target site in the genomic locus; and administering the B cell comprising the engineered BCR to the subject; wherein the first target site is replaced with the first sequence, and the second target site is replaced with the second sequence, thereby generating an engineered BCR. In some embodiments, the first target site is a VH sequence and the light chain is a VK sequence.

In embodiments of the disclosed methods, the replacement of the target site with the first sequence does not result in integration of any exogenous genetic regulatory elements into the locus encoding the BCR. As such, the organization of regulatory elements of the BCR locus of the disclosed antibody variants is endogenous and remains preserved. In some embodiments, the replacement of the target site with the first sequence or the second sequence does not result in integration of the first sequence or the second sequence into an non-coding sequence such as an intronic sequence or other regulatory element.

In some embodiments, the Cas protein is a Cas9, Cas12a or Cas13 protein. In some embodiments, the method generates an affinity-matured antibody in the subject that is a variant of the human antibody under mutation. In some embodiments, the method results in somatic hypermutation and affinity maturation in the subject. In some embodiments, the method provides rates of somatic hypermutation of about 0.5%-5%, 0.8%-5%, 1%-5%, 1.2%-5%, 1.5%-5%, 1.8%-5%, 2%-5%, 2.5%-5%, 2.8%-5%, 3%-5%, 3.5%-5%. In some embodiments, the somatic hypermutation rate is measured by next generation sequencing. e.g., sequencing the coding region of the introduced BCR locus or loci, counting the number of amino-acid and nucleotide changes from the sequence that was introduced, and determining the percentage of changes per site, and the average percentage of changes across the entire locus or loci. In some embodiments, the affinity-matured antibody has enhanced affinity to an antigenic target relative to the human antibody.

In some embodiments, the affinity-matured human antibody has enhanced bioavailability in the subject relative to the unmutated human antibody. Bioavailability may be assessed by measuring the degree to which the affinity-matured antibody binds non-selectively to a cell membrane. In some embodiments, the bioavailability is impacted by at least pharmaceutical factors, patient related factors and routes of administration (e.g., subcutaneous or intravenous infusion). In some embodiments, bioavailability is evaluated by infusing between about 1 and about 40 mg/kg of the antibody into a human FcRn transgenic mouse (see Methods Mol Biol. 2010; 602:93-104) or macaque and monitoring the amount of that antibody present in the blood by ELISA over a roughly 30 to 90 day period. In particular embodiments, the amount of antibody is monitored over a 30-40, 40-50, 50-60, 60-70, 70-80, or 80-90 day period. In some embodiments, the disclosed antibody variants exhibit low affinity to self antigens. Accordingly, In some embodiments, the disclosed antibody variants exhibit low polyreactivity.

In some embodiments, the affinity-matured human antibody has an enhanced half-life in the subject relative to the unmutated antibody. Half-life may be assessed in vitro, such as in a cell culture assay. Half-life may be assessed through an in vivo assay, such as serum concentration over time in a subject. In some embodiments, the affinity-matured antibody has a half-life that is substantially equivalent to that of the unmutated antibody.

In some embodiments, the heavy chain variable genes of the human antibody comprise recombined germline VDJ segments. In some embodiments, the light chain variable genes of the human antibody comprise recombined germline VDJ segments. In some embodiments, the heavy chain of the human antibody comprises a heavy-chain complementarity-determining region 3 (HC-CDR3). In some embodiments, the antigenic target is a soluble protein antigen, a transmembrane protein antigen, or a viral antigen. In some embodiments, the antigenic target is an HIV antigen. In some embodiments, the antibody is an FDA-approved therapeutic antibody. In some embodiments, the antibody is the HIV-1 bNab 10-1074, or a variant thereof. In some embodiments, the heavy chain is a VH sequence, and the light chain is a VK sequence of 10-1074.

In some embodiments, the HDRT is comprised within a double-stranded DNA (dsDNA) vector. In some embodiments, the HDRT is comprised within an adeno-associated viral (AAV) vector. In some embodiments, the AAV vector is encapsidated in an AAV6 or AAV-DJ capsid.

The present disclosure provides an affinity-matured antibody variant generated using the methods described herein. Further provided herein are four polyreactivity-enhanced variants of the 10-1074 antibody, wherein these variants contain mutations in the IgH region. These variants exhibited a two-fold higher potency against a global isolate panel, in neutralizing plasma against the antigen. These variants were generated in accordance with the disclosed methods. Any of the disclosed antibody variants may be used to treat a subject suffering from or diagnosed with a disease, disorder or condition, such as any infectious disease. In some embodiments, the disclosed antibody variants may be used to treat HIV. The disclosure also provides an engineered mature B cells generated using the method described herein. In some embodiments, engineered primary B cells containing any of the described 10-1074 variants are provided. In some embodiments, the disclosure provides methods of administering the antibody variant generated from methods described herein to a subject. In some embodiments, the subject is a human.

In some embodiments, the disclosure also provides a B cell comprising the antibody variant generated by methods described herein. In some embodiments, the disclosure provides a population of B cells generated by any of the methods described herein.

The present disclosure provides a system of affinity maturation of an antibody, comprising a B cell obtained from a mammalian subject with a homology-directed repair (HDR) template comprising a first sequence encoding a heavy chain of an antibody and/or an HDR template comprising a second sequence encoding a light chain, wherein the B cell comprises a genomic locus encoding a BCR, a Cas protein, a guide RNA, wherein the guide RNA comprises a sequence having complementarity to the genomic locus, and an injection mechanism for administering the B cell comprising the engineered BCR to the subject. In some embodiments, the injection mechanism is adapted for subcutaneous or intraperitoneal injection.

The disclosure also provides a nucleic acid molecule encoding an engineered murine B cell receptor (BCR) comprising a VH domain derived from a human antibody, and a VK domain derived from a human antibody, wherein the human antibody recognizes an HIV antigen, and wherein the nucleic acid molecule comprises endogenous murine BCR regulatory elements. In some embodiments, the human antibody is 10-1074, or a variant thereof. In some embodiments, the human antibody is VRC26.25, or a variant thereof. The 10-1074 and VRC26.25 antibodies bind antigens in the Env protein of HIV.

In one aspect, the disclosure provides a method comprising contacting a B cell obtained from a mammalian subject with a homology-directed repair (HDR) template comprising a sequence encoding heavy chain variable region CDR3 of a human antibody, a Cas protein, and a guide RNA, wherein the B cell comprises a genomic locus encoding a B cell receptor (BCR), and wherein the guide RNA comprises a sequence having complementarity to the genomic locus; whereby the Cas protein introduces a double-strand DNA break immediately adjacent to a target site in the genomic locus, and the target site is replaced with the first sequence through HDR, thereby generating an engineered BCR; and administering the B cell comprising the engineered BCR to the subject.

In some embodiments, the replacement of the target site with the sequence does not result in integration of any exogenous genetic regulatory elements into the locus encoding the BCR.

In some embodiments, the mammalian subject is a rodent. In some embodiments, the mammalian subject is a wild-type mouse. In some embodiments, the mammalian subject is not a transgenic mouse.

In some embodiments, the Cas protein is Cas9, Cas12a or Cas 13. In some embodiments, the guide RNA comprises a sequence of between 15 and 200 nucleotides that is complementary to the genomic locus.

In some embodiments, the method generates an affinity-matured antibody in the subject that comprises a variant of the heavy chain variable region CDR3 of the human antibody. In some embodiments, the method results in somatic hypermutation and affinity maturation in the subject. In some embodiments, the affinity-matured antibody has enhanced affinity to the antigenic target relative to the human antibody. In some embodiments, the affinity-matured antibody has enhanced bioavailability in the subject relative to the human antibody.

In some embodiments, the antigenic target of the human antibody is a soluble protein antigen, a transmembrane protein antigen, or a viral antigen, optionally an HIV antigen. In some embodiments, the antigen is an HIV-1 Env trimer. In some embodiments, the antigen comprises the V2-glycan apex epitope. In some embodiments, the human antibody is VRC26.25 or a variant thereof.

In some embodiments, the HDR template (HDRT) is comprised within a double-stranded DNA (dsDNA) vector. In some embodiments, the HDR template (HDRT) is comprised within an adeno-associated viral (AAV) vector. In some embodiments, the AAV vector is encapsidated in an AAV6 or AAV-DJ capsid.

In one aspect, the disclosure provides a method of evaluating the immunogenicity of an antigen, comprising: I) contacting a B cell with a homology-directed repair (HDR) template comprising a sequence encoding heavy chain variable region CDR3 of a human antibody that specifically binds the antigen, a Cas protein, and a guide RNA, wherein the B cell comprises a genomic locus encoding a B cell receptor (BCR), and wherein the guide RNA comprises a sequence having complementarity to the genomic locus; whereby the Cas protein introduces a double-strand DNA break immediately adjacent to a target site in the genomic locus, and the target site is replaced with the first sequence through HDR, thereby generating an engineered BCR; II) administering the B cell comprising the engineered BCR to a mammalian subject; III) administering the antigen to the mammalian subject, and IV) measuring proliferation, class-switching, and/or affinity maturation of the engineered B cell or engineered BCR.

In some embodiments, the replacement of the target site with the sequence does not result in integration of any exogenous genetic regulatory elements into the locus encoding the BCR.

In some embodiments, the mammalian subject is a rodent. In some embodiments, the mammalian subject is a wild-type mouse. In some embodiments, the mammalian subject is not a transgenic mouse.

In some embodiments, the Cas protein is Cas9, Cas12a or Cas 13. In some embodiments, the guide RNA comprises a sequence of between 15 and 200 nucleotides that is complementary to the genomic locus.

In some embodiments, the method generates an affinity-matured antibody in the subject that comprises a variant of the heavy chain variable region CDR3 of the human antibody. In some embodiments, the method results in somatic hypermutation and affinity maturation in the subject. In some embodiments, the affinity-matured antibody has enhanced affinity to the antigenic target relative to the human antibody. In some embodiments, the affinity-matured antibody has enhanced bioavailability in the subject relative to the human antibody.

In some embodiments, the antigen is a soluble protein antigen, a transmembrane protein antigen, or a viral antigen, optionally an HIV antigen. In some embodiments, the antigen is an HIV-1 Env trimer. In some embodiments, the antigen comprises the V2-glycan apex epitope. In some embodiments, the human antibody is VRC26.25 or a variant thereof.

In some embodiments, the HDR template (HDRT) is comprised within a double-stranded DNA (dsDNA) vector. In some embodiments, the HDR template (HDRT) is comprised within an adeno-associated viral (AAV) vector. In some embodiments, the AAV vector is encapsidated in an AAV6 or AAV-DJ capsid.

### BRIEF DESCRIPTION OF DRAWINGS

FIGS. 1A-1G show a comparison of an exemplary disclosed native-loci editing approach for engineering mouse B-cell receptors with an intron editing strategy. FIG. 1A is a representation of a native-loci editing approach in which homology-directed repair templates (HDRTs) are used to directly overwrite mouse heavy and kappa chains with exogenous heavy and light variable chains. Murine B cells were electroporated with a Mb2Cas12a ribonucleoprotein (RNP) complex targeting the murine JH4 (heavy chain, top) or Jκ5 (light chain, bottom) segments (scissors). HDRTs were delivered as double-stranded DNA (dsDNA) or via a recombinant adeno-associated virus (AAV). The heavy-chain HDRT (top) encodes the recombined heavy-chain variable gene of a human antibody bounded by homology arms complementary to sequence encoding the promoter and leader peptide of the VH1-85 or VH1-64 variable segments (5′ homology arm) and to the intronic region downstream of JH4 (3′ homology arm). The light-chain HDRT (bottom) similarly encodes a recombined kappa-chain variable region of the same antibody, with homology arms complementary to the Vκ1-135 promoter and leader-peptide sequence (5′ homology arm) and the intronic region downstream of Jκ5 (3′ arm). FIG. 1B shows that primary murine B cells were edited as shown in FIG. 1A and analyzed by flow cytometry using the same soluble HIV-1 envelope glycoprotein trimer (CRF250-SOSIP) conjugated to APC (horizontal axis) or PE (vertical axis). The bNAb introduced (10-1074 or the VRC26.25 variant VRC26.25-y (20)), the method of HDRT delivery (dsDNA or AAV), and the 5′ homology arms of the heavy (VH1-64 or VH1-85) and kappa (Vκ1-135) are indicated above each plot. Control cells were electroporated with the same RNP complex, without HDRT (no HDRT). Numbers indicate the percentage of cells in the SOSIP-positive gate, demarked in bold lines. FIG. 1C provides a summary of results from four to six independent experiments similar to those shown in FIG. 1B, using AAV vectors to template homology-directed repair. Error bars represent the standard error of the mean (SEM). It was shown that the heavy chain of VRC26.25-y alone bound CRF250-SOSIP. In contrast, both 10-1074 chains are necessary to bind this SOSIP. FIG. 1D is a representation of an alternative editing approach in which a bicistronic cassette is introduced in the intron between JH4 and gene segments encoding the constant domain. The cassette shown is identical to that described in Hartweger et al. (8) and similar to that used in References 9-12. This cassette initiates with a splice acceptor site and an SV40 polyadenylation signal designed terminate the murine heavy-chain VDJ message, the IgHV4-9 promoter which drives expression of an exogenous VJ-recombined human light-chain, a murine Cκ gene, a furin cleavage site, a 3-residue linker, a P2A self-cleaving sequence, an exogenous VDJ-recombined human heavy-chain gene, and a splice donor. This cassette is bounded by homology arms complementary to the intronic region. In parallel, the endogenous murine kappa is eliminated by NHEJ-mediated repair of a double-strand break in Cκ, as in Hartweger et al. (8). FIG. 1E shows an experiment similar to that shown in FIG. 1B except that the intron-targeting approach represented in FIG. 1D was used. FIG. 1F shows a summary of results from two to four independent experiments similar to those shown in FIG. 1E. Error bars represent SEM. FIG. 1G shows IgM expression three days post-electroporation with RNP and transduction with AAV-HDRT, primary murine B cells were analyzed by flow cytometry with an anti-IgM antibody and APClabeled CRF250-SOSIP. Fully IgM-negative cells, likely resulting from imprecise NHEJ-mediates repair, were excluded, and the remaining cells were gated into CRF250 SOSIP-positive (light gray) and -negative (dark gray) populations and plotted according to their IgM geometric mean fluorescence intensity (gMFI). SOSIP-reactive cells engineered in their native loci expressed higher IgM levels than intron edited cells. Dotted line reflects the mean IgM levels observed in unedited murine cells. Error bars indicate SEM from two (VRC26.25-y) or three (10-1074) independent experiments. Significant results are indicated (*p<0.05; **p<0.01; unpaired two tailed t-test).

FIGS. 2A-2G show that B-cells engineered with native-loci editing generate potent HIV-1 neutralizing plasma in vivo. FIG. 2A shows the schedule of immunization and blood collection used to generate plasma and cells analyzed in FIGS. 2B-2G. Murine B cells were obtained from C57BL/6 mice marked with the CD45.1 variant, engineered with either native-loci or intron editing approaches, and adoptively transferred to CD45.2-positive recipient mice. These latter mice were immunized at day 1 and weeks 3, 6, 9 and 12 after transfer. Blood was collected days 14, 28, 46, 67, 88, and day 109. FIG. 2B shows neutralization studies against the CRF250 HIV-1 isolate, homologous to the CRF250-SOSIP antigen. Panels characterize plasma after each of five vaccinations from mice engrafted with the same numbers of SOSIP-reactive native-loci- and intron-edited B cells. Each panel compares plasma from three native-loci-edited (dark blue solid line), from three intron-edited (light blue solid line) mice, and from naïve mice in which 20 μg/ml of 10-1074 with a murine (light purple dotted line) or human (black dotted line) constant domain. Control (gray solid line with circles) indicates identically vaccinated mice without adoptive transfer. Error bars indicate SEM of duplicates. FIG. 2C shows a series of neutralization studies similar to those in FIG. 2B except native-loci introduced VRC26.25-y (orange solid line) was compared to its intron edited counterpart (yellow solid line). Black and green dotted lines indicate plasma obtained from wild-type mice in which 2 μg/ml of wild-type VRC26.25 or the VRC26.25-y variant, respectively, is characterized. Gray solid line with squares indicates identically vaccinated control mice without adoptive transfer. Error bars indicate SEM of duplicates. FIG. 2D shows a summary of 50% inhibitory dilution (ID₅₀) values from FIGS. 2B-2C. Comparisons between native-loci- and intron-editing are significant after the first immunization for 10-1074 (p<0.0001; unpaired two-tailed t test) and VRC26.25-y (p<0.05) and after the third immunization for VRC26.25-y (p<0.05). Error bars represent SEM for each group of three mice. FIG. 2E shows a neutralization study similar to that in FIG. 2C except that neutralization of heterologous isolates CNE55 (clade AE), WITO (clade B), and BG505 (clade A1) was measured. Error bars indicate SEM of duplicates. FIG. 2F shows a summary of ID₅₀ values from FIG. 2E. Comparisons between native-loci and intron editing are significant for CNE55 (p<0.05: unpaired two-tailed t test). Error bars represent SEM for each group of three mice. FIG. 2G shows that B-cells from spleen, lymph nodes, and bone marrow of 10-1074 and VRC26.25-y mice were analyzed 7 days after the final immunization. Antigen-reactive donor cells memory B cells were determined as the ratio of CRF250-SOSIP+, CD45.1+ cells to total memory B cells (CD19+, CD38+, GL7−). A similar ratio was determined form germinal center (GC) B cells (CD19+, CD38−, GL7+) and plasma cells isolated from spleen and lymph nodes (CD138+, CD19+/−) and from bone marrow (CD138hi) (35, 36). Error bars indicate SEM from 3 mice in each group. Significant results are indicated (*p<0.05; **p<0.01, unpaired two-tailed t test).

FIGS. 3A-3C show how VRC26.25-y and 10-1074-expressing B cells can be combined to provide broader protection. FIG. 3A shows that CD45.2 mice were engrafted with B cells edited at their native loci to express 10-1074 (blue). VRC26.25-y (red), or both (green) vaccinated, and blood was collected as shown in FIG. 2A. Neutralization activity (mean ID₅₀ for the two mice of each group) was measured after each vaccination against the CRF250 pseudovirus. No neutralization was observed with unengrafted controls vaccinated in the same manner. Error bars indicate SEM. FIG. 3B shows the serum neutralization activity against heterologous isolates resistant to either VRC26.25-y (JR-FL) or 10-1074 (X1632, CNE55) after the 5th and 6th immunizations. Error bars indicate SEM of duplicates. FIG. 3C shows a summary of ID₅₀ values obtained from FIG. 3B. Mice transferred with a mixture of 10-1074- and VRC26.25-y-engineered B cells neutralized all three tested isolates.

FIGS. 4A-4C show how native loci editing enables more efficient somatic hypermutation. FIG. 4A shows that donor B cells from spleen and lymph nodes in VRC26.25-y and 10-1074 recipient mice characterized in FIGS. 2A-2G were sequenced 7 days after their final vaccination. This figure also shows the average frequency of amino acid substitution in a sliding window of 10-residues centered on the indicated VRC26.25-y residue, observed in three native-loci-edited cells (top, red, magenta, orange) and two intron-edited cells (bottom, blue and black). Boxes indicate heavy- and light-chain CDRs. Note the higher frequency of SHM, especially in the CDR regions, in native loci-edited mice. Corresponding 10-1074 results are presented in FIG. 12A. FIG. 4B shows the average frequency of heavy- and light-chain mutations in native-loci-(left) and intron-edited (right) forms of VRC26.25-y and 10-1074. FIG. 4C shows the average frequency of mutations in the CDR and FR regions is indicated. (*p<0.05; **p<0.01; ****p<0001, unpaired two-tailed t test). Results for the native-loci forms are shown at the left and results for the intron-edited forms are shown at the right for each of the CDR and FR regions on the x-axis. Error bars indicate SEM.

FIGS. 5A-5C show affinity maturation of 10-1074 in mice. FIG. 5A shows the frequency of heavy- and light-chain amino-acid mutations averaged from three mice engrafted with native-loci edited 10-1074 B cells. White arrowheads indicate positions where the most frequent mutation emerged in more than one mouse, the criteria for further characterization. Two arrowheads indicate that two different substitutions were present at the same position in more than one mouse, and both were characterized. Results for individual mice are provided in FIGS. 12B-12E. FIG. 5B shows the neutralization (IC₅₀) values against the CRF250 or BG505-T332N isolates of 10-1074 variants, each mutated at a single heavy- or light-chain position indicated in FIG. 5A. Red dotted line indicates the IC₅₀ of wild-type 10-1074 and black solid line indicates the geometric mean IC₅₀ of all variants. Light blue dots indicate mutations resulting in the lowest average IC₅₀ for both isolates. These were further characterized in FIGS. 6A-6E. More heavy-chain mutations improved potency. FIG. 5C shows that the heavy- and light-chain mutations characterized in FIG. 5B were divided into those that improved neutralization for both isolates, those that improved it for only one indicated isolate, and those that did not improve neutralization for either isolate (moving clockwise). The number of mutations characterized is indicated within each figure.

FIGS. 6A-6E show that 10-1074 variants were more potent than 10-1074 against a global-isolate panel. FIG. 6A is a table naming and describing four variants developed from data shown in FIG. 5B and FIG. 13A. Each variant includes the V100dM and S100fA mutations, with other mutations shown to enhance neutralization of BG505-T332N and CRF250 pseudoviruses. FIG. 6B shows that the polyreactivity of wild-type 10-1074 and the indicated 10-1074 variants were compared to polyreactive antibodies PGT128 and NIH45-46W by immunofluorescence assays (IFA), using Hep-2 cells and 100 μg/ml of each antibody. Error bars indicate SEM of three independent measurements. All variants retained or reduced the polyreactivity of wild-type 10-1074, and all were significantly less polyreactive than PGT128 (p<0.05) or NIH45-46W (p<0.001; unpaired two-tailed t test). Differences among 10-1074 and its variants were not significant. FIG. 6C shows the neutralization of wild-type 10-1074 compared to 10-1074-y3, the most potent variant described here, against all eight 1-1074-sensitive viruses in the global panel. The commonly studied BG505-T332N and YU2 isolates are also included, as is one of four 10-1074-resistant virus (CNE8) from the panel. Errors bars represent SEM of triplicates. A summary of IC₅₀ (FIG. 6D) and IC₈₀ (FIG. 6E) values for wild-type 10-1074 and each 10-1074 variant is shown. Resistant isolates were assigned a value of 50 μg/ml. Each point represents a mean of two independent sets of triplicates. Geometric mean values are indicated by a horizontal bar. Geometric mean values for 10-1074-sensitive isolates, for all isolates, and the breadth of each variant are indicated beneath the figure. Significance between wild-type 10-1074 and each variant was determined by a Wilcoxon matched signed-rank test (** p<0.01).

FIG. 7A shows schematics representing an exemplary native locus editing approach of this disclosure for the heavy chain and light chain of an antibody to be affinity matured. Scissors indicate a double-strand break introduced by a Cas9 CRISPR effector protein. Also shown are a series of schematics representing existing mature B-cell editing approaches. The human or mouse heavy- and kappa light-chain loci are represented, with HDRT shown underneath each chromosomal target. Scissors indicate a double-strand break introduced by a Cas9 CRISPR effector protein, except as described herein and in Ou et al., which used the CRISPR protein Mb2Cas12a. References are indicated beneath each panel (8-11, 13, 15, 41, 42).

FIGS. 8A-8F show how exogenous human heavy- and light-chain variable segments were introduced into their respective native loci in a human B cell line. FIG. 8A ia a representation of the native-loci editing approach targeting the human heavy chain locus. Jeko-1 cells were electroporated with a RNP complex targeting the 3′ region of JH6. HDRTs were delivered as double-stranded DNA (dsDNA) or via AAV6, as indicated. The HDRT encodes the recombined heavy-chain variable gene bounded by homology arms complementary to the sequence encoding the promoter of VH7-81 (5′ homology arm) and the intronic region downstream of JH6 (3′ homology arm). FIG. 8B shows a similar representation of a native-loci editing approach for overwriting the human kappa chain variable region. The light-chain HDRT similarly encodes the recombined kappa-chain variable region with homology arms complementary to the Vκ2-40 promoter sequences (5′ homology arm), and the intronic region downstream of Jκ5 (3′ arm). FIG. 8C shows Jeko-1 cells were edited as shown in FIG. 8A (IgH) or FIGS. 8A-8B (IgH+IgK) and analyzed by flow cytometry using the same soluble HIV-1 envelope glycoprotein trimer (CRF250-SOSIP) conjugated with different fluorophores. Note the markedly greater editing efficiency when HDRT is provided as an AAV vector. FIG. 8D shows different gRNA-targeting approaches were used with the same HDRT to overwrite the native Jeko-1 IgH variable-chain gene. RNP complexes were loaded with gRNA targeting the JH6 segment (JH6), the V7-81 promoter and the JH6 segment (VH7+JH6), or the same promoter and the intronic region downstream of JH6 (VH7+intron), as shown above. Jeko-1 cells were electroporated with the RNP and the indicated HDRT, and then analyzed by flow cytometry (bottom). Cells were stained with fluorescently labeled CRF250-SOSIP (vertical axis) and with an anti-IgM antibody (horizontal axis). The gate indicates the percent of cells expressing the VRC26.25 heavy chain. FIG. 8E shows a comparison between an exemplary native-loci strategy (IgH:native) shown in FIG. 8A and a JH6 replacement approach (IgH:JH6) in which a heavy-chain variable region together with a promoter is edited into the JH6 region. Edited cells were analyzed as in FIG. 8C. FIG. 8F shows a similar comparison of light-chain editing techniques in which the heavy-chain of 10-1074 was introduced with native-editing (FIG. 8A), and the light chain was introduced using native editing, or two distinct light chain cassettes precede by the Vκ3-20 promoter, with or without a polyA tail designed to terminate the endogenous kappa variable gene. These results indicate that the use of the native editing strategy for the heavy and light chains provides the highest editing efficiency.

FIGS. 9A-9B show optimized HDRTs and gRNAs in primary murine B cells. FIG. 9A shows part of the murine heavy-chain locus used to indicate the locations of V1-85, -82, -64, -55, and -53 segments. HDRTs with homology arms complementary to the indicated region in the promoter of each variable gene, and to the intronic region 3′ of JH4 were compared, along with an HDRT with 5′ homology arms complementary to the consensus of V1-family genes in the same region. Mouse primary B cells were electroporated using the same JH4-targeting RNP and HDRTs encoding the VRC26.25-y heavy chain with 5′ homology arms complementary to the indicated VH1 gene. Targeting the VH1-85 and VH1-64 genes resulted in most efficient editing. FIG. 9B shows how the multiple gRNAs cutting in the vicinity of JH4 or Jκ5 were characterized. Cut sites are indicated by arrows. Right panel indicates editing efficiency as determined by flow cytometry with CRF250-SOSIP antigens. RNP complexes were loaded with the indicated gRNAs and HDRTs targeting either VH1-64 alone (top, VRC26.25-y heavy chain) or VH1-85 plus Vκ1-135 (bottom panel; 10-1074 heavy and light chains). Red text indicates gRNAs used in figures of the main text to edit heavy (gRNA-A) and light (gRNA-2) loci.

FIGS. 10A-10C show immunogens, immunization schedules, and plasma neutralization responses in mice evaluated in the experiments of the Examples. FIG. 10A shows the immunogen and immunization schedule for 10-1074 mice (top) or VRC26.25-y mice (bottom) engrafted with native-loci- and intron-edited B cells in FIGS. 2A-2G. 10-1074 mice were immunized with the 13-0160-mer fused to the gp120 protein of BG505 (22). VRC26.25-y mice were immunized with a previously described SOSIP.v7 based on the CRF250 isolate (CRF250-SOSIP) (16, 17). Antigens were produced in GnTI-negative (GnTI−) cells or Exp293 cells, as indicated. Antigens produced in GnTI-negative cells lack complex glycans. FIG. 10B summarizes neutralization results in the bottom panel (ID₅₀s against the CRF250 isolate) when an alternative vaccination schedule, shown at top, is used. Intron-targeting (light blue) and native loci editing (dark blue) are compared after each of five immunizations. Error bars indicate SEM from 3 mice in each group. FIG. 10C shows similar results to FIG. 10B were observed when antigens for the first two vaccinations were produced in GnT1-cells. Error bars indicate SEM of 3 mice in each group.

FIGS. 11A-11E show the longitudinal tracking of engineered cells in recipient mice. FIGS. 11A-11C show the longitudinal analyses of blood-derived B cells following each immunization in the four groups of 10-1074 and VRC26.25-y mice with native-loci- or intron-edited cells described in FIGS. 2B-2F. FIG. 11A shows a panel representing the frequency of CD45.1+ donor cells in the CD19+ B-cell population. Dots represent values for individual mice. Lines represent averages for the indicated groups. FIG. 11B shows the frequency of CRF250 SOSIP-binding CD45.1+ donor cells. Note the percentage of donor cells that binds antigen increases after each immunization. FIG. 11C shows the frequency of bNAb-producing plasma cells as indicated by intracellular staining of permeabilized CD138+ cells with CRF250-SOSIP, is shown. FIG. 11D shows a representative gating strategy of memory B cells, GC B cells, and plasma cells used in FIG. 2G. FIG. 11E shows class-switched B-cells from spleen and lymph nodes of 10-1074 mice were analyzed. Class switched antigen-reactive donor memory B cells were determined as the percentage of antigen-binding IgG1+ cells to all antigen-binding donor memory B cells (CD19+, CD38+, GL7−, CD45.1+, CRF250-SOSIP+). Similarly, the ratio of class switched antigen-reactive donor GC B cells was determined as the percentage of antigen-reactive IgG1+ cells to all antigenreactive donor GC B cells (CD19+, CD38−, GL7+, CD45.1+, CRF250-SOSIP+). Error bars indicate SEM from two mice in each group.

FIGS. 12A-12E show SHM in native-loci-edited and intron-targeted cells in individual mice. FIG. 12A is a figure similar to FIG. 4A except that 10-1074-edited B cells were analyzed. FIG. 12B is a figure similar to FIG. 5A except that the frequency of heavy- and light-chain amino acid mutations in individual mice was analyzed. Horizontal axes indicate amino-acid positions. FIG. 12C shows the frequency of heavy- and light-chain amino acid mutations in two 10-1074 mice engrafted with intron-edited cells. FIG. 12D shows the frequency of heavy- and light-chain amino acid mutations in three VRC26.25-y mice engrafted with native loci-edited cells. FIG. 12E shows the frequency of heavy- and light-chain amino acid mutations in two VRC26.25-y mice engrafted with intron-edited cells.

FIGS. 13A-13F show the characterization of 10-1074 and VRC26.25-y variants. FIG. 13A shoes the neutralization curves of wild-type 10-1074 and 10-1074 variants with dual or triple combinations among N31K, E55D, V100dM, and S100fA, using pseudoviruses bearing the YU2 or BG505-T332N Env proteins, as indicated. Variants with both V100dM and S100fA significantly improved neutralization of both isolates. Errors bars represent SEM of triplicates.

FIG. 13B shows the structure of 10-1074 Fab bounded with BG505 SOSIP.664 (PDB 6UDJ). The three gp 120 subunits of BG505 SOSIP.664 are indicated in gray, yellow, and light green. Glycans resolved in the structure are indicated with dark green spheres. 10-1074 Fab fragments are show, with the 10-1074 heavy chain shown in salmon, and the light chain in light blue. Inset highlights the two CDRH3 residues, V100d and S100f, whose mutation improved neutralization of most isolate tested. V100d interacts with the glycan appended to gp120 N332, and with the ‘GDIR’ region (residues 324 to 327) and H330 at the C-terminus of the gp120 third variable loop. S100f interacts primarily with the N332 glycan. FIG. 13C shows representative views of antibody binding to HEp-2 cells measured by an immunofluorescence assay (IFA) with 100 μg/mL of the indicated bNAbs. The negative sample is serum without polyreactivity provided by the manufacturer. FIG. 13D shows a table naming and describing VRC26.25-y variants. Notably, I56S is a reversion to the original VRC26.25 V3-30 germline. FIG. 13E shows a summary of IC₅₀ values similar to that in FIG. 6D, except that VRC26.25 and its variants were characterized. Significant differences between wild-type VRC26.25 and each variant were determined by a Wilcoxon matched signed-rank test (*** p<0.001). FIG. 13F shows the corresponding IC₅₀ values to those shown in FIG. 6D and FIG. 13E. Colors indicate neutralization potency, with warmer colors indicating a higher potency. Fully resistant isolates (IC₅₀>50 μg/ml) are indicated in white.

FIGS. 14A-G show that mice engrafted with HCDR3-edited B cells generate neutralizing sera after immunization. FIG. 14A shows a structure of the VRC26.25 Fab bound to an Env trimer (PDB: 6VTT, 81). The VRC26.25 heavy-chain variable chain is indicated in a darker color, with the light-chain variable chain in a lighter color. The darker color indicates the V1V2 region formed by the V1 and V2 Env variable loops, where the apex epitope locates. Light grey color indicates the base region. Note that the long HCDR3 protrudes into a basic cavity formed by all three Env protomers. FIG. 14B represents the coding region of the murine antibody heavy-chain variable (VH) locus. The HCDR3 is encoded in VDJ-recombined heavy chain by the 3′ end of a VH gene, one or more D genes, and the 5′ end of the JH4 gene. The gRNA of the CRISPR Mb2Cas12a effector protein complements the conserved 3′ of JH4, while cleaving near the site of HCDR3 insertion for efficient editing. As indicated, JH4, the most 3′ of murine JH genes, contains optimally positioned non-canonical PAM sequence (GTTC), efficiently cleaved by the Mb2Cas12a ortholog. This PAM, the gRNA, and the Mb2Cas12a cut sites are indicated. At the top of the figure, the homology-directed repair template (HDRT) encoding an exogenous HCDR3 is shown, bounded by two short homology arms (60 to 72 nucleotides). The 5′ homology arm complements a VH1 consensus sequence and the 3′ arm complements a conserved region of JH4 and adjacent intronic sequences. FIG. 14C shows the results of the following: the HCDR3s of the apex bnAbs PG9, PG16, VRC26.25, VRC26-UCA, or a hemagglutinin (HA) tag were introduced into the BCRs of primary murine B cells and analyzed by flow cytometry for their ability to bind a fluorescently an anti-HA antibody or fluorescently labeled SOSIP trimer (16055-ConM-v8.1ds). This SOSIP trimer has the V1V2 region of 16055, and the base of conMv8.1ds. The horizontal axis indicates expression of surface IgM, and its loss indicates imprecise non-homologous end joining (NHEJ) after Mb2Cas12a-mediated cleavage. Number within each panel indicates the percentage of cells that bind an anti-HA antibody or SOSIP trimer. FIG. 14D summarizes the results of three independent experiments similar to those shown in panel C. Error bars indicate SEM. Statistical differences compared to negative controls were determined by two-way ANOVA (*p<0.05; ****p<0.0001). FIG. 14E shows a timeline of the B-cell engraftment and immunization protocol used in subsequent panels. B cells isolated from CD45.1-positive mice were engineered to express the VRC26.25 HCDR3 and engrafted at day 1. Mice were immunized with 16055-ConM-v8.1ds SOSIP conjugated to a mi3 60-mer scaffold on days 2, 16, and 30, and blood was harvested at day 9, 23, and 37 for ELISA and neutralization studies. Additional control mice were not engrafted with exogenous B cells but otherwise immunized identically. FIG. 14F shows the results of the following: sera pooled from seven mice, harvested after each indicated immunization (filled blue symbols—circle, square, inverted triangle), were evaluated for their ability to bind 16055-ConM-v8.1ds SOSIP by ELISA. Three unengrafted, immunized mice (open grey symbols) served as controls. Naïve sera (filled olive upright triangles) were obtained from mice without engraftment or immunization. FIG. 14G shows the ability of pooled sera from panel F to neutralize pseudoviruses bearing the Envs of indicated HIV-1 isolates (CRF250, 16055). Naïve sera mixed with wild-type VRC26.25 antibody to 2 μg/ml is used as a positive control (open diamonds). Additional data is provided in FIG. 21.

FIGS. 15A-I show that HCDR3-edited B cells migrate to germinal centers, class switch, and hypermutate. FIG. 15A shows a timeline of studies used in this figure. Host mice distinguished by a CD45.2 marker were adoptively transferred at day 1 with VRC26.25 HCDR3-edited B cells derived from a CD45.1 donor. Six engrafted mice were immunized with SOSIP (BG505-ConM-v8.1ds) antigens conjugated to a mi3-60mer scaffold at days 2, 16, and 30. Spleens and lymph nodes were harvested from two mice at the indicated time points (red asterisk) and SOSIP-bound donor B cells were sorted for next-generation sequencing. FIG. 15B shows the results of the following: B cells isolated from spleen and lymph nodes after each indicated immunization, or from engrafted mice without immunization, were analyzed by flow cytometry. A representative flow figure is shown for each time point. Germinal center (GC) B-cells identified as GL7+/CD38− were gated and quantified (top panels). These gated cells were then analyzed for the frequency of CD45.1+, indicating they derived from donor mice, and for SOSIP binding. Numbers in bottom panels indicate percentages of SOSIP(+) and SOSIP(−) donors B cells found in GC, respectively. FIG. 15C shows the mean percentage of GC cells from two mice harvested after each indicated immunization. Each dot represents results from a single mouse. FIG. 15D shows the mean percentage of CD45.1 donor cells gated from GC, with SOSIP(+) (crosshatches) and SOSIP(−) cells (solid) indicated. FIG. 15E shows the results of the following: VH1 gene segments of donor CD45.1 cells isolated from mice described in panels A-D were analyzed by next-generation sequencing (NGS). SOSIP-binding donor B cells [(CD45.1+/SOSIP(+)] were isolated from two mice at each time point and pooled for sequencing. The diversity and frequency of VH1 gene segments of successfully edited donor cells is represented with a heat map as percentage of total clones for the indicated time points. ‘Input’ indicates CD45.1+/SOSIP(+) cells after editing but before engraftment. Note the persistence of repertoire diversity but with enrichment for specific variable chains. FIG. 15F shows the distribution of isotypes of CD45.1+ donor cells with (insert +) or without (insert −) the VRC26.25-HCDR3. Cells were sorted before engraftment (input) or after each of the indicated immunizations. Note that a higher proportion of cells underwent class-switch recombination in the cells bearing the VRC26.25-HCDR3 insert. FIG. 15G shows the percentage of inserted HCDR3 with amino acid changes for IgM and class-switched (IgG1/2A/2B) BCR. FIG. 15H shows the frequency of IgH (position 21-105) amino acid mutations in clones expressing the inserted HCDR3 for each immunization. Grey boxes indicate HCDR1 and HCDR2 regions. FIG. 15I shows the frequency of VH1-64 (position 21-105) amino acid mutations in clones expressing the inserted HCDR3 after the third immunization. Each amino acid represented with the indicated color. The germline sequence of VH1-64 is shown below the figure. Additional data is provided in FIG. 22.

FIGS. 16A-C show the affinity maturation of HCDR3-edited B-cell receptors. FIG. 16A shows the frequencies of amino acid mutations within the HCDR3 region present in edited clones were analyzed after the indicated immunization. The most frequent 9 mutations observed in more than one mouse group represented here, are marked with triangles, with colors indicating the mutated amino acid. These mutations are characterized in subsequent panels. Note that the first and last residues shown derive from murine VH1-family and JH4 segments, respectively, rather than the VRC26.25 HCDR3. FIG. 16B shows the results of the following: Top panel: combinations of HCDR3 mutations identified in FIG. 22C as high affinity binders to ConM-SOSIP (v8.1ds) immunogens bearing either the CRF250 or BG505 V1V2 regions were introduced into full-length, unmodified VRC26.25. Bottom panels: these VRC26.25 variants were characterized for their ability to neutralize the indicated pseudoviruses, including two VRC26.25-resistant isolates (WITO and TRO11). FIG. 16C shows the results of the following: the same VRC26.25 variants were used to measure neutralization efficiency of the indicated global panel of HIV-1 isolates. IC₅₀ values are represented in colored circles, with geometric mean values indicated by a line. Significant differences from wild-type VRC26.25 were determined by paired t-test (*** p<0.001). Additional data is provided in FIG. 23.

FIGS. 17A-E show that HCDR3-edited B cells facilitate evaluation of SOSIP antigens. FIG. 17A shows an example of flow cytometry analysis comparing the binding efficiencies of the indicated SOSIP variants to a population of murine cells edited to express the VRC26.25 HCDR3. Note that all SOSIP proteins included the VIV2 region of the isolate 16055, which replaces the native V1V2 region of the BG505 or ConM SOSIP proteins. Base version indicates sets of previously described mutations designed to stabilize the indicated SOSIP protein (see FIG. 23A). SOSIP binding and IgM expression are indicated on the vertical and horizontal axes, respectively, and the percentage of SOSIP-binding cells is indicated within the gate. FIG. 17B shows a summary of results from experiments similar to those shown in panel A, with each dot representing an independent experiment. Percentage and MFI for each base version on the x-axis are shown on the left and right, respectively. FIG. 17C shows the schedule of immunization with the indicated mi3-multimerized SOSIP proteins of groups of seven mice adoptively transferred with VRC26.25 HCDR3-edited B cells. The neutralizing response against the indicated pseudoviruses was measured using serum from each mouse obtained seven days after the second immunization. Lines indicate median 50% inhibitory dilution (ID₅₀)), and grey dots (towards the bottom of the y-axis) indicate results from each of three unengrafted mice immunized in the same manner. FIG. 17D shows the results of the following: the ability of sera from these mice to bind to 16055-ConM-v8.1ds SOSIP was determined by ELISA. The left bar in each pair indicates pooled sera from 7 immunized mice engrafted with HCDR3-edited B cells, and the right bar in each pair) indicated pooled sera from 3 mice immunized without engraftment of edited B cells. Figure shows one of two independent experiments, each with three technical replicates, with similar results. FIG. 17E shows the results of the following: competition ELISA assays were performed to evaluate the specificity of apex antibody responses. Specifically, the indicated sera analyzed in panel C was used to prevent binding of VRC26.25 or the germline form of VRC26.25 to 16055-ConM SOSIP. The percentage of competition was calculated based on the binding of germline or mature VRC26.25 in the presence of naive mouse serum. The grey in the second and fourth panels indicates sera from immunized mice without engraftment with edited B cells. Each point represents serum from one mouse. Significant differences in panels B-E were determined by one-way ANOVA (ns, not significant; *p<0.05; **p<0.01; ***p<0.001; ****p<0.0001). Additional data is provided in FIG. 24 and FIG. 25.

FIGS. 18A-C show a comparison of the V1V2 regions from different isolates. Experiments are similar to those shown in FIG. 17A-C, except that ConM 8.1 SOSIP variants were modified with V1/V2 regions from the indicated HIV-1 isolates. Significant differences in panels B and C were determined by one-way ANOVA (ns, not significant; *p<0.05; **p<0.01; ***p<0.001; ****p<0.0001). Percentage and MFI for each base version on the x-axis are shown on the left and right, respectively.

FIGS. 19A-E show that SOSIP-TM proteins expressed from mRNA vaccines raise more potent neutralizing responses than multimeric protein antigens. FIG. 19A shows an experiment similar to those performed in FIG. 17C except that the indicated amounts of mRNA-LNP expressing the indicated SOSIP-TM proteins were compared to the soluble forms of the same SOSIP molecules conjugated to the mi3 60-mer scaffold, with prime and boost separated by two or three weeks, as indicated. Sera harvested one week after the boost immunization were characterized. Grey (towards the bottom of the y-axis) indicates sera from unengrafted mice immunized in the same manner. Statistical difference was determined by one-way ANOVA.

FIG. 19B shows the frequency of HCDR3 amino acid mutation of donor B cells from immunized mice. Mice were immunized three times with 5 μg mRNA expressing the 16055-ConM-v8.1ds, with each immunization separated by three weeks. The first and last residues shown derive from murine VH1-family and JH4 segments, respectively, rather than the VRC26.25 HCDR3. FIG. 19C shows the following: mRNA-LNP expressing the indicated SOSIP-TM variant were used to transfect 293T cells. Cells were then analyzed by flow cytometry for their ability to bind 2G12, an antibody recognizing conserved gp120 glycans and used here to monitor the overall cell-surface expression, and by VRC26.25. FIG. 19D shows a summary of mean fluorescent intensities for experiments similar those shown in panel C is presented at the left, and the ratio of VRC26.25 normalized to 2G12 binding is shown at the right. Panels C and D are representative of three independent experiments with two technical replicates for each sample. FIG. 19E shows an experiment similar to those shown in FIG. 17C and FIG. 19A except that sera from mice vaccinated with mRNA-LNP expressing the indicted SOSIP-TM proteins were compared using the indicated pseudoviruses. Significant differences were determined by one-way ANOVA (ns, not significant; *p<0.05; **p<0.01; ***p<0.001; ****p<0.0001). Additional data is provided in FIG. 26.

FIGS. 20A-D show that mRNA-LNP elicit neutralizing sera in mice engrafted with B-cells engineered to express the HCDR3s of PG9, PG16, or the VRC26-UCA. FIG. 20A shows the following: the indicated numbers of mice per group were engrafted with B cells expressing the HCDR3 of the apex bnAbs PG9, PG16, CH01, and the VRC26-UCA. Serum was harvested one week after each of three immunizations with mRNA-LNP encoding the 16055-ConM-v8.1 SOSIP-TM (PG9, PG16, CH01) or CRF250-ConM-v8.1ds (VRC26-UCA). ID₅₀ values were determined using the CRF250 or 16055 pseudoviruses with sera collected from each mouse after the indicated immunization. Grey indicates sera from unengrafted mice immunized in the same manner. FIG. 20B shows a phylogenetic tree that was constructed from the most frequently observed clones in mice engrafted with B cells expressing the VRC26-UCA HCDR3. The mature VRC26.25 HCDR3 is shown for reference. The UCA lacks an internal disulfide bond present in the mature HCDR3 (arrows). The same mutations that also emerged in the human VCR26-lineage were highlighted with different shades of blue, indicating the number of weeks post HIV-1 infection when these mutations first identified. Note the high hypermutation frequency occurred in the vicinity of this missing disulfide bond. The five most frequently observed HCDR3 are indicated with a unique designation (U1-U5). FIG. 20C shows the results of the following: the HCDR3 of mature VRC26.25 was replaced by that of VRC26-UCA or the indicated UCA variants and characterized for their ability to neutralize CRF250 pseudoviruses.

FIG. 20D shows a summary of IC₅₀ values calculated from three replicates determined as in panel C is shown. See additional analysis in FIG. 27.

FIGS. 21A-C show the optimization of HCDR3 editing. The studies are related to those shown in FIG. 14. FIG. 21A shows the results of the following: the HA tag-encoding homology-directed repair template (HDRT) used in FIG. 14C was modified with one to three phosphorothioate (PS) bonds at the 5′ or 3′ end or both, as indicated. These HDRT were used to introduce the HA tag into the HCDR3 region of primary murine B cells. Cells were then analyzed by flow cytometry with an anti-HA (vertical axis) and anti-mouse IgM antibody (horizontal axis). The percentage of HA-positive cells is indicated in each panel. FIG. 21B shows a summary of three independent experiments similar to that shown in panel A. Arrow indicates the two 3′ PS modification used in all other figures. FIG. 21C shows the results of the following: cells isolated from mouse spleens were activated for 36 to 42 hours with anti-CD180 (4 μg/ml), high dose LPS (50 μg/ml), or low dose LPS (10 μg/ml) and IL-4 (10 μg/ml). Cells were then electroporated with Mb2Cas12a RNP and HDRT encoding an HA-tag or the VRC26.25 HCDR3, replacing endogenous murine HCDR3s, and incubated in the same conditions for another 18 hours. Cells were then analyzed by flow cytometry with an anti-mouse IgM antibody (horizontal axis) and either an APC-labeled anti-HA tag antibody or an APC-labeled SOSIP trimer. Cells activated with anti-CD180 and electroporated with control templates served as a staining control. Numbers indicate the percentage of successfully edited cells. High dose LPS was used in all other figures except where explicitly stated.

FIGS. 22A-E show the NGS analysis of donor murine B cells. The studies are related to those shown in FIG. 15 and FIG. 16. FIG. 22A shows an analysis of IgH genes of donor CD45.1 cells isolated as in FIG. 15E, except that the distribution of VH1 family genes is shown for donor cells that expressed the native murine HCDR3. Figure shows results combined from donor B cells harvested from two mice for each time point. Note that an enrichment of VH1-64 was observed, similar to the result of successfully edited donor cells. FIGS. 22B and 22C show a time course experiment similar to that shown in FIG. 15A, except that a less immunogenic KLH-conjugated CRF250-v7ds SOSIP was used. Two mice were sacrificed at the indicated time points following each immunization. B cells isolated from spleen and lymph nodes, or from engrafted mice without immunization, were analyzed by flow cytometry. A representative flow figure is shown for each group. Germinal center (GC) B-cells identified as GL7+/CD38-were gated and quantified (top panels). These gated cells were then analyzed for the frequency of CD45.1+, indicating they derived from donor mice. The mean percentage of GC- and CD45.1-positive cells is shown from two mice harvested after each indicated immunization is shown. Donor B cells (CD45.1+) were sorted and pooled for sequencing. Bar graphs in FIG. 22C show the frequency of HCDR3 mutations present in successfully edited clones. The mutations also observed in FIG. 16A are marked with triangles. The first two and last three residues shown derive from murine VH1-family and JH4 segments, respectively, rather than the VRC26.25 HCDR3. FIG. 22D shows the following: at the top, nine individual mutations identified from FIG. 16A are shown beneath the amino acid sequence of the input VRC26.25 HCDR3. Unmodified VRC26.25 or VRC26.25 variants bearing these mutations were expressed as transmembrane antibodies on 293T cell surface and analyzed by flow cytometry with ConM SOSIP proteins (v8.1ds) modified with the CRF250 or BG505 V1V2 regions, as indicated, conjugated to APC. Cells were co-stained with FITC-labeled anti-human Fc. The ratio between these two signals is shown. FIG. 22E shows a figure similar to that in panel C except that the indicated combinations of mutations were analyzed. The neutralization profiles of the highest binding variants (NEE, NER, and NERE) are shown in FIG. 16B and FIG. 16C.

FIGS. 23A-B show designs of SOSIP variants, related to FIG. 17. FIG. 23A shows linear representations of the indicated SOSIP versions (v5, v7ds, v8.1ds, v8.1 and v8.1mut3ds). Constant and variable regions of Env gp120 and the helical repeat (HR) regions of Env gp41 are indicated, mutations shown in blue (E47D, N49E, V65K, E106T, I165L, 432Q, G429R, K500R) indicate modifications from v5 SOSIP sequence to v7 SOSIP, red (F519S, L568D, V570H, R585H) indicates modifications from v7 to v8.1, and green (N302M, T32L, A329P) indicated modification from v8.1 to v8.1mut3. An additional ‘ds’ disulfide bond is indicated with the extra bracket linking I201C to A433C. The positions of “V1V2” region and the “base” region were labeled with HXB2 numbering. These SOSIP variants were generated using the base from BG505, CRF504, or ConM HIV-1 isolates, which in many cases were modified by a V1V2 region from a different isolate. Thus, 16055-ConM-v8.1 indicates a ConM-v8.1 SOSIP modified with the V1V2 region of the 16055 isolate. FIG. 23B shows the sequence of the VIV2 region of Envs used to modified on different bases.

FIGS. 24A-D show in vitro down-selection of SOSIP variants. The studies are related to those shown in FIG. 17 and FIG. 18. FIG. 24A shows the results of the following: the indicated SOSIP variants were characterized for their ability to bind primary murine B cells edited to express the VRC26.25 HCDR3. Each variant is labeled according to the HIV-1 isolate contributing its V1V2 region (BG505, CRF250, ConM), the remainder of the engineered Env ectodomain (BG505, ConM), and the SOSIP version used (v5, v7ds, v8.1, v8.1ds). Protein was produced in Expi293F cells or the same cells lacking the acetylglucosaminyltransferase enzyme (GnTI−). Numbers indicate the percentage of SOSIP-binding cells observed in the indicated gate.

FIG. 24B shows the percentage of SOSIP-binding cells and mean fluorescent intensity of these cells analyzed in panel B are represented. FIG. 24C shows an SEC-purification profile of Expi293F expressed ConM-v8.1ds SOSIPs conjugated on mi3-60mers using a Sephacryl S-400 HR HiPrep 26/60 column. The elution fractions of the SOSIP-mi3 and unconjugated SOSIP are indicated. FIG. 24D shows an SDS-PAGE analysis of the indicated conjugated and unconjugated SOSIPs before and after SEC purification, stained by Coomassie blue. The molecular weight of marker is included. ST2 indicates the presence of a SpyTag2 at the SOSIP C-terminus; SPC3 indicates a SpyCatcher3 domain at the N-terminus of the mi3 60-mer.

FIGS. 25A-F show the optimization of a murine model engrafted with VRC26.25 HCDR3-edited B cells. The studies are related to those shown in FIG. 17 and FIG. 18. FIG. 25A shows the following: 16 μg of the ConMv8.1ds SOSIP trimers conjugated to KLH, the mi3 60-mer, or as a free trimer (none). FIG. 25B shows the following: mi3-conjugates of the 16055-ConM-v8.1ds or BG505-ConM-v8.1ds SOSIP variants were produced in Expi239F cells or the same cell lacking the enzyme GnTI (GnTI−) which adds complex glycans on SOSIP proteins. FIG. 25C shows the results of the following: varying amounts of MPLA adjuvant and protein antigens were tested. Two protein antigens (16055-ConM-v8.1ds and BG505-ConM-v8.1ds) were tested in different ranges of dosage. FIG. 25D shows the results of the following: different ex vivo activation methods (anti-CD180, high-dose LPS) of B cells and number of donor B cells transferred to the recipient mouse were tested. For panel A-D, immunization efficiency was determined by the neutralizing response in mice engrafted with HCDR3-edited B cells. Neutralization assays used sera harvested one week after the second immunization, with the same schedule shown in FIG. 14E. Statistical differences were determined by one-way ANOVA (ns, not significant, *, p<0.05 and **, p<0.01, and ***, p<0.001). FIG. 25E shows the following: mice immunized with 16055-ConM-v8.1ds from FIG. 18C were further characterized. A table summarizing ID₅₀ values of pooled sera from neutralization assays against the indicated pseudoviruses derived from tier 2 HIV-1 isolates from the indicated clades. Sera was collected from mice with or without engraftment of edited B cells one week after each immunization. FIG. 25F shows the following: sera from engrafted mice characterized in panel E was further harvested each month at the time points indicated, and the ID₅₀ of neutralizing sera against the CRF250 pseudovirus was plotted with days.

FIGS. 26A-E show that potent immune responses are elicited by mRNA-LNP expressing SOSIP-TM variants. The studies are related to those in FIG. 19. FIG. 26A shows a linear representation of a SOSIP-TM construct. The membrane proximal region (MPER), the transmembrane domain (TM) and a cytoplasmic domain truncated at residue 712 (CT) are added to the SOSIP constructs shown in FIG. 23A. SOSIP-TM nomenclature is identical to that used for soluble SOSIP proteins. FIG. 26B shows the results of the following: for the experiment in FIG. 19A, mice were sacrificed after two immunizations to monitor the in vivo response of HIV-1 Env-specific donor B cells. Top panel shows flow cytometry analysis of germinal-center B cells (GL7+/CD38−). Bottom panels show the percentage of CD45.1 donor mouse B cells that recognized the HIV-1 Env trimer [SOSIP (+)] within the germinal center. FIG. 26C shows the following: the diameter of LNP particles was monitored by dynamic light scattering to quality control individual LNP preparations. The distribution profile shows mRNA-LNP expressing the indicated SOSIP-TM variants. FIG. 26D shows the results of the following: the ability of sera from these mice to bind to 16055-ConM-v8.1ds SOSIP protein was determined by ELISA. Left bars in each pair indicate pooled sera from 7 immunized mice engrafted with HCDR3-edited B cells, and right bars in each pair indicated pooled sera from 3 mice immunized without engraftment with edited B cells. Each sample was assayed with three technical replicates. Neutralization studies of the same sera are shown in FIG. 19E. FIG. 26E shows the results of the following: competition ELISA assays were performed to evaluate the specificity of antibody responses. Specifically, the indicated sera analyzed in panel D was used to prevent binding of VRC26.25 or a germline form of VRC26.25 to 16055-ConM SOSIP. The percentage of competition was calculated based on the binding of VRC26.25 in the presence of naïve mouse serum. Grey (second and fourth panels) indicates vaccinated mice without engraftment with edited B cells. Significant differences in panels D-E were determined by two-way ANOVA (ns, not significant; *p<0.05; **p<0.01; ***p<0.001; ****p<0.0001).

FIGS. 27A-D show an approach for developing an apex-focused HIV-1 vaccine. This figure is related to studies shown in FIG. 20. FIG. 27A shows an alignment of the HCDR3 sequences of PG9, PG16, VRC26.25 and the VRC26-UCA, each deriving from IGHD3-3, and the open reading frames of four D3 diversity segments encoding YYDX. Homology with the YYDF motif (SEQ ID NO: 42) present in D3-3 are highlighted. Other homologies with D3-3 are also indicated. Tyrosines shown to be sulfated are indicate in a lighter color. FIG. 27B shows previously reported structures of Env trimers complexed with the bnAbs. VRC26.25 (PDB: 6VTT) or PG9 (PDB: 5VJ6, (Wang et al., 2017)) are presented with only YYDF region (SEQ ID NO: 42) of the HCDR3 of these bnAbs shown. Individual Env protomers are indicated in shades of grey. YYDF (SEQ ID NO: 42) contact residues on Env are indicated. YYDF (SEQ ID NO: 42) carbon, oxygen, nitrogen, and sulfur atoms are also shown. FIG. 27C shows the analysis of previously described BCR repertoires from 10 HIV-1 uninfected human donors. The percentage of HCDR3 deriving from the indicated D segment is subtracted from the percentage of the same D segment in the population of ‘apex-like’ HCDR3. A HCDR3 is defined as apex-like if its length is 24 or greater, and it has a tyrosine-sulfation motif 7 or more residues from the HCDR3 amino-terminus and 10 or more residues from its carboxy-terminus. A tyrosine sulfation motif is described as a tyrosine adjacent to an acidic residue without a proximal positive residue. Four D3 segments are enriched in the apex-like population. Each of these bears a YYDX motif. FIG. 27D shows the frequency of long, sulfated ‘apex-like’ HCDR3 from each donor, along with subsets with bearing the indicated motifs, and subsets deriving from the indicated D3 gene segments.

### DETAILED DESCRIPTION

Described herein is a method for rewriting the BCR of mature B cells. This approach is called native-loci editing because the recombined murine heavy- and kappa-chain variable regions are simply replaced by human versions, without displacement or additional regulatory elements. Earlier pioneering studies (8-12) successfully employed an alternative method in which a cassette expressing an exogenous promoter, variable and constant light-chain gene segments, heavy-chain variable segments, and several other regulatory sequences, was introduced into an intron downstream of JH4, the 3′-most mouse JH gene. The native-loci method described herein was compared to one such intron-targeting approach (8) by introducing B cells engineered through each approach into wild-type mice, and then vaccinating these mice with appropriate antigens. Markedly higher neutralization activity in plasma of mice engrafted with native loci-edited B cells was observed, especially following earlier immunizations. Significantly greater rates of somatic hypermutation were also observed in these cells. These high somatic hypermutation (SHM) rates facilitated affinity maturation of BCRs engineered to express the variable chains of the bNAb 10-1074. Robust SHM and further maturation of edited BCRs suggest that native-loci editing does not significantly disrupt development or function of the edited B cell.

Provided herein are methods to directly replace recombined heavy- and kappa-variable genes with those from human antibodies, leaving each locus otherwise unmodified. Compared with prior approaches, the disclosed native-loci editing approach generated more potent neutralizing plasma, more robust somatic hypermutation (SHM), and effective in vivo affinity maturation. Further provided herein are more potent 10-1074 antibody variants generated by the disclosed SHM and affinity maturation.

The disclosed methods may be used to generate human antibody variants that have higher potency (or affinity, or avidity) against a target antigen than the unmutated antibody. The disclosed methods may be used to generate human antibody variants that have higher bioavailability in a mammalian subject, such as a human subject, than the unmutated antibody. The disclosed methods may be used to generate human antibody variants that have greater half-lives than the unmutated antibody.

The present disclosure provides B-cell editing methods. The methods comprise contacting a B cell obtained from a mammalian subject with a homology-directed repair (HDR) template comprising a first sequence encoding heavy chain variable genes of a human antibody, a clustered regularly interspaced short palindromic repeats (CRISPR)-associated protein (Cas), and a guide RNA, wherein the B cell comprises a genomic locus encoding a B cell receptor (BCR), and wherein the guide RNA comprises a sequence having complementarity to the genomic locus; wherein the Cas protein introduces a double-strand DNA break immediately adjacent to a target site in the genomic locus, and the target site is replaced with the first sequence through HDR, thereby generating an engineered BCR; and administering the B cell comprising the engineered BCR to the subject.

The disclosure provides methods of generating improved B-cell receptors, e.g., with greater proliferation, more potent neutralizing sera and more efficient and site-appropriate somatic hypermutation. The disclosure further provides engineered B cells, in particular engineered B cells adapted for SHM in an animal model, such as murine model. Accordingly, provided herein are engineered primary B cells, such as engineered fully mature murine B cells obtained from non-transgenic models. In some embodiments, B cells obtained from a wild-type murine subject comprise an HDR template comprising a first sequence encoding heavy chain variable genes of a human antibody and/or an HDR template comprising a second sequence encoding light chain variable genes of the human antibody, a Cas protein, and a guide RNA, wherein the B cell comprises a genomic locus encoding a BCR.

In some embodiments, the guide RNA comprises a sequence having complementarity to the genomic locus of a BCR. In some embodiments, the guide RNA comprises a sequence having a length of about 10-100 bp, 10-50 bp. 10-40 bp, or 10-30 bp that is complementary to the genomic locus. In some embodiments, the guide RNA has a sequence of 10-30 nucleotides in length that is complementary to the chromosomal target sequences. In some embodiments, the guide RNA has a conserved backbone (or direct-repeat) sequence of about 20 nucleotides. In some embodiments, the length of a guide RNA is dependent on the types of CRISPR effector protein used in the experiment, e.g., Cas9, Cas12a, Cas13. In exemplary embodiments, the guide RNA of the disclosure has a backbone sequence specific for Cas12. In some embodiments, the guide RNA of the disclosure has a backbone sequence specific for Cas9.

In some embodiments, the Cas protein introduces a double-strand DNA break immediately adjacent to at least one target site in the genomic locus. In some embodiments, the at least one target site is replaced with the first sequence and/or the second sequence through HDR, thereby generating an engineered BCR. In some embodiments, the B cell comprising the engineered BCR is administered to the subject. In some embodiments, the heavy chain variable genes of a human antibody comprise the germline VDJ segments. In some embodiments, the light chain variable genes of a human antibody comprise the germline VH segments.

In various embodiments, the disclosed BCR editing methods are performed ex vivo in a B cell obtained from a mouse subject, such as a non-transgenic mouse. In some embodiments, an engineered B cell containing an engineered BCR is generated by any of the disclosed methods. In some embodiments, the engineered B cell is transferred into the mouse subject from which it was obtained. In various embodiments, following this step of adoptive transfer, the BCR is allowed to undergo rapid SHM, class switching, and ultimately, affinity maturation. The rates of diversification (SHM) and results of in vivo selection demonstrated in the Examples of this disclosure surpass existing methods of B cell editing, such as intronic editing methods that disrupt the regulation of B cell development and expansion in response to the antigenic target. As such, in some embodiments, the method provides rates of somatic hypermutation of between 0.5% and 5%, 0.8% and 5%, 1% and 5%, 1.2% and 5%, 1.5% and 5%, 1.8% and 5%, 2% and 5%, 2.5% and 5%, 2.8% and 5%, 3% and 5%, and 3.5% and 5%. In various embodiments, the engineered BCRs of the disclosure are not integrated into the genome of the animal model.

In some embodiments, the engineered B cell is transferred into a subject other than the subject from which the B cell was obtained. In some embodiments, the engineered B cell is transferred into the mouse subject from which it was obtained, and a second mouse subject.

The disclosed methods may further be used to mutate a combinatorial mammalian (e.g., human) antibody library. Such libraries may be silent or coding, based on previous in vivo somatic mutations. For these methods, dozens of variants of BCRs are generated to achieve expression of members of a large combinatorial human antibody library.

The disclosure also provides methods of generating more potent antibodies, e.g., bNAb variants. In some embodiments, the antibodies retain the low polyreactivity. In some embodiments, the antibodies are the HIV-1 neutralizing antibody 10-1074 and variants thereof. In some embodiments, the antibodies have exhibited improved bioavailability as compared to the unmutated antibodies.

### Definitions

The term “administration” or “administering” includes routes of introducing the compound of the invention(s) to a subject to perform their intended function. Examples of routes of administration that may be used include injection (subcutaneous, intravenous, parenterally, intraperitoneally, intrathecal), oral, inhalation, rectal and transdermal. The pharmaceutical preparations may be given by forms suitable for each administration route. For example, these preparations are administered in tablets or capsule form, by injection, inhalation, eye lotion, ointment, suppository, etc. administration by injection, infusion or inhalation; topical by lotion or ointment; and rectal by suppositories. Oral administration is preferred. The injection can be bolus or can be continuous infusion. Depending on the route of administration, the compound of the invention can be coated with or disposed in a selected material to protect it from natural conditions which may detrimentally affect its ability to perform its intended function. The compound of the invention can be administered alone, or in conjunction with either another agent as described above or with a pharmaceutically-acceptable carrier, or both. The compound of the invention can be administered prior to the administration of the other agent, simultaneously with the agent, or after the administration of the agent. Furthermore, the compound of the invention can also be administered in a pro-drug form which is converted into its active metabolite, or more active metabolite in vivo.

As used herein, the term “antibody” refers to a protein that includes at least one immunoglobulin variable domain or immunoglobulin variable domain sequence. For example, an antibody can include a heavy (H) chain variable region (abbreviated herein as VH), and a light (L) chain variable region (abbreviated herein as VL). In another example, an antibody includes two heavy (H) chain variable regions and two light (L) chain variable regions. An antibody can have the structural features of IgA, IgG, IgE, IgD, IgM (as well as subtypes thereof).

The VH and VL regions can be further subdivided into regions of hypervariability, termed “complementarity determining regions” (“CDR”), interspersed with regions that are more conserved, termed “framework regions” (“FR”). The extent of the framework region and CDRs has been precisely defined (see. Kabat. E. A., et al. (1991) Sequences of Proteins of Immunological Interest, Fifth Edition, U.S. Department of Health and Human Services, NIH Publication No. 91-3242, and Chothia. C. et al. (1987) J. Mol. Biol. 196:901-917, see also www.hgmp.mrc.ac.uk). Kabat definitions are used herein. Each VH and VL is typically composed of three CDRs and four FRs, arranged from amino-terminus to carboxy-terminus in the following order: FR1, CDR1, FR2, CDR2, FR3, CDR3, FR4.

The VH or VL chain of the antibody can further include a heavy or light chain constant region, to thereby form a heavy or light immunoglobulin chain, respectively. In one embodiment, the antibody is a tetramer of two heavy immunoglobulin chains and two light immunoglobulin chains, wherein the heavy and light immunoglobulin chains are inter-connected by, e.g., disulfide bonds. In IgGs, the heavy chain constant region includes three immunoglobulin domains, CH1, CH2 and CH3.

The term “monoclonal antibody,” as used herein, refers to a preparation of antibody molecules of single molecular composition. A monoclonal antibody composition displays a single binding specificity and affinity for a particular epitope.

The term “human antibody”, as used herein, is intended to include antibodies having variable regions in which both the framework and CDR regions are derived from human germline immunoglobulin sequences. Furthermore, if the antibody contains a constant region, the constant region also is derived from human germline immunoglobulin sequences. The human antibodies of the invention may include amino acid residues not encoded by human germline immunoglobulin sequences (e.g., mutations introduced by random or site-specific mutagenesis in vitro or by somatic mutation in vivo). However, the term “human antibody”, as used herein, is not intended to include antibodies in which CDR sequences derived from the germline of another mammalian species, such as a mouse, have been grafted onto human framework sequences.

The term “monoclonal antibody” refers to antibodies displaying a single binding specificity which have variable regions in which both the framework and CDR regions are derived from human germline immunoglobulin sequences. In one embodiment, the unmutated human antibodies of the disclosure are human monoclonal antibodies, produced by a hybridoma which includes a B cell obtained from a transgenic nonhuman animal, e.g., a transgenic mouse, having a genome comprising a human heavy chain transgene and a light chain transgene fused to an immortalized cell.

The term “recombinant human antibody”, as used herein, includes all human antibodies that are prepared, expressed, created or isolated by recombinant means, such as (a) antibodies isolated from an animal (e.g., a mouse) that is transgenic or transchromosomal for human immunoglobulin genes or a hybridoma prepared therefrom (described further below), (b) antibodies isolated from a host cell transformed to express the human antibody, e.g., from a transfectoma, (c) antibodies isolated from a recombinant, combinatorial human antibody library, and (d) antibodies prepared, expressed, created or isolated by any other means that involve splicing of human immunoglobulin gene sequences to other DNA sequences. Such recombinant human antibodies have variable regions in which the framework and CDR regions are derived from human germline immunoglobulin sequences. In certain embodiments, however, such recombinant human antibodies can be subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo.

The term “humanized antibody” is intended to refer to antibodies in which CDR sequences derived from the germline of another mammalian species, such as a mouse, have been grafted onto human framework sequences. Additional framework region modifications may be made within the human framework sequences.

The term “antigen-binding fragment” of an antibody (or simply “antibody fragment”), as used herein, refers to one or more fragments of an antibody that retain the ability to specifically bind to an antigen. It has been shown that the antigen-binding function of an antibody can be performed by fragments of a full-length antibody. Such antibody embodiments may also be bispecific, dual specific, or multi-specific formats; specifically binding to two or more different antigens. Multispecific and bispecific antibody constructs are well known in the art and described and characterized in Kontermann (ed.), Bispecific Antibodies, Springer, NY (2011), and Spiess et al., Mol. Immunol. 67(2):96-106 (2015), each of which are incorporated by reference herein.

As used herein, the term “subject” includes any human or nonhuman animal. The term “nonhuman animal” includes all vertebrates. e.g., mammals and non-mammals, such as nonhuman primates, sheep, dogs, cats, horses, cows chickens, amphibians, reptiles, etc. Except when noted, the terms “patient” or “subject” are used interchangeably. Non-human animals includes all vertebrates, e.g., mammals and non-mammals, such as non-human primates, sheep, dogs, cats, cows, horses, chickens, amphibians, and reptiles, although mammals are preferred, such as non-human primates, sheep, dogs, cats, cows and horses.

As used herein, the terms “having affinity for” or “specifically binds to” an antigen or an epitope are well understood in the art. An antibody is said to exhibit “specific binding” if it reacts more frequently, more rapidly, with greater duration and/or with greater affinity with a particular target antigen or epitope than it does with alternative targets. An antibody “specifically binds” to a target ligand or epitope if it binds with greater affinity, avidity, more readily, and/or with greater duration than it binds to other substances. It is also understood with this definition that, for example, an antigen that specifically binds to a first target ligand or antigen may or may not specifically or preferentially bind to a second target ligand or antigen. As such, “specific binding” or “preferential binding” does not necessarily require (although it can include) exclusive binding.

Binding affinity (or binding specificity) can be determined by a variety of methods including equilibrium dialysis, equilibrium binding, gel filtration, ELISA, surface plasmon resonance, or spectroscopy (e.g., using a fluorescence assay). Exemplary conditions for evaluating binding affinity are in HBS-P buffer (10 mM HEPES pH 7.4, 150 mM NaCl. 0.005% (v/v) Surfactant P20). These techniques can be used to measure the concentration of bound binding protein as a function of target protein concentration. The concentration of bound binding protein ([Bound]) is generally related to the concentration of free target protein ([Free]) by the following equation:

[Bound]=[Free]/(K_{D}+[Free])

It is not always necessary to make an exact determination of K_{A} or K_{D} though, since sometimes it is sufficient to obtain a quantitative measurement of affinity, e.g., determined using a method such as ELISA or FACS analysis, is proportional to K_{A} or K_{D}, and thus can be used for comparisons, such as determining whether a higher affinity is. e.g., two-fold higher, to obtain a qualitative measurement of affinity, or to obtain an inference of affinity, e.g., by activity in a functional assay, e.g., an in vitro or in vivo assay.

The term “regulatory element.” as used herein, refers to a region or regions of a nucleic acid sequence that regulates transcription. Exemplary regulatory elements include, but are not limited to, promoters, enhancers, post-transcriptional elements, transcriptional control sequences, and such like.

As used herein, the term “variant” refers to a molecule (e.g. an antibody) having characteristics that deviate from what occurs in nature, e.g., a “variant” is at least about 80% identical, at least about 90% identical, at least about 95% identical, at least about 96% identical, at least about 97% identical, at least about 98% identical, at least about 99% identical, at least about 99.5% identical, or at least about 99.9% identical to the wild type protein. Variants of a protein molecule, e.g. an antibody, may contain modifications to the amino acid sequence (e.g., having 1, 2, 3, 4, 5, 6, 7, 8, 9, 10, 10-15, or 15-20 amino acid substitutions) relative to the wild type protein sequence, which arise from point mutations installed into the nucleic acid sequence encoding the protein. These modifications include chemical modifications as well as truncations, such as truncations at the N- or C-terminus of a protein sequence.

“Percent (%) identity” refers to the extent to which two sequences (nucleotide or amino acid) have the same residue at the same positions in an alignment. For example, “an amino acid sequence is X % identical to SEQ ID NO: Y” refers to % identity of the amino acid sequence to SEQ ID NO: Y and is elaborated as X % of residues in the amino acid sequence are identical to the residues of sequence disclosed in SEQ ID NO: Y. Generally, computer programs are employed for such calculations. Exemplary programs that compare and align pairs of sequences, include ALIGN (Myers and Miller. 1988), FASTA (Pearson and Lipman. 1988; Pearson, 1990) and gapped BLAST (Altschul et al., 1997), BLASTP, BLASTN, or GCG (Devereux et al., 1984).

Typically, the selected sequence and the reference sequence will have at least about 76, 77, 78, 79, 80, 81, 82, 83, 84 or even 85 percent sequence identity, and more preferably, at least about 86, 87, 88, 89, 90, 91, 92, 93, 94, or 95 percent sequence identity. More preferably still, highly homologous sequences often share greater than at least about 96, 97, 98, or 99 percent sequence identity between the selected sequence and the reference sequence to which it was compared. The percentage of sequence identity may be calculated over the entire length of the sequences to be compared, or may be calculated by excluding small deletions or additions which total less than about 25 percent or so of the chosen reference sequence. The reference sequence may be a subset of a larger sequence.

When highly-homologous fragments are desired, the extent of percent identity between the two sequences will be at least about 80%, preferably at least about 85%, and more preferably about 90% or 95% or higher, as readily determined by one or more of the sequence comparison algorithms well-known to those of skill in the art, such as e.g., the FASTA program analysis described by Pearson and Lipman (1988) and blastn computer program based on the algorithm of Brutlag et al. (Comp. App. Biosci. 6:237-245 (1990). A preferred method for determining the best overall match between a query sequence (e.g., a sequence of the present disclosure) and a subject sequence, also referred to as a global sequence alignment, can be determined using the FASTA or blastn. In a sequence alignment the query and subject sequences are either both nucleotide sequences or both amino acid sequences. The result of said global sequence alignment is expressed as percent identity. Preferred parameters used in a FASTA amino acid alignment are: Matrix=PAM 0, k-tuple=2, Mismatch Penalty=1. Joining Penalty=20, Randomization Group Length=0, Cutoff Score=1, Window Size=sequence length, Gap Penalty=5, Gap Size Penalty=0.05. Window Size=500 or the length of the subject amino acid sequence, whichever is shorter. Whether a nucleotide is matched/aligned is determined by results of the FASTA sequence alignment. This percentage is then subtracted from the percent identity, calculated by the above FASTA program using the specified parameters, to arrive at a final percent identity score. This final percent identity score is what is used for the purposes of the present disclosure.

As used herein. “AAV” is adeno-associated virus, and may be used to refer to the naturally occurring wild-type virus itself or derivatives thereof. The term covers all subtypes, serotypes and pseudotypes, and both naturally occurring and recombinant forms, except where required otherwise. As used herein, the term “serotype” refers to an AAV which is identified by and distinguished from other AA Vs based on capsid protein reactivity with defined antisera, e.g., serotypes including AAV1, AAV2, AAV3. AAV4, AAV5, AAV6, AAV7, AAV8, AAV9, AAV10, and AAV11. For example, serotype AAV6 is used to refer to an AAV which contains capsid proteins encoded from the cap gene of AAV6 and a genome containing 5′ and 3′ ITR sequences from the same AAV6 serotype. Pseudotyped AAV refers to an AAV that contains capsid proteins from one serotype and a viral genome including 5′-3′ ITRs of a second serotype. Pseudotyped rAAV would be expected to have cell surface binding properties of the capsid serotype and genetic properties consistent with the second serotype. The abbreviation “rAAV” refers to recombinant adeno-associated viral particle or a recombinant AAV vector (or “rAAV vector”). An “AAV virus” or “AAV viral particle” refers to a viral particle composed of at least one AA V capsid protein (preferably by all of the capsid proteins of a wild-type AA V) and an encapsidated polynucleotide. If the particle comprises a heterologous polynucleotide (i.e., a polynucleotide other than a wild-type AAV genome such as a transgene to be delivered to a mammalian cell), it is typically referred to as “rAAV”.

The term “treating” or “alleviating” includes the administration of compounds or agents (e.g., pharmaceutical compositions comprising an antibody variant) to a subject to prevent or delay the onset of the symptoms, complications, or biochemical indicia of a disease, alleviating the symptoms or arresting or inhibiting further development of the disease, condition, or disorder, such as an infectious disease.

Subjects in need of treatment include those already suffering from the disease or disorder as well as those being at risk of developing the disorder. Treatment may be prophylactic (to prevent or delay the onset of the disease, or to prevent the manifestation of clinical or subclinical symptoms thereof) or therapeutic suppression or alleviation of symptoms after the manifestation of the disease.

As used herein, a “vector” is a nucleic acid with or without a carrier that can be introduced into a cell. Vectors capable of directing the expression of genes encoding for one or more polypeptides are referred to as “expression vectors”. Examples of vectors suitable for the invention include, e.g., viral vectors, plasmid vectors, liposomes and other gene delivery vehicles.

As used herein, “B cells” are B lymphocytes, a type of white blood cell of the lymphocyte subtype and engineered B-cells. Examples of B-cells include plasmablast, plasma cells, lymphoplasmacytoid cells, memory B cells, B-2 cells, B-1 cells, regulatory B-cells.

As used herein, “somatic hypermutation” (SHM) refers to the increased mutation of B-cell receptor loci gene regions encoding variable regions of the light and heavy chains in B lymphocytes following antigen stimulation.

### B-Cell Receptors and Generated Antibodies

The B-cell receptor or BCR is a transmembrane receptor protein located on the outer surface of B cells. The receptor's binding moiety is composed of a membrane-bound antibody.

The disclosed methods may be applied to promote the affinity maturation of existing antibodies (such as commercial and/or FDA-approved monoclonal antibodies) or newly discovered antibodies. The disclosed methods involve contacting a B cell obtained from a mammalian subject with an HDRT comprising a first sequence encoding heavy chain variable genes of a human antibody and a second sequence encoding light chain variable genes of a human antibody, a Cas protein, and a guide RNA, wherein the B cell comprises a genomic locus encoding a BCR. In some embodiments, the heavy chain is a VH sequence, and the light chain is a VK sequence.

In some embodiments, the heavy chain of the human antibody of the disclosed methods comprises a heavy-chain complementarity-determining region 3 (HC-CDR3).

In some embodiments, the antigenic target is a soluble protein antigen, a transmembrane protein antigen, or a viral antigen. In some embodiments, the antigenic target is an HIV antigen. In some embodiments, the antibody is the HIV-1 bNab 10-1074, or a variant thereof. In some embodiments, the heavy chain is a VH sequence, and the light chain is a VK sequence of 10-1074.

In some embodiments, the antigenic target is MINCLE, CCR5, SLC6A14, or alpha-dystroglycan. In some embodiments, the antigenic target is MINCLE.

The B cells produced by the methods of the invention are engineered to secrete a variant of a therapeutic monoclonal antibody. Therapeutic monoclonal antibodies are well known in the art and include, for example, 3F8,8H9, Abagovomab, Abciximab, Abrilumab, Actoxumab, Adalimumab, Adecatumumab, Aducanumab, Afelimomab, Afutuzumab, Alacizumab pegol, ALD518. Alemtuzumab, Alirocumab, Altumomab pentetate, Amatuximab, Anatumomab mafenatox, Anifrolumab, Anrukinzumab, (=IMA-638), Apolizumab, Arcitumomab, Aselizumab. Atinumab, Atlizumab (=tocilizumab), Atorolimumab, Bapineuzumab, Basiliximab, Bavituximab, Bectumomab, Belimumab, Benralizumab, Bertilimumab, Besilesomab, Bevacizumab, Bezlotoxumab, Biciromab, Bimagrumab, Bivatuzumab mertansine, Blinatumomab, Blosozumab, Brentuximab vedotin, Briakinumab, Brodalumab, Canakinumab, Cantuzumab mertansine, Cantuzumab ravtansine. Caplacizumab, Capromab pendetide, Carlumab, Catumaxomab, CC49, cBR96-doxorubicin immunoconjugate, Cedelizumab, Certolizumab pegol, Cetuximab, Ch.14.18, Citatuzumab bogatox, Cixutumumab, Clazakizumab, Clenoliximab, Clivatuzumab tetraxetan, Conatumumab, Concizumab, Crenezumab, CR6261, Dacetuzumab, Daclizumab, Dalotuzumab, Daratumumab, Demcizumab, Denosumab, Detumomab, Dinutuximab, Diridavumab, Dorlimomab aritox, Drozitumab, Duligotumab. Dupilumab, Durvalumab, Dusigitumab, Ecromeximab, Eculizumab, Edobacomab, Edrecolomab, Efalizumab, Efungumab, Eldelumab, Elotuzumab, Elsilimomab, Emibetuzumab, Enavatuzumab, Enfortumab vedotin, Enlimomab pegol, Enokizumab, Enoticumab, Ensituximab, Epitumomab cituxetan. Epratuzumab, Erlizumab, Ertumaxomab, Etaracizumab, Etrolizumab, Evinacumab, Evolocumab, Exbivirumab, Fanolesomab, Faralimomab, Farletuzumab, Fasinumab, FBTA05, Felvizumab, Fezakinumab, Ficlatuzumab, Figitumumab, Flanvotumab, Fletikumab, Fontolizumab, Foralumab, Foravirumab, Fresolimumab, Fulranumab, Futuximab, Galiximab, Ganitumab, Gantenerumab, Gavilimomab, Gemtuzumab ozogamicin, Gevokizumab, Girentuximab, Glembatumumab vedotin, Golimumab, Gomiliximab, Guselkumab, Ibalizumab, Ibritumomab tiuxetan, Icrucumab, Igovomab, IMAB362, Imciromab, Imgatuzumab, Inclacumab, Indatuximab ravtansine, Infliximab, Intetumumab, Inolimomab, Inotuzumab ozogamicin, Ipilimumab, Iratumumab, Itolizumab, Ixekizumab, Keliximab, Labetuzumab, Lambrolizumab, Lampalizumab, Lebrikizumab, Lemalesomab, Lerdelimumab, Lexatumumab, Libivirumab, Lifastuzumab vedotin, Ligelizumab, Lintuzumab, Lirilumab, Lodelcizumab, Lorvotuzumab mertansine, Lucatumumab, Lulizumab pegol, Lumiliximab, Mapatumumab, Margetuximab, Maslimomab, Mavrilimumab, Matuzumab, Mepolizumab, Metelimumab, Milatuzumab, Minretumomab, Mitumomab, Mogamulizumab, Morolimumab, Motavizumab, Moxetumomab pasudotox, Muromonab-CD3, Nacolomab tafenatox, Namilumab, Naptumomab estafenatox, Narnatumab, Natalizumab, Nebacumab, Necitumumab, Nerelimomab, Nesvacumab, Nimotuzumab, Nivolumab, Nofetumomab merpentan, Obiltoxaximab, Ocaratuzumab, Ocrelizumab, Odulimomab, Ofatumumab, Olaratumab, Olokizumab, Omalizumab, Onartuzumab, Ontuxizumab, Oportuzumab monatox, Oregovomab, Orticumab, Otelixizumab, Otlertuzumab, Oxelumab, Ozanezumab, Ozoralizumab, Pagibaximab, Palivizumab, Panitumumab, Pankomab, Panobacumab, Parsatuzumab, Pascolizumab, Pateclizumab, Patritumab, Pembrolizumab, Pemtumomab, Perakizumab, Pertuzumab, Pexelizumab, Pidilizumab. Pinatuzumab vedotin, Pintumomab, Placulumab, Polatuzumab vedotin, Ponezumab, Priliximab, Pritoxaximab, Pritumumab, PRO 140, Quilizumab, Racotumomab, Radretumab, Rafivirumab, Ramucirumab, Ranibizumab, Raxibacumab, Regavirumab, Reslizumab, Rilotumumab, Rituximab, Robatumumab, Roledumab, Romosozumab, Rontalizumab, Rovelizumab, Ruplizumab, Samalizumab, Sarilumab, Satumomab pendetide, Secukinumab, Seribantumab, Setoxaximab, Sevirumab, Sibrotuzumab, SGN-CD19A, SGN-CD33A, Sifalimumab, Siltuximab, Simtuzumab, Siplizumab, Sirukumab, Sofituzumab vedotin, Solanezumab, Solitomab, Sonepcizumab, Sontuzumab, Stamulumab, Sulesomab, Suvizumab, Tabalumab, Tacatuzumab tetraxctan, Tadocizumab, Talizumab, Tanczumab, Taplitumomab paptox, Tarextumab, Tefibazumab, Telimomab aritox, Tenatumomab, Teneliximab, Teplizumab, Teprotumumab, TGN1412, Ticilimumab (=tremelimumab), Tildrakizumab, Tigatuzumab, TNX-650, Tocilizumab (=atlizumab), Toralizumab, Tositumomab, Tovetumab, Tralokinumab, Trastuzumab, TRBS07, Tregalizumab, Tremelimumab, Tucotuzumab celmoleukin, Tuvirumab, Ublituximab, Urelumab, Urtoxazumab, Ustekinumab, Vantictumab, Vapaliximab, Varlilumab, Vatelizumab, Vedolizumab, Veltuzumab, Vepalimomab, Vesencumab, Visilizumab, Volociximab, Vorsetuzumab mafodotin, Votumumab, Zalutumumab Zanolimumab, Zatuximab, Ziralimumab, and Zolimomab.

The disclosed (therapeutic) antibody variants may have affinity for, or be specific for, any antigen that can be bound by a cell surface antibody. The disclosed (therapeutic) antibody variants may have affinity for, or be specific for, an antigen in a viral protein, such as an envelop (Env) protein. The disclosed antibody variants may have affinity for an antigen in a transmembrane protein. The disclosed antibody variants may have affinity for a membrane-bound protein. The disclosed antibody variants may have affinity for a soluble protein. In some embodiments, the disclosed antibody variants have affinity for an antigen in HIV Env protein. In some embodiments, the disclosed antibody variants have affinity for an antigen in PCSK9 protein.

The disclosed therapeutic antibodies may have affinity for, or be specific for, one or more antigens in the following proteins: TNF-α, IGHE, IL-1, IL-1β, IL-2, IL-4, IL-5, IL-6, IL-6R, IL-9, IL-12 IL-13, IL-17A, IL-20, IL-22, IL-23, IL-25, BAFF, RANKL, Integrin-α4, IL-6R, VEGF-A, VEGFR1, VEGFR2, EGFR, HER2, HER3, CA125, integrin α4β7, integrin α7β7, interferon α/β receptor, CXCR4, CD2, CD3, CD4, CD5, CD6, CD19, CD20, CD22, CD23, CD25, CD27, CD28, CD30, CD33, CD37, CD38, CD40, CD41, CD44, CD51, CD52, CD56, CD70, CD74, CD79B, CD80, CD125, CD137, CD140a, CD147, CD152, CD154, CD200, CD221, CCR4, CCR5, gp120, angiopoietin 3, PCSK9, HNGF, HGF, GD2, GD3, C5, FAP, ICAM-1, LFA-1, interferon alpha, interferon gamma, interferon gamma-induced protein, SLAMF7, HHGFR, TWEAK receptor, NRP1, EpCAM, CEA, CEA-related antigen mesothelin, MUC1, IGF-1R, TRAIL-R2, DR5, DLL4, VWF, MCP-1, β-amyloid, phosphatidyl serine, Rhesus factor, CCL11, CXCR4 NARP-1, RTN4, ACVR2B, SOST, NOGO-A, sclerostin, TGF-β, TGF-BR1, NGF, LTA, AOC3, ITGA2, GM-CSF, GM-CSF receptor, oxLDL, LOXL2, RON, KIR2D, PD-1, PD-L1, CTLA-4, LAG-3, TIM-3, BTLA, MINCLE, CCR5. SLC6A14, and alpha-dystroglycan, episialin, myostatin, hemagglutinin, rabies virus glycoprotein, or cytomegalovirus glycoprotein B. The disclosed therapeutic antibodies may have affinity for, or be specific for, one or more antigens expressed by one or more of the following pathogens: anthrax avian influenza, influenza A, hepatitis A virus, hepatitis B virus, hepatitis C virus, respiratory syncytial virus, Tuberculosis, Ebola, Staphylococcus aureus, SARS, MERS, RSV, malaria, HPV, HSV, or HIV. In some embodiments, the antigen is expressed by HIV-1.

### Genomic Editing Tools

Gene editing, or genome editing, is a type of genetic engineering in which DNA is inserted, replaced, or removed from a genome using nucleases. The nucleases may be artificially engineered. Alternately, the nucleases may be found in nature. The nucleases create specific double-stranded breaks (DSBs) at desired locations in the genome. The cell's endogenous repair mechanisms subsequently repairs the induced break(s) by natural processes, such as homologous recombination (HR) and non-homologous end-joining (NHEJ). Nucleases include, for example. Zinc Finger Nucleases (ZFNs), Transcription Activator-Like Effector Nucleases (TALENs), CRISPR, (e.g., the CRISPR/Cas system), and engineered meganuclease re-engineered homing endonucleases. CRISPR nucleases include, for example, a Cas nuclease, a Cpf1 nuclease, a C2c1 nuclease, a C2c3 nuclease, and a C2c3 nuclease.

Described herein are compositions comprising a DNA-binding nuclease that specifically binds to a target site in any B cell gene. In preferred embodiments, the gene is an immunoglobulin gene, a gene that encodes a protein that enhances antigen presentation, a gene that encodes a protein that suppresses antigen presentation, a gene that includes a sequence that is related to antibody retention or secretion, a gene that encodes a cytokine, a gene that promotes differentiation into a memory B cell, a gene that promotes differentiation into a plasma cell, or a gene that promotes trafficking of a B cell to a lymphoid organ (e.g., lymph node, spleen, bone marrow). The disclosed nucleases may mediate homology-directed repair (HDR).

In preferred embodiments, the DNA-binding nuclease comprises a CRISPR/Cas system. The CRISPR (clustered regularly interspaced short palindromic repeats) locus, which encodes RNA components of the system, and the Cas (CRISPR-associated) locus, which encodes proteins (Jansen et al., 2002. Mol. Microbiol. 43: 1565-1575; Makarova et al., 2002. Nucleic Acids Res. 30:482-496; Makarova et al., 2006. Biol. Direct 1:7; Haft et al., 2005. PLoS Comput. Biol. 1: e60) make up the gene sequences of the CRISPR/Cas nuclease system. CRISPR loci in microbial hosts contain a combination of CRISPR-associated (Cas) genes as well as non-coding RNA elements capable of programming the specificity of the CRISPR-mediated nucleic acid cleavage.

The Type II CRISPR is one of the most well characterized systems and carries out targeted DNA double-strand breaks in four sequential steps. First, two non-coding RNAs, the pre-crRNA array and tracrRNA, are transcribed from the CRISPR locus. Second, tracrRNA hybridizes to the repeat regions of the pre-crRNA and mediates the processing of pre-crRNA into mature crRNAs containing individual spacer sequences. Third, the mature crRNA:tracrRNA complex directs Cas9 to the target DNA via Watson-Crick base-pairing between the spacer on the crRNA and the protospacer on the target DNA next to the protospacer adjacent motif (PAM), an additional requirement for target recognition. Finally, Cas9 mediates cleavage of target DNA to create a double-stranded break within the protospacer. Activity of the CRISPR/Cas system comprises of three steps: (i) insertion of alien DNA sequences into the CRISPR array to prevent future attacks, in a process called “adaptation”, (ii) expression of the relevant proteins, as well as expression and processing of the array, followed by (iii) RNA-mediated interference with the alien nucleic acid. Thus, in the bacterial cell, several of the so-called “Cas” proteins are involved with the natural function of the CRISPR/Cas system and serve roles in functions such as insertion of the alien DNA.

Accordingly, Cas proteins are provided for use in any of the disclosed genomic HDR editing methods. In some embodiments, the Cas protein is a Cas9. In some embodiments, the Cas protein is a Cas 12a protein. In some embodiments, the Cas protein is a Cas 13 protein.

In some embodiments, proteins comprising “derivatives” or “variants” of a Cas protein are provided. For example, in some embodiments, a Cas variant comprises one of two Cas9 domains (or Cas12a domains, or Cas13 domains): (1) the gRNA binding domain of Cas9; or (2) the DNA cleavage domain of Cas9. In some embodiments, proteins comprising Cas9 or fragments thereof are referred to as “Cas9 variants.” A Cas9 variant shares homology to Cas9, or a fragment thereof. For example a Cas9 variant is at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to wild type Cas9. In some embodiments, the Cas9 variant may have 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, 50 or more amino acid changes compared to wild type Cas9. In some embodiments, the Cas9 variant comprises a fragment of Cas9 (e.g., a gRNA binding domain or a DNA-cleavage domain), such that the fragment is at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to the corresponding fragment of wild type Cas9. In some embodiments, the fragment is is at least 30%, at least 35%, at least 40%, at least 45%, at least 50%, at least 55%, at least 60%, at least 65%, at least 70%, at least 75%, at least 80%, at least 85%, at least 90%, at least 95% identical, at least 96%, at least 97%, at least 98%, at least 99%, or at least 99.5% of the amino acid length of a corresponding wild type Cas9.

The disclosed native loci editing methods involve the use of guide RNAs (gRNAs) to achieved HDR-directed editing. As such, the methods also involve the introduction of a guide RNA such as a single-guide RNAs (sgRNA) into the cell or the animal model. The guide RNAs (sgRNAs) include nucleotide sequences that are complementary to the target chromosomal DNA. The sgRNAs can be, for example, engineered single chain guide RNAs that comprise a crRNA sequence (complementary to the target DNA sequence) and a common tracrRNA sequence, or as crRNA-tracrRNA hybrids. The sgRNAs can be introduced into the cell or the organism as a DNA (with an appropriate promoter), as an in vitro transcribed RNA, or as a synthesized RNA. In some embodiments, the guide RNA is between 15 and 100 nucleotides long and comprises a sequence of at least 10 contiguous nucleotides that is complementary to a genomic (coding) target sequence in a BCR. In some embodiments, the guide RNA is about 100, about 200, about 250, about 300, about 400, or more than about 400 nucleotides long. In some embodiments, the guide RNA is about 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides long. In some embodiments, the guide RNA comprises a sequence of 15, 16, 17, 18, 19, 20, 21, 22, 23, 24, 25, 26, 27, 28, 29, 30, 31, 32, 33, 34, 35, 36, 37, 38, 39, or 40 contiguous nucleotides that is complementary to the target sequence in the BCR. In some embodiments, the 3′ end of the target sequence is immediately adjacent to a protospacer-adjacent motif (PAM) sequence (such as the canonical PAM sequence, NGG).

Researchers have invested intense effort to increase the efficiency of HDR and suppress NHEJ. For example, a small-molecule inhibitor of ligase IV, an essential enzyme in the NHEJ pathway, has been shown to increase HDR efficiency. However, this strategy is challenging in post-mitotic cells, which typically down-regulate HDR, and its therapeutic relevance is limited by the potential risks of inhibiting ligase IV in non-target cells. Enhanced HDR efficiency can also be achieved by the timed delivery of Cas9-guide RNA complexes into chemically synchronized cells, as HDR efficiency is highly cell-cycle dependent. Such an approach, however, is limited to research applications in cell culture since synchronizing cells is highly disruptive. Despite these developments, current strategies to replace point mutations using HDR in most contexts are very inefficient (typically ˜0.1 to 5%), especially in unmodified, non-dividing cells. In addition, HDR competes with NHEJ during the resolution of double-stranded breaks, and indels are generally more abundant outcomes than gene replacement. These observations highlight the need to develop alternative approaches to install specific modifications in genomic DNA that do not rely on creating double-stranded DNA breaks. A small-molecule inhibitor of ligase IV, an essential enzyme in the NHEJ pathway, has been shown to increase HDR efficiency. However, this strategy is challenging in post-mitotic cells, which typically down-regulate HDR, and its therapeutic relevance is limited by the potential risks of inhibiting ligase IV in non-target cells. Enhanced HDR efficiency can also be achieved by the timed delivery of Cas9-guide RNA complexes into chemically synchronized cells, as HDR efficiency is highly cell-cycle dependent. Such an approach, however, is limited to research applications in cell culture since synchronizing cells is highly disruptive. In some cases, it is possible to design HDR templates such that the product of successful HDR contains mutations in the PAM sequence and therefore is no longer a substrate for subsequent Cas9 modification, increasing the overall yield of HDR products, although such an approach imposes constraints on the product sequences. Recently, this strategy has been coupled to the use of ssDNA donors that are complementary to the non-target strand and high-efficiency ribonucleoprotein (RNP) delivery to substantially increase the efficiency of HDR, but even in these cases the ratio of HDR to NHEJ outcomes is relatively low (<2).

In any of the disclosed CRISPR HDR methods, the editing takes about 48 hours. 50 hours. 72 hours, 84 hours, or 96 hours to complete. In any of the disclosed CRISPR HDR methods, the editing takes about 3 days to complete.

The homology arms of the HDR methods of the disclosure may be delivered to the animal subject by a recombinant AAV (rAAV) particle or virion. The rAAV particle of the disclosed methods, may be of any AAV serotype, including any derivative or pseudotype (e.g., 1, 2, 3, 4, 5, 6, 7, 8, 9, 10, 2/1, 2/5, 2/6, 2/8, 2/9, 3/1, 3/5, 3/8, or 3/9). As used herein, the serotype of an rAAV viral vector (e.g., an rAAV particle) refers to the serotype of the capsid proteins of the recombinant virus. In some embodiments, the rAAV particle is not AAV2. In some embodiments, the rAAV particle is not AAV8. Non-limiting examples of derivatives and pseudotypes include rAAV2/1, rAAV2/5, rAAV2/6, rAAV2/8, rAAV2/9, AAV2-AAV3 hybrid, AAVrh.10, AAVrh.74, AAVhu.14, AAV3a/3b, AAVrh32.33, AAV-HSC15, AAV-HSC17, AAVhu.37, AAVrh.8, CHt-P6, AAV2.5, AAV6.2, AAV218, AAV-HSC15/17, AAVM41, AAV9.45, AAV6(Y445F/Y731F), AAV2.5T, AAV-HAE1/2, AAV clone 32/83, AAVShH10, AAV2 (Y→F), AAV8 (Y733F), AAV2.15, AAV2.4, AAVM41, AAV-DJ and AAVr3.45. These AAV serotypes and derivatives/pseudotypes, and methods of producing such derivatives/pseudotypes are known in the art (see. e.g., Mol Ther. 2012 April; 20(4): 699-708). The AAV vector toolkit: poised at the clinical crossroads. Asokan A1, Schaffer D V, Samulski R J.). In some embodiments, the rAAV particle is a pseudotyped rAAV particle, which comprises (a) a nucleic acid segment comprising ITRs from one serotype (e.g., AAV2, AAV3) and (b) a capsid comprised of capsid proteins derived from another serotype (e.g., AAV1, AAV2, AAV3, AAV4, AAV5, AAV6, AAV7, AAV8, AAV9, or AAV10). Methods for producing and using pseudotyped rAAV vectors are known in the art (see, e.g., Duan et al., J. Virol., 75:7662-7671, 2001; Halbert et al., J. Virol., 74:1524-1532, 2000; Zolotukhin et al., Methods, 28:158-167, 2002; and Auricchio et al., Hum. Molec. Genet., 10:3075-3081, 2001).

Additional serotypes of the rAAV capsids disclosed herein include capsids include AAV2, AAV6 and capsids derived from AAV2 and AAV6. In addition, such capsids include AAV7m8, AAV2/2-MAX, AAVSHh10Y, AAV3, AAV3b, AAVLK03, AAV7BP2, AAV1 (E531K), AAV6 (D532N), AAV6-3pmut and AAV2G9. In some embodiments, the homology arms are delivered in an AAV6 capsid. In some embodiments, the homology arms are delivered in an AAV-DJ capsid.

The AAV-DJ capsid is described in Grimm et al., J. Virol., 2008, 5887-5911 and Katada et al., (2019) Evaluation of AAV-DJ vector for retinal gene therapy, PeerJ 7: e6317 each of which is herein incorporated by reference. The AAV-DJ comprises the insertion of 7 amino acids into the HSPG binding domain of the AAV2 capsid and has high expression efficiency in Muller cells following intravitreal injection. The AAV7m8 capsid, which is closely related to AAV-DJ, is described in Dalkara et al. Sci Transl Med. 2013; 5(189):189ra76, herein incorporated by reference.

### Pharmaceutical Compositions and Methods of Administration

In another aspect, the present invention provides a composition, e.g., a pharmaceutical composition, containing one or a combination of monoclonal antibodies, or antigen-binding portion(s) thereof, generated by any of the disclosed methods, formulated together with a pharmaceutically acceptable agent. Such compositions may include one or a combination of (e.g., two or more different) antibodies, or immunoconjugates or bispecific molecules of the invention. For example, a pharmaceutical composition of the invention can comprise a combination of antibodies (or immunoconjugates or bispecific antibodies) that bind to different epitopes on the target antigen or that have complementary activities.

Further provided herein are pharmaceutical compositions comprising an engineered primary B cell in accordance with the disclosure. Also provided herein are pharmaceutical compositions comprising a population of engineered primary B cells.

Pharmaceutical compositions of the invention also can be administered in combination therapy, i.e., combined with other agents. For example, the combination therapy can include an anti-PD-1 antibody of the present invention combined with at least one other anti-inflammatory or immunosuppressant agent. Examples of therapeutic agents that can be used in combination therapy are described in greater detail below in the section on uses of the antibodies of the invention.

As used herein. “pharmaceutically acceptable agent” includes any and all carriers, buffers, solvents, dispersion media, coatings, antibacterial and antifungal agents, isotonic and absorption delaying agents, and the like that are physiologically compatible. Preferably, the agent is suitable for intravenous, intramuscular, subcutaneous, parenteral, spinal or epidermal administration (e.g., by injection or infusion). Depending on the route of administration, the antibody may be coated in a material to protect the compound from the action of acids and other natural conditions that may inactivate the compound.

The pharmaceutical compounds of the invention may include one or more pharmaceutically acceptable salts. A “pharmaceutically acceptable salt” refers to a salt that retains the desired biological activity of the parent compound and does not impart any undesired toxicological effects (see e.g., Berge. S. M., et al. (1977) J. Pharm. Sci. 66:1-19). Examples of such salts include acid addition salts and base addition salts. Acid addition salts include those derived from nontoxic inorganic acids, such as hydrochloric, nitric, phosphoric, sulfuric, hydrobromic, hydroiodic, phosphorous and the like, as well as from nontoxic organic acids such as aliphatic mono- and dicarboxylic acids, phenyl-substituted alkanoic acids, hydroxy alkanoic acids, aromatic acids, aliphatic and aromatic sulfonic acids and the like. Base addition salts include those derived from alkaline earth metals, such as sodium, potassium, magnesium, calcium and the like, as well as from nontoxic organic amines, such as N,N′-dibenzylethylenediamine, N-methylglucamine, chloroprocaine, choline, diethanolamine, ethylenediamine, procaine and the like.

A pharmaceutical composition of the invention also may include a pharmaceutically acceptable anti-oxidant. Examples of pharmaceutically acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like. Examples of suitable aqueous and nonaqueous carriers that may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of presence of microorganisms may be ensured both by sterilization procedures, supra, and by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

Pharmaceutically acceptable agents include sterile aqueous solutions or dispersions and sterile powders for the extemporaneous preparation of sterile injectable solutions or dispersion. The use of such media and agents for pharmaceutically active substances is known in the art. Except insofar as any conventional media or agent is incompatible with the active compound, use thereof in the pharmaceutical compositions of the invention is contemplated. Supplementary active compounds can also be incorporated into the compositions.

Therapeutic compositions typically must be sterile and stable under the conditions of manufacture and storage. The composition can be formulated as a solution, microemulsion, liposome, or other ordered structure suitable to high drug concentration. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. In many cases, it will be preferable to include isotonic agents, for example, sugars, polyalcohols such as mannitol, sorbitol, or sodium chloride in the composition. Prolonged absorption of the injectable compositions can be brought about by including in the composition an agent that delays absorption, for example, monostearate salts and gelatin.

Sterile injectable solutions can be prepared by incorporating the active compound in the required amount in an appropriate solvent with one or a combination of ingredients enumerated above, as required, followed by sterilization microfiltration. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying (lyophilization) that yield a powder of the active ingredient plus any additional desired ingredient from a previously sterile-filtered solution thereof.

The compositions described herein may be administered locally or systemically. In certain embodiments, administration will be parenteral administration. In certain embodiments, the pharmaceutical composition is administered subcutaneously, and in certain embodiments intravenously. Preparations for parenteral administration include sterile aqueous or non-aqueous solutions, suspensions, and emulsions.

In certain embodiments, a therapeutically effective amount of active component is in the range of 0.1 mg/kg to 100 mg/kg, e.g., 1 mg/kg to 100 mg/kg, 1 mg/kg to 50 mg/kg, 1 mg/kg to 40 mg/kg, 1 mg/kg to 30 mg/kg, 1 mg/kg to 20 mg/kg. 1 mg/kg to 10 mg/kg, 1 mg/kg to 5 mg/kg, 50 mg/kg, 40 mg/kg, 30 mg/kg, 20 mg/kg, 10 mg/kg, 7.5 mg/kg, 5 mg/kg, or 2.5 mg/kg. The amount administered will depend on variables such as the type and extent of disease or indication to be treated, the overall health of the subject, the in vivo potency of the active component, the pharmaceutical formulation, and the route of administration. The initial dosage can be increased beyond the upper level in order to rapidly achieve the desired blood-level or tissue level.

Alternatively, the initial dosage can be smaller than the optimum, and the daily dosage may be progressively increased during the course of treatment. Human dosage can be optimized, e.g., in a conventional Phase I dose escalation study designed to run from 0.5 mg/kg to 30 mg/kg. Dosing frequency can vary, depending on factors such as route of administration, dosage amount, serum half-life of the antibody, and the disease being treated.

Exemplary dosing frequencies are once per day, once per week and once every two weeks. An exemplary route of administration is parenteral, e.g., intravenous infusion. In certain embodiments, a protein or expression vector disclosed herein is lyophilized, and then reconstituted in buffered saline, at the time of administration.

### Therapeutic Uses

The proteins, expression vectors, compositions and methods disclosed herein can be used to treat human immunodeficiency virus (HIV) infection in a subject. The invention provides a method of treating a HIV infection in a subject. The method comprises administering to the subject an effective amount of a protein, expression vector or pharmaceutical composition disclosed herein, either alone or in a combination with another therapeutic agent, to treat the HIV infection in the subject. The invention also provides a method of blocking the entry of HIV into a host cell, e.g., a human host cell. The method comprises exposing the host cell to an effective amount of a protein, expression vector or pharmaceutical composition disclosed herein, either alone or in a combination with another therapeutic agent, to block the entry of HIV into the host cell. The invention also provides a method of causing the killing of a host cell, e.g., a human host cell, infected with HIV. The method comprises exposing the host cell to an effective amount of a protein, expression vector or pharmaceutical composition disclosed herein, either alone or in a combination with another therapeutic agent, to cause the killing of the infected host cell. The invention also provides a method of causing the inactivation of a viral particle, e.g., an HIV viral particle.

The method comprises exposing the viral particle to an effective amount of an antibody, expression vector or pharmaceutical composition disclosed herein, either alone or in a combination with another therapeutic agent, to cause the inactivation of the HIV viral particle. The invention also provides a method of clearing virus particles from the plasma of a subject. e.g., HIV virus particles. The method comprises exposing the subject to an effective amount of a protein, expression vector or pharmaceutical composition disclosed herein, either alone or in a combination with another therapeutic agent, to clear virus particles from the plasma of a subject.

The methods and compositions described herein can be used alone or in combination with other therapeutic agents and/or modalities. The term administered “in combination.” as used herein, is understood to mean that two (or more) different treatments are delivered to the subject during the course of the subject's affliction with the disorder, such that the effects of the treatments on the patient overlap at a point in time. In certain embodiments, the delivery of one treatment is still occurring when the delivery of the second begins, so that there is overlap in terms of administration. This is sometimes referred to herein as “simultaneous” or “concurrent delivery.” In other embodiments, the delivery of one treatment ends before the delivery of the other treatment begins. In certain embodiments of either case, the treatment is more effective because of combined administration. For example, the second treatment is more effective. e.g., an equivalent effect is seen with less of the second treatment, or the second treatment reduces symptoms to a greater extent, than would be seen if the second treatment were administered in the absence of the first treatment, or the analogous situation is seen with the first treatment. In certain embodiments, delivery is such that the reduction in a symptom, or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered.

In certain embodiments, a method or composition described herein is administered in combination with a nucleoside/nucleotide reverse transcriptase inhibitor (e.g., lamivudine, abacavir, zidovudine, stavudine, didanosine, emtricitabine, and tenofovir), a non-nucleoside reverse transcriptase inhibitor (e.g., delavirdine, efavirenz, etravirine, and nevirapine), a protease inhibitor (e.g., amprenavir, fosamprenavir, atazanavir, darunavir, indinavir, lopinavir, ritonavir, nelfinavir, saquinavir, and tipranavir), a fusion or entry inhibitor (e.g., enfuvirtide and maraviroc), integrase inhibitors (e.g., raltegravir and cabotegravir), or any combination thereof.

The present disclosure provides methods for promoting affinity maturation, and in particular in vivo affinity maturation, of antibodies. Further contemplated herein are methods for adoptively transferring an engineered B cell into a subject, such as a mammalian subject, to promote affinity maturation of the human antibody under mutation (or evaluation) in the subject (in the germinal center or lymph nodes). In various embodiments, the subject is a murine subject (e.g., a mouse). In some embodiments, the mammalian subject is a rodent. In some embodiments, the mammalian subject is a wild-type mouse. In some embodiments, the mammalian subject is not a transgenic mouse.

The details of one or more embodiments of the invention are set forth in the accompanying Figures, the Detailed Description, and the Examples. Other features, objects, and advantages of the invention will be apparent from the description and the claims.

### Evaluation of Antigens

The methods of the disclosure can also be used to evaluate and improve the design and immunogenicity of a desired antigen (e.g., an HIV-1 antigen). In some embodiments, only a properly assembled antigen is able to generate an immunogenic response (e.g., generate neutralizing antibodies). In some embodiments, the methods of the disclosure can be used to evaluate the immunogenicity of an antigen. In some embodiments, the methods of the disclosure can be used to evaluate the stability of an antigen. In some embodiments, the methods of the disclosure can be used to identify, purify, and/or perform quality control for a candidate antigen. In some embodiments, the antigen is a vaccine (e.g., an HIV-1 vaccine).

In some embodiments, the methods of the disclosure comprise introducing B cells modified to express the heavy chain variable region and/or a light chain variable region of a human antibody that specifically binds an antigen into a mammalian subject, and administering the antigen to the mammalian subject. In some embodiments, the methods of the disclosure comprise introducing B cells modified to express the heavy chain CDR3 of a human antibody that specifically binds an antigen into a mammalian subject, and administering the antigen to the mammalian subject. In some embodiments, the antigen may be administered more than once (e.g., 2, 3, 4, 5 or more times).

The edited B cells expand and affinity mature in vivo in response to the antigen. In some embodiments, the methods of the disclosure comprise determining affinity maturation of the engineered B cells. In some embodiments, the methods of the disclosure comprise determining proliferation of the engineered B cells. In some embodiments, the methods of the disclosure comprise determining class switching amongst the engineered B cells. In some embodiments, the methods of the disclosure comprise determining the neutralization response (e.g., generation of neutralizing antibodies).

### EXAMPLES

The invention is further illustrated by the following examples which are intended to illustrate but not limit the scope of the invention.

### Example 1. Introducing Exogenous Human Heavy- and Light-Chain Variable Segments into their Respective Native Loci

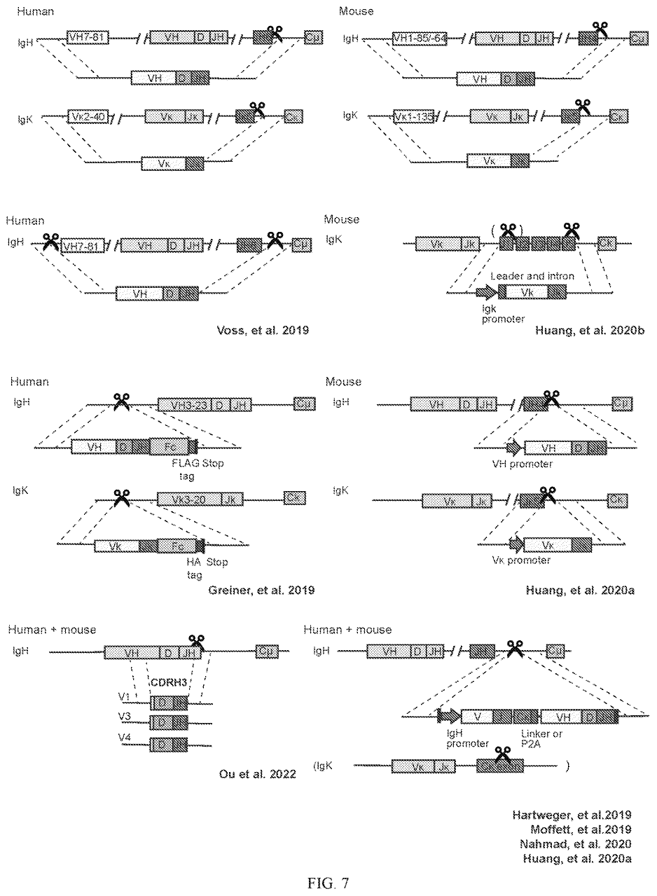

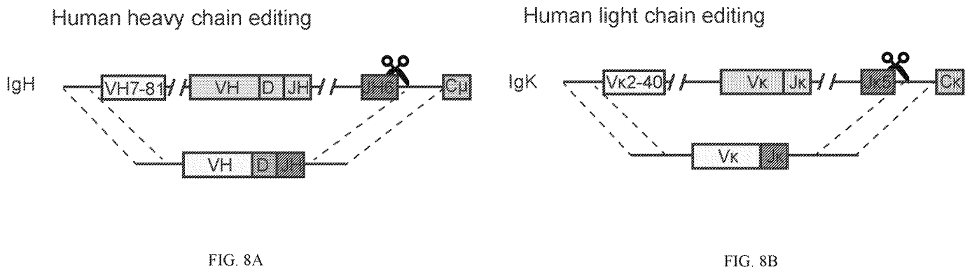

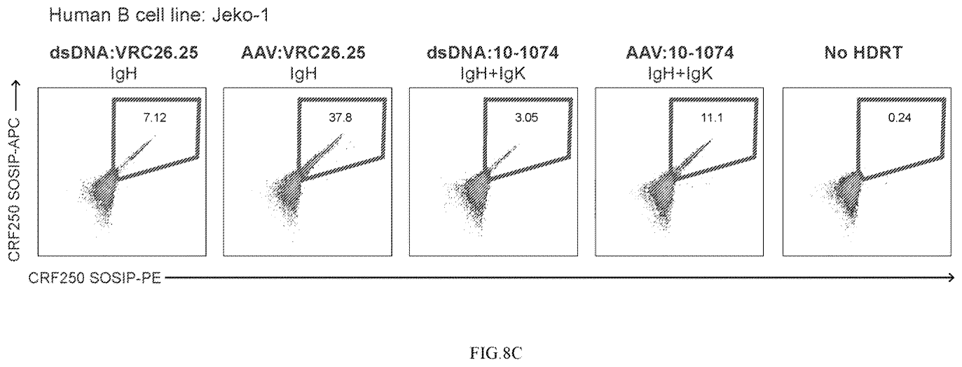

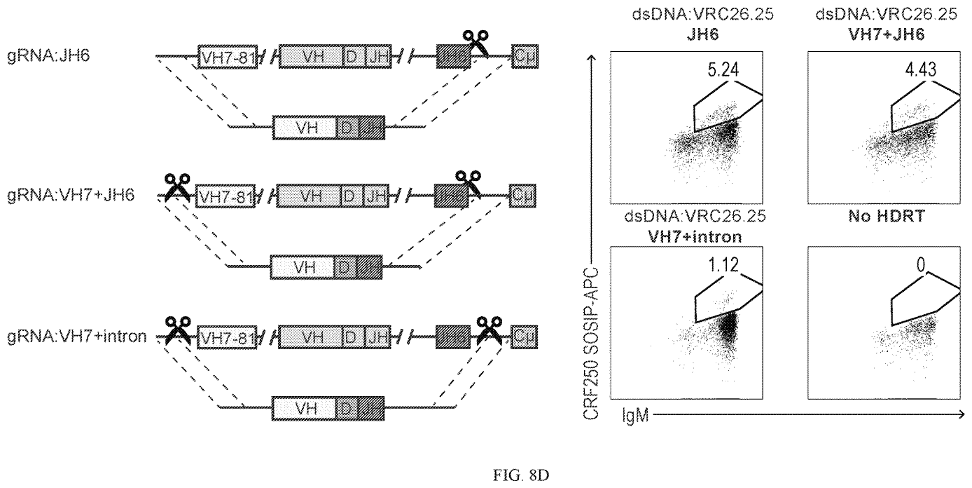

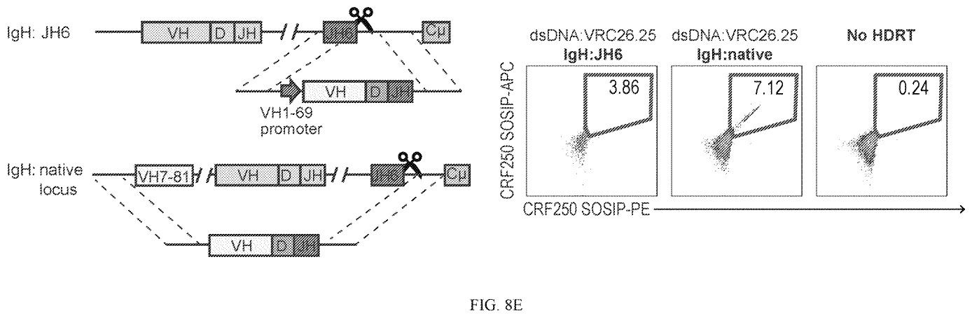

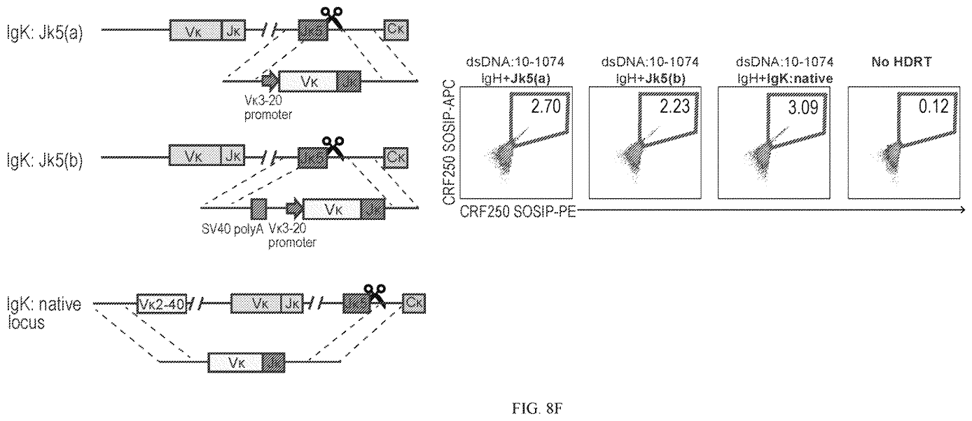

A general approach for directly replacing the heavy and light chains in human B cell was sought. First, it was determined whether homology-directed repair templates (HDRTs) with homology arms complementary to the promoter of the 5′-most heavy-chain variable (VH) segment (VH7-81 in humans), and immediately downstream of the 3′-most JH segment (JH6 in human), could template repair of a CRISPR/Cas 12a-mediated double-stranding break in the Jeko-1 B cell line, adapting an approach described in Voss et al. (15) (FIG. 7). VH7-81 and JH6 are separated by approximately 1 Mb in the unrecombined human chromosome and 108 kb in Jeko-1 cells. Jeko-1 cells were thus electroporated with MbCas12a ribonucleoproteins (RNPs) associated with a guide RNA (gRNA) targeting JH6 and an HDRT encoding the VRC26.25 heavy-chain variable gene bounded by the aforementioned homology arms (FIGS. 8A-8C). VRC26.25 was selected because its association with the HIV-1 envelope glycoprotein (Env) is almost wholly mediated by its heavy chain. It was observed that, using a dsDNA HDRT, 7% of cells could be edited to bind a soluble form of Env, namely a SOSIP. V7 based on the CRF250 HIV-1 isolate (CRF250-SOSIP) (16, 17). When adenoassociated virus 6 (AAV6) vector was used to deliver HDRTs, 38% of Jeko-1 cells could be edited, gRNA combinations were also compared, and a gRNA targeting JH6 alone was observed to have afforded the highest editing efficiency (FIG. 8D). An alternate strategy (11) for introducing an exogenous promoter and heavy-chain variable region, overwriting the final JH segment, was also less efficient than the native-loci approach (FIG. 8E). The possibility that this editing strategy could be combined with an analogous strategy targeting the kappa light-chain locus, using a HDRT complementary to the Vκ2-40 promoter and to a region immediately downstream of Jκ5, was further explored. Jeko-1 cells were simultaneously electroporated with RNPs associated with gRNAs targeting the JH6 and Jκ5 regions, respectively, and repair was directed with HDRTs with the aforementioned homology arms encoding the heavy and light chains of the bNAb 10-1074 (FIGS. 8A-8C). Unlike VRC26.25, 10-1074 requires both chains to bind Env (18). After editing with dsDNA or with AAV. 3% and 11% of cells, respectively, could bind SOSIP, indicating that heavy and light chains were successfully introduced at their native positions (FIG. 8C). Again, this approach afforded more efficient editing than one in which both heavy and kappa variable genes and an exogenous promoter were introduced in their respective introns (FIG. 8F).

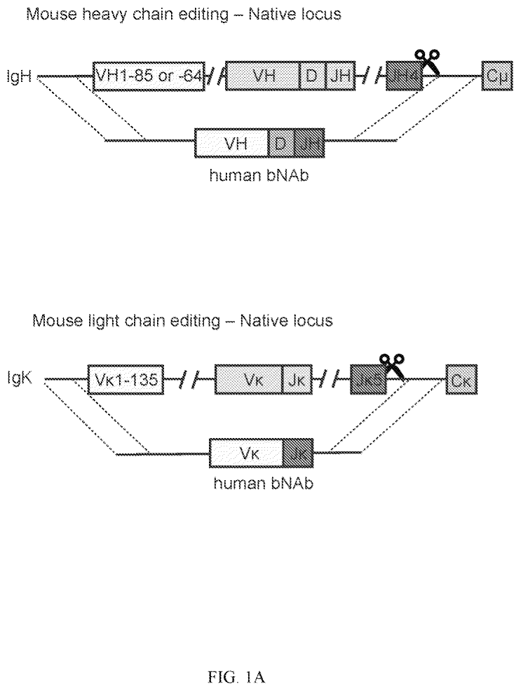

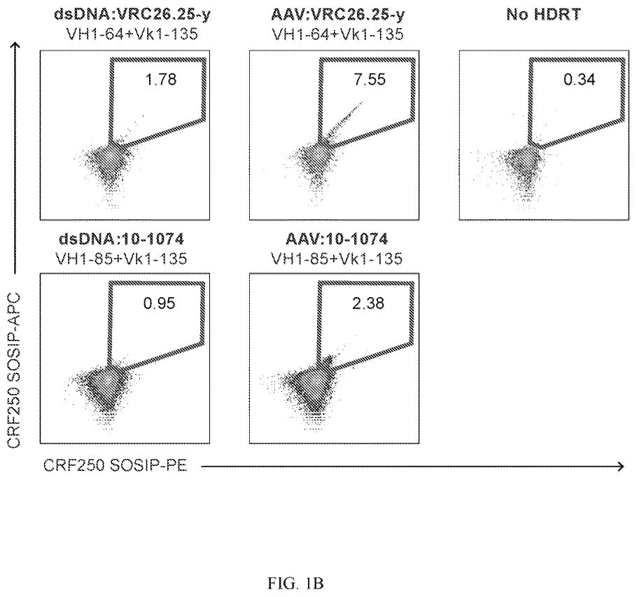

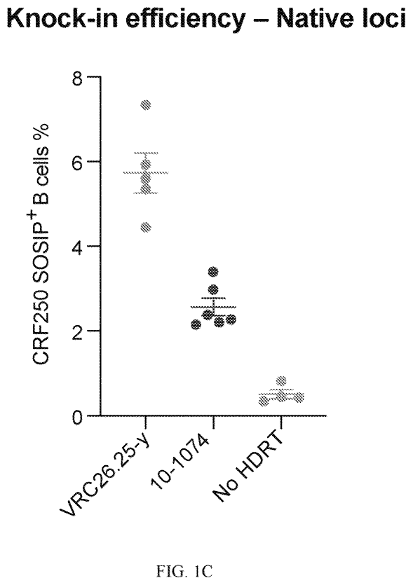

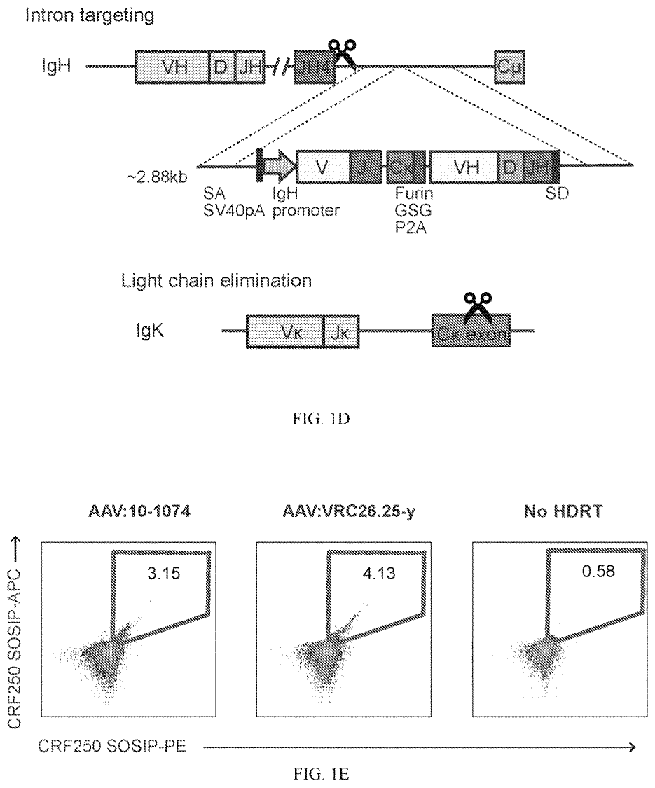

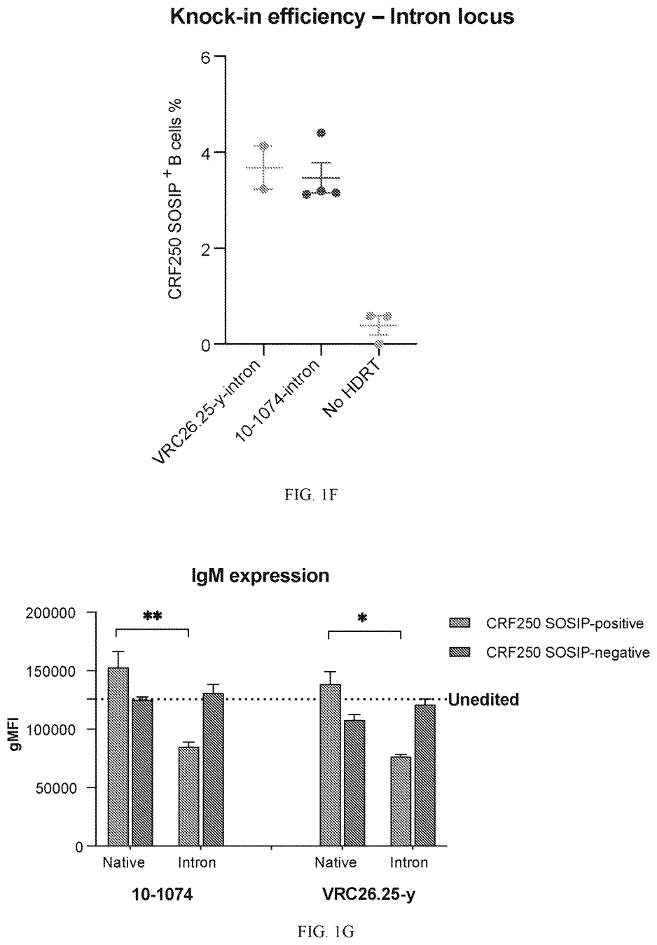

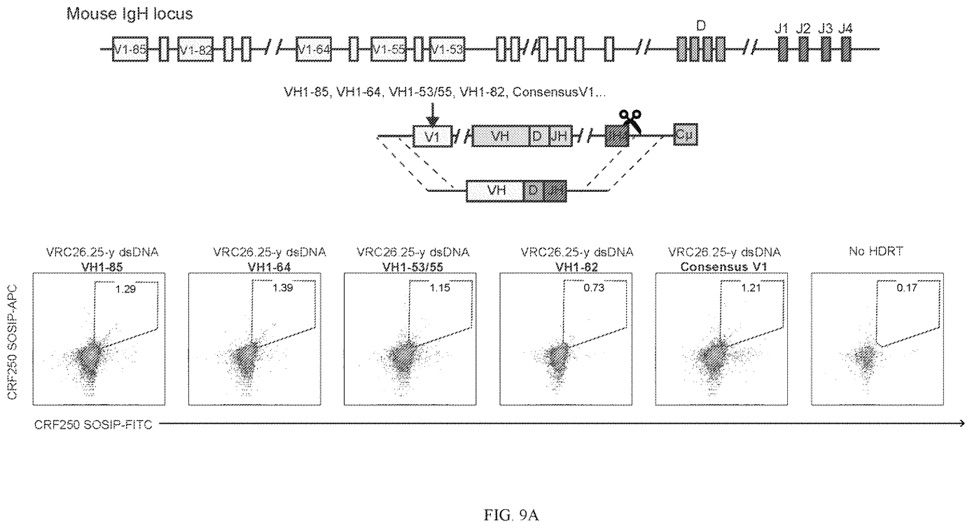

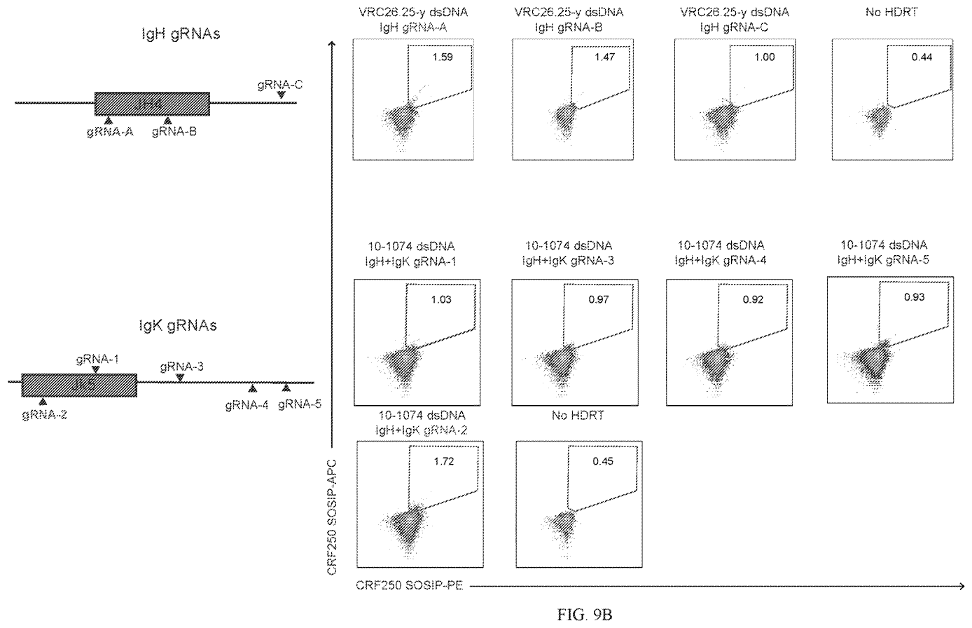

To determine if a similar strategy could be used to edit primary murine B cells, multiple 5′ HDRT homology arms and gRNAs were compared (FIGS. 9A-9B). The greatest editing efficiency was observed with HDRT complementary to the promoter regions of VH1-85 or VH1-64 (FIG. 1A). VH1-85 is the most 5′ of murine VH segments, while VH1-64 is a frequently utilized VH segment (19). Similarly, gRNA targeting the 3′-most JH segment, JH4, was used. Using the same principles, the kappa locus with HDRT complementary to the VK-135 promoter and downstream of Jκ5 was targeted. These HDRTs encoded, respectively, the heavy and light chains of either 10-1074 or VRC26.25-y, a previously described breadth- and potency-improved VRC26.25 variant (20). HDRTs were again co-electroporated as dsDNA with RNP or delivered by AAV-DJ. Markedly greater editing efficiencies were observed with AAV-delivered HDRT (FIG. 1B), approximately 6% and 3% for VRC26.25-y and 10-1074, respectively (FIG. 1C). Some antigen-positive B cells in the VRC26.25-edited population may express only the VRC26.25-y heavy chain. To compare this native-loci editing approach with the intron-based approach described in (8-12), a light-chain-P2A-heavy chain cassette described in Hartweger et al. (8), was introduced into the intron following JH4 and a lesion was also introduced in CK, limiting expression of the endogenous light chain (FIG. 1D). Editing efficiency in this case approached 4% (FIGS. 1E-1F). This intron-based approach results in significantly lower BCR expression than observed in native-loci edited cells (FIG. 1G). It was concluded that murine B cells could be edited in their native loci with efficiencies comparable to those of intron-based approaches, with higher BCR expression.

Native Loci-Edited Cells Generate More Potent Neutralizing Plasma after Immunization.

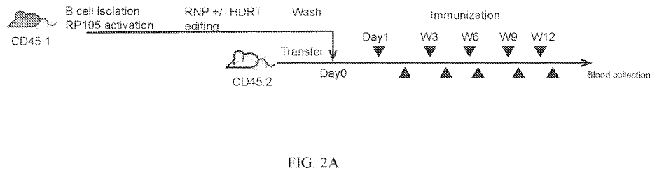

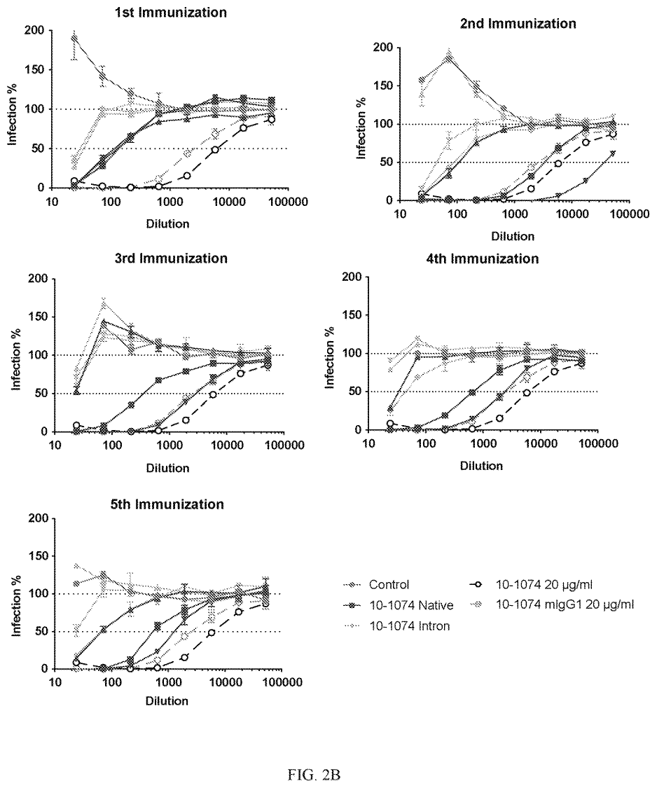

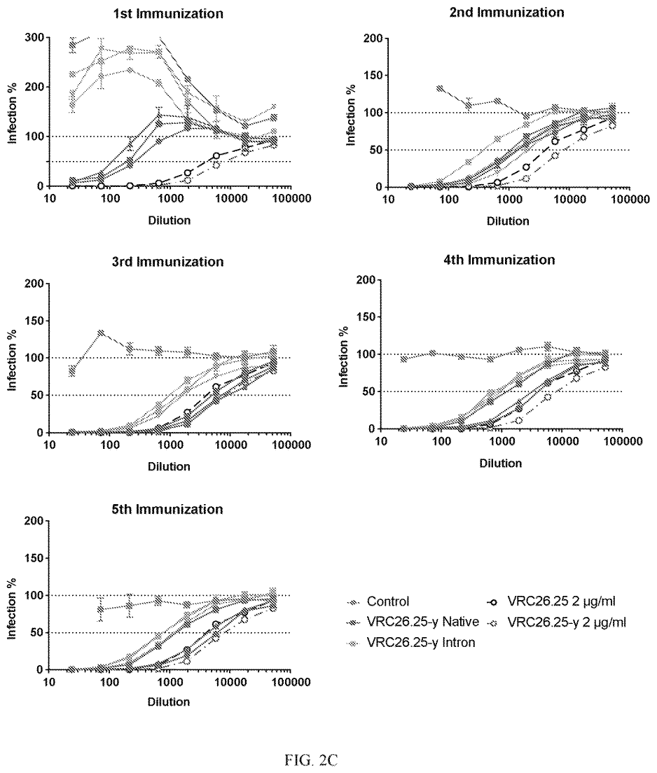

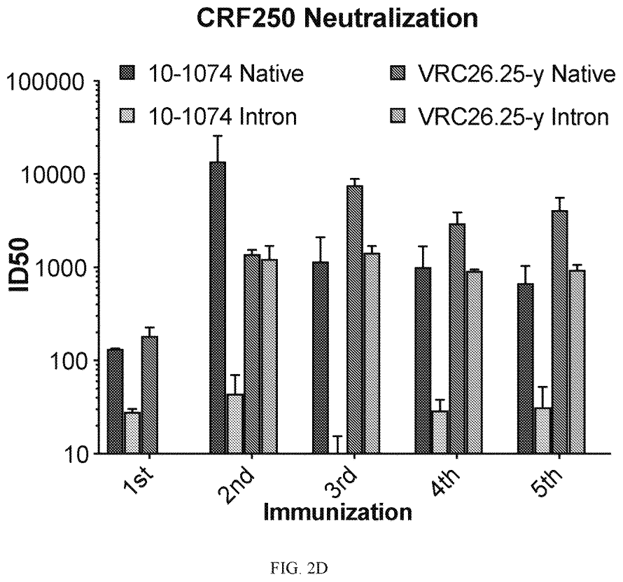

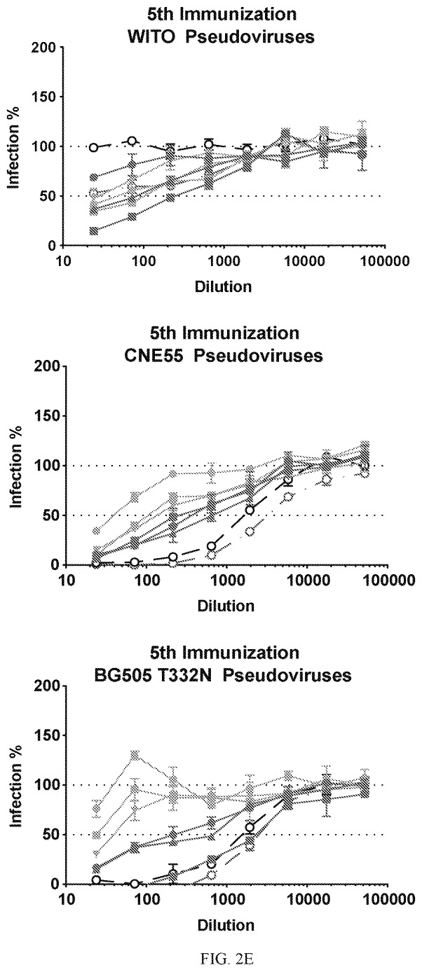

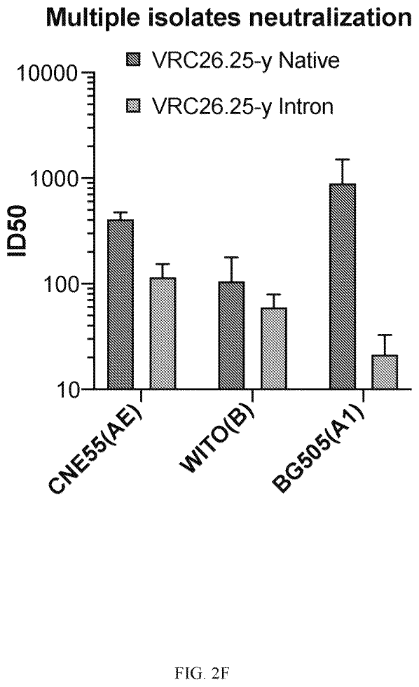

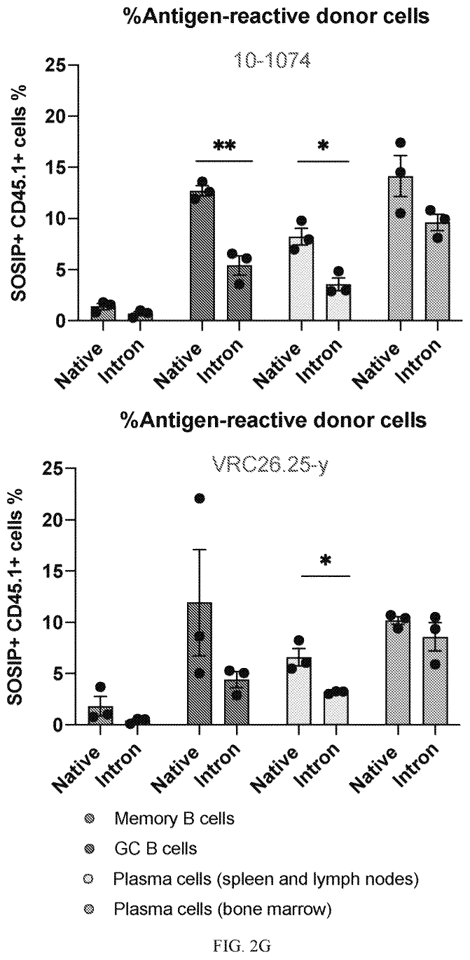

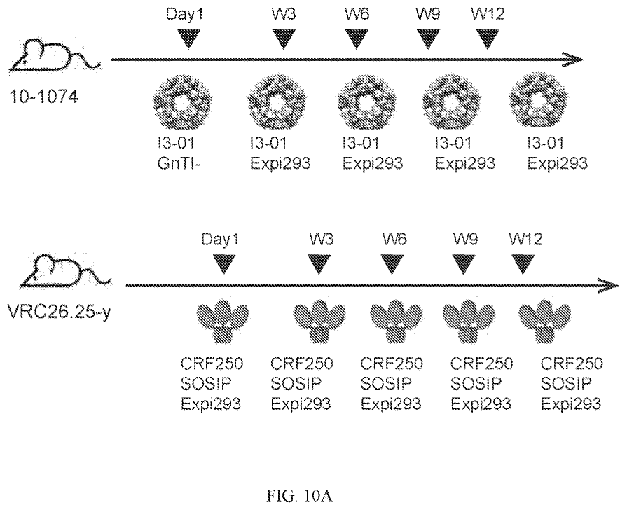

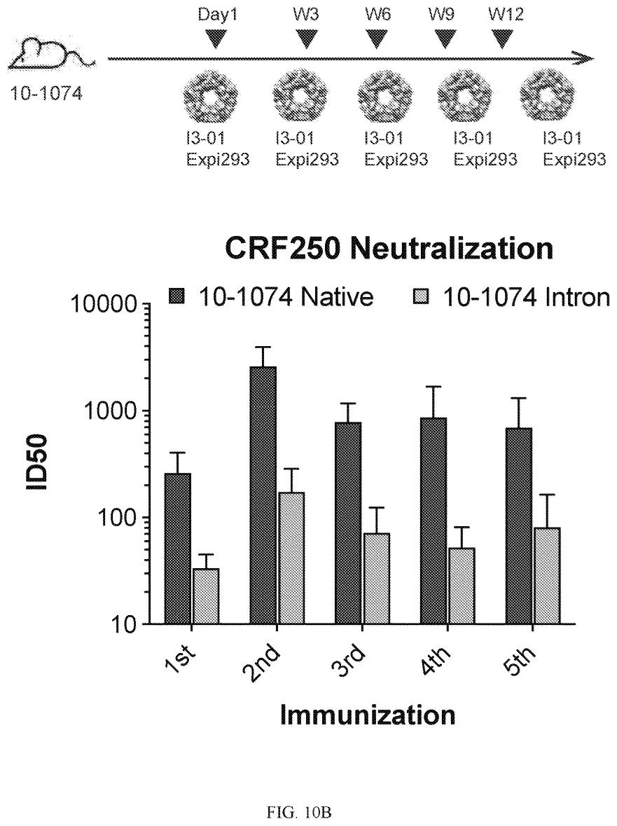

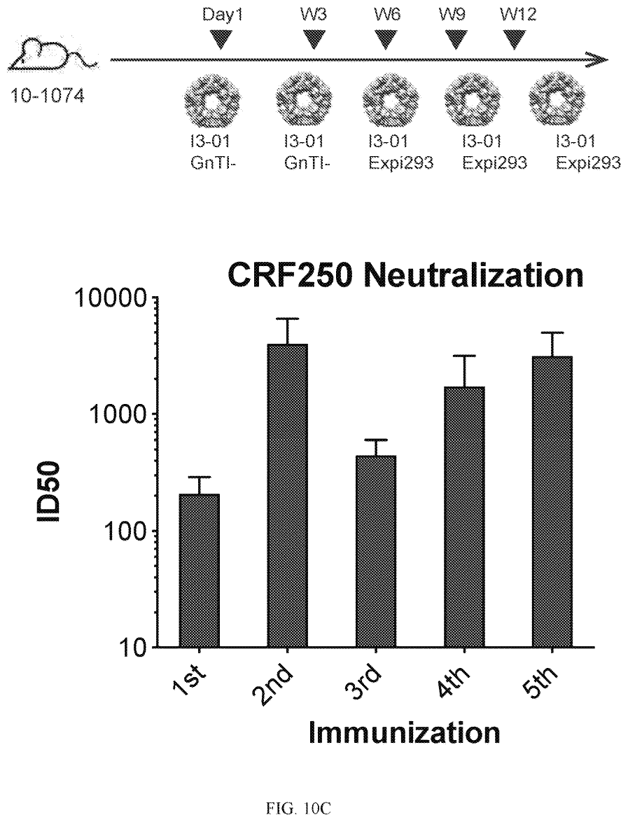

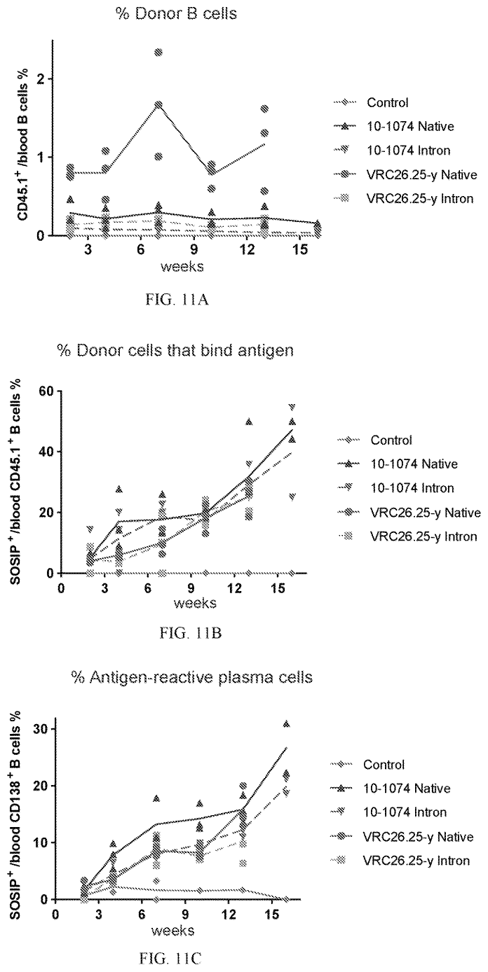

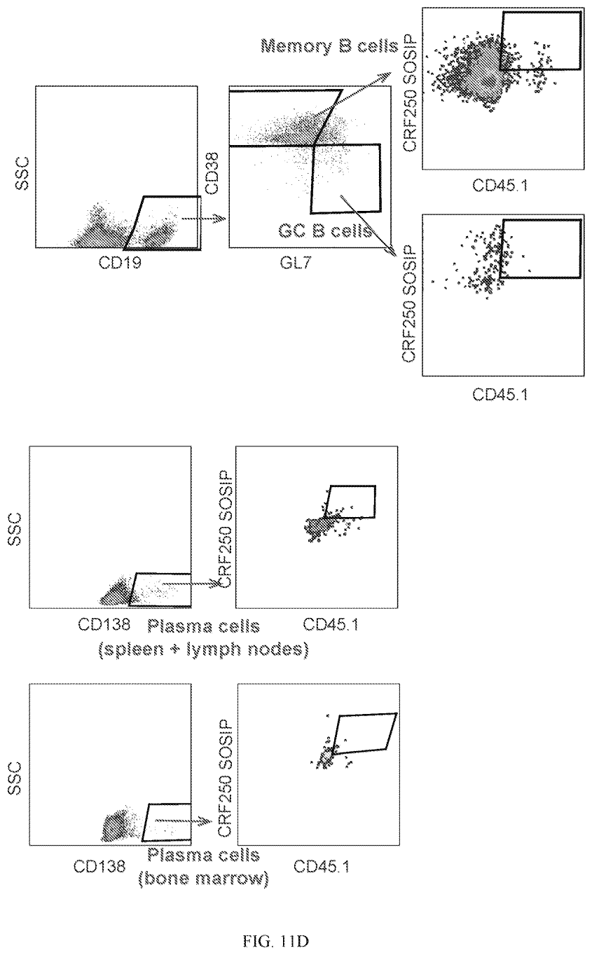

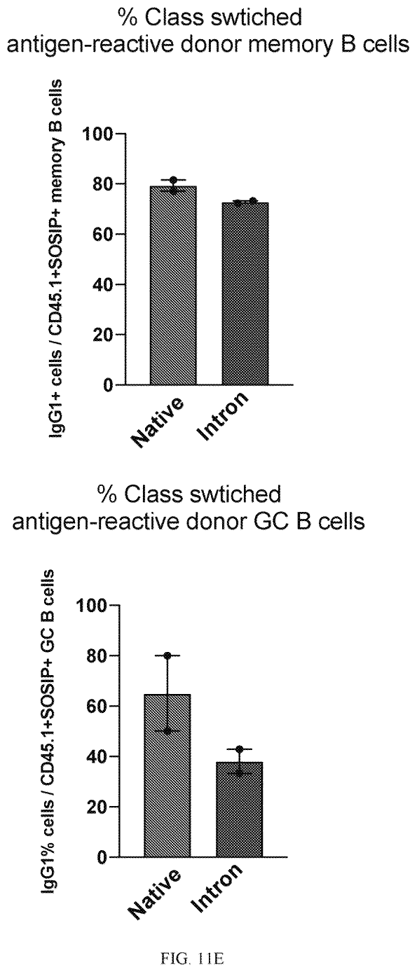

Next, it was sought whether differences in BCR expression or regulation would impact the development and maturation of B cells edited with native-loci or intron-based methods. B-cells isolated from spleens of CD45.1 donor mice were edited with each method and adoptively transferred to CD45.2 recipient mice (FIG. 2A). Mice were immunized one day after transfer and at weeks 3, 5, 9 and 12 thereafter, and blood was collected between immunizations. Mice receiving B cells engineered to express 10-1074 were immunized with a 60-mer 13-01 scaffold presenting the BG505 gp120 subunit of Env (21, 22) (FIG. 10A). Mice receiving VRC26.25-y edited B cells were immunized with CRF250-SOSIP. Blood plasma samples were characterized for their ability to neutralize CRF250 pseudoviruses. Importantly, plasma from mice that did not receive engineered B cells but which were otherwise vaccinated on the same schedule did not neutralize CRF250 pseudoviruses (FIGS. 2B-2C). The potency of response was consistently greater with native-loci editing for both antibodies after each of five immunizations (FIGS. 2B-2D), and differences were more dramatic after earlier immunizations. These differences extended to neutralization with isolates divergent from CRF250, namely WITO, CNE55, and BG505-T332N (FIGS. 2E-2F). Of note. VRC26.25-y plasma neutralized the VRC26.25-resistant WITO isolate, consistent with the greater breadth of the VRC26.25-y variant (20). Two additional studies of 10-1074 showed that neutralization potency against CRF250 pseudoviruses consistently reached a peak after the second vaccination and remained steady after subsequent vaccinations (FIGS. 10B-10C). Additionally, the persistence and development of antigen-binding donor cells was monitored by flow cytometry. CD45.1+ donor cells comprised 0.2-2% of B cells in the blood, remaining relatively stable after the first immunization in all groups (FIG. 11A). However, the percent of donor cells that bound antigen steadily increased after vaccination in all four groups, rising from 5% to 40% in most cases (FIG. 11B). The fraction of donor and recipient plasma cells that bound antigen similarly rose to 10-20% in all four groups (FIG. 11C). After the final vaccination, the fraction of donor cells that were antigen-positive in four populations were determined to be: memory B cells, germinal center B cells, plasma cells isolated from spleen and lymph nodes, and bone marrow plasma cells (FIG. 11D). Native-loci edited cells showed higher fractions of antigen-binding donor cells in all four B-cell populations, with significant differences with 10-1074-edited cells in the number of germinal center B cells and spleen- and lymph node-derived plasma cells (FIG. 2G). Slightly higher frequency of antigen-positive donor cells that class-switched to the IgG1 isotype in the memory and GC populations in the native-loci-edited cells were also observed (FIG. 11E). It was concluded that mice engrafted with native-loci edited B cells expand more efficiently and generate more potent neutralizing plasma than intron-edited cells.

### Example 2. VRC26.25-y and 10-1074-Expressing B Cells can be Combined to Provide Broader Protection

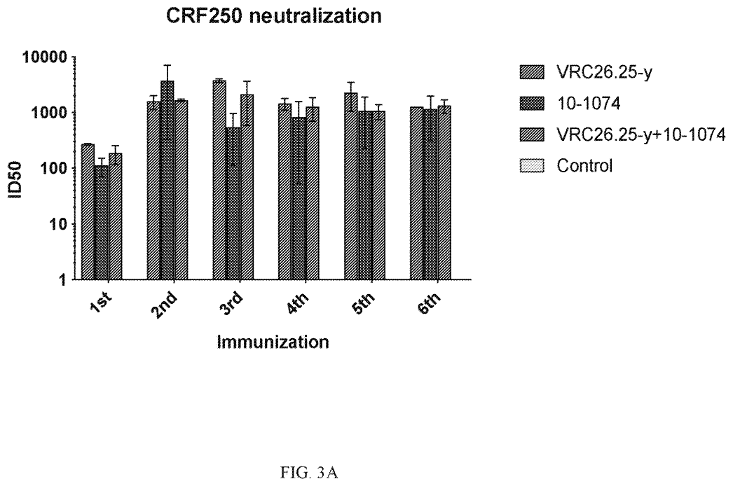

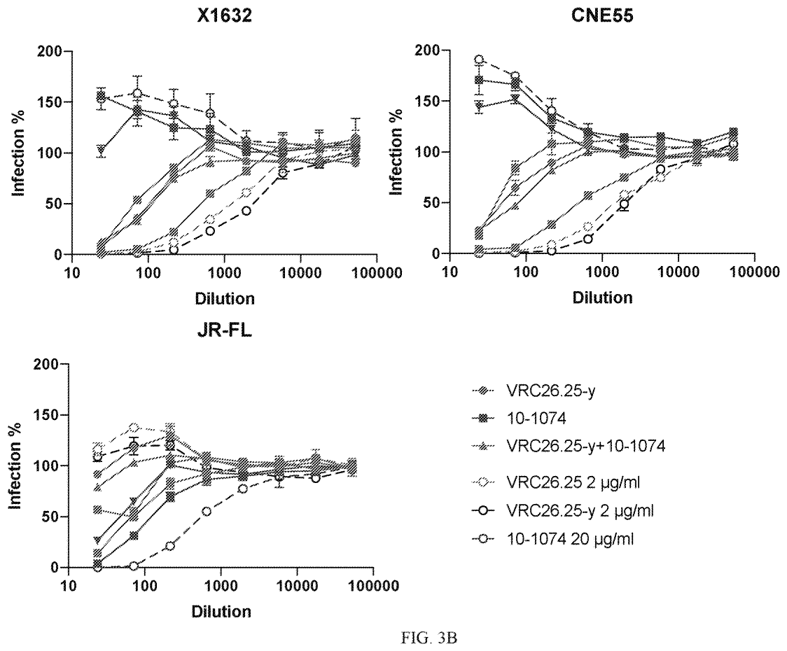

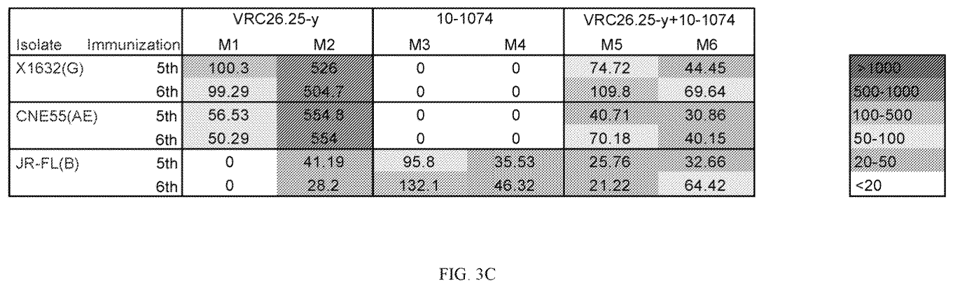

The impact of combining B cells edited to express two different classes of bNAbs on the breadth and potency of the neutralizing plasma was explored. A 50:50 mixture of 10-1074- and VRC26.25-y-edited cells was adoptively transferred to recipient mice. These mice were compared to mice receiving the same total number of edited cells expressing either 10-1074 or VRC26.25-y alone. All mice were vaccinated with CRF250-SOSIP using a schedule similar to that shown in FIG. 2A, except that a 6th vaccination was included. Plasma from mice receiving the cells engineered to express both bNAbs neutralized CRF250 pseudoviruses with efficiencies similar to those transferred with either bNAb alone (FIG. 3A), consistent with the sensitivity of CRF250 to both bNAbs. As anticipated, plasma from mice receiving only 10-1074-edited cells failed to neutralize pseudoviruses of 10-1074-resistant isolates X1632 and CNE55, and plasma from those receiving only VRC26.25-y-edited cells poorly neutralized the VRC26.25- and VRC26.25-y-resistant JRFL isolate (FIGS. 3B-3C). Notably, one of the latter mice (M2) gained the ability to neutralize JRFL. In contrast to these mice, mice receiving a mixture of engineered B cells neutralized all three isolates, indicating that both bNAbs expressed and were secreted. Moreover, average neutralization (ID₅₀) against all three isolates increased with vaccination of these mice (FIG. 3C), indicating that CRF250-SOSIP promoted expression of both bNAbs. It was concluded that B cells engineered to express different bNAbs can be combined to increase the breadth of response to HIV-1.

### Efficient Somatic Hypermutation of Native-Loci Edited B Cells.

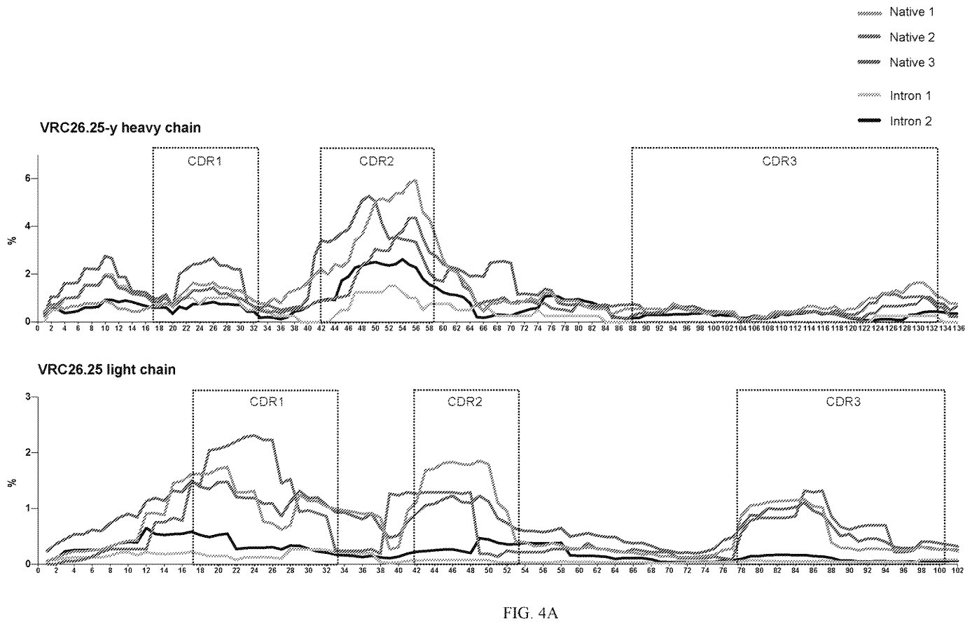

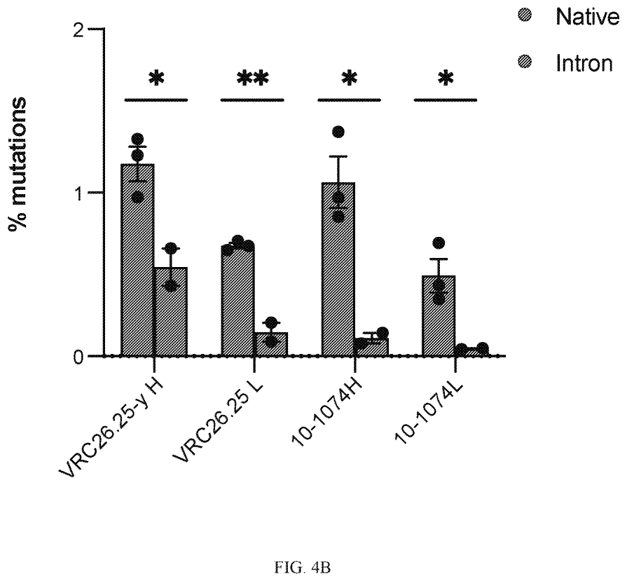

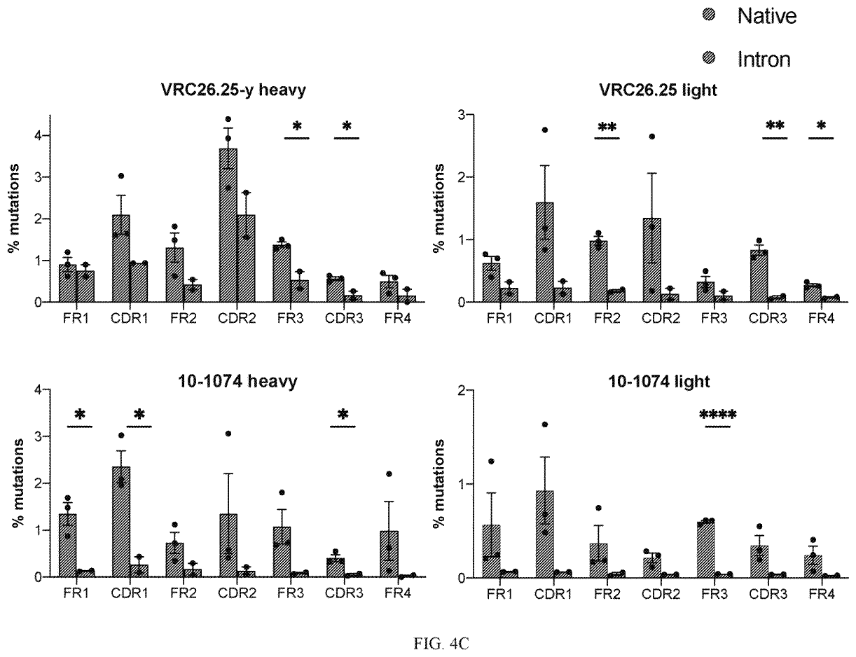

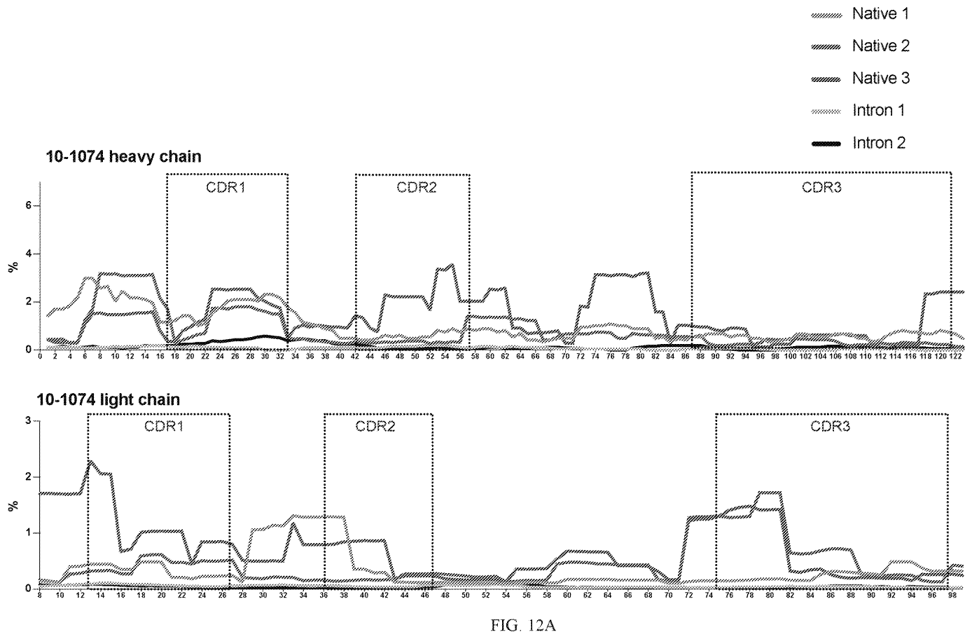

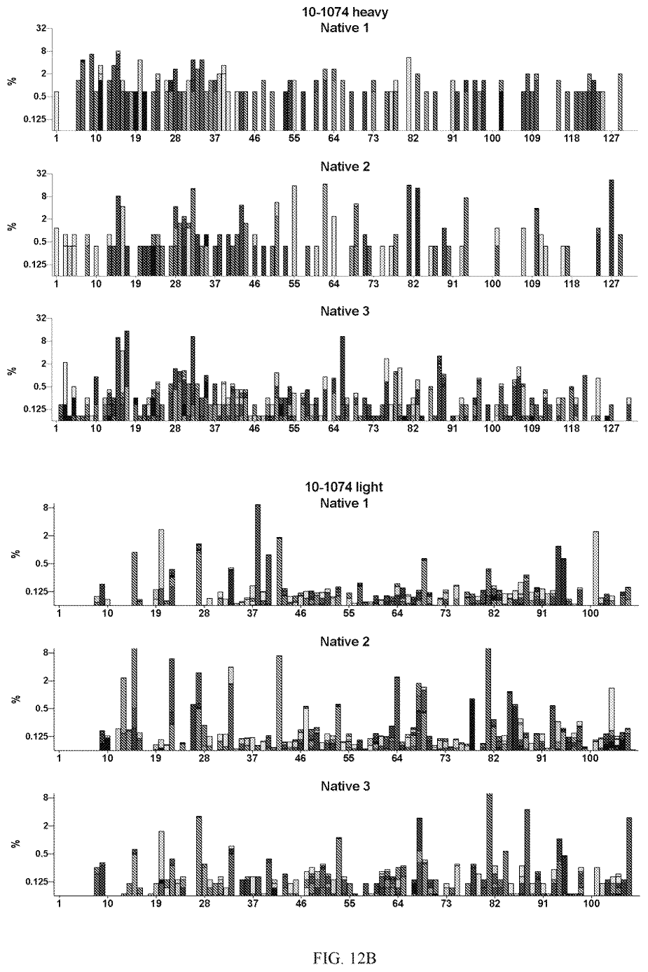

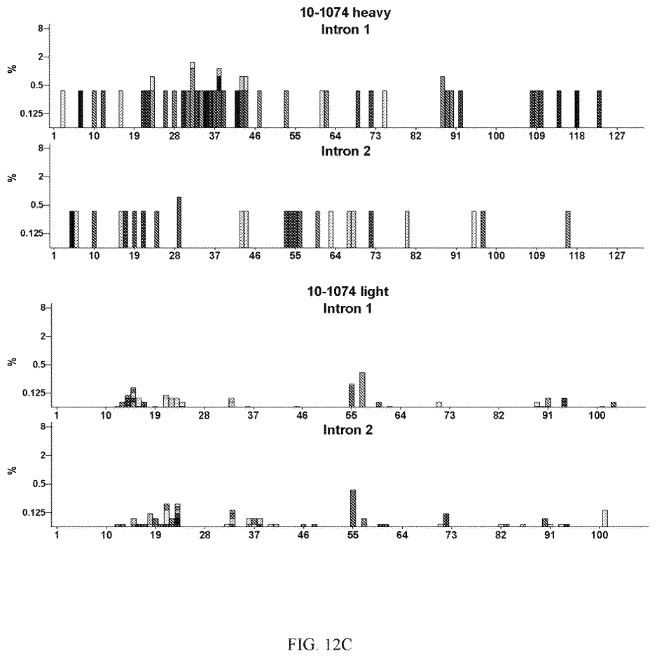

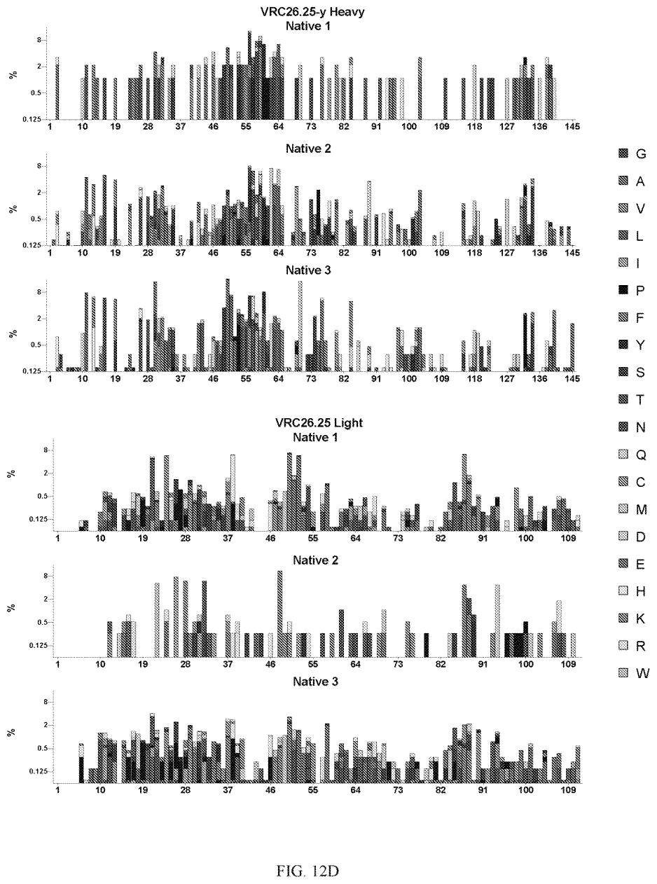

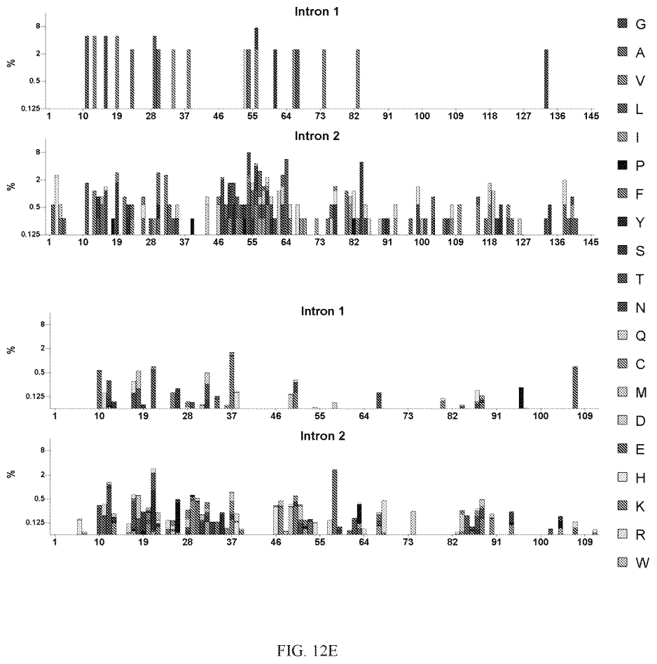

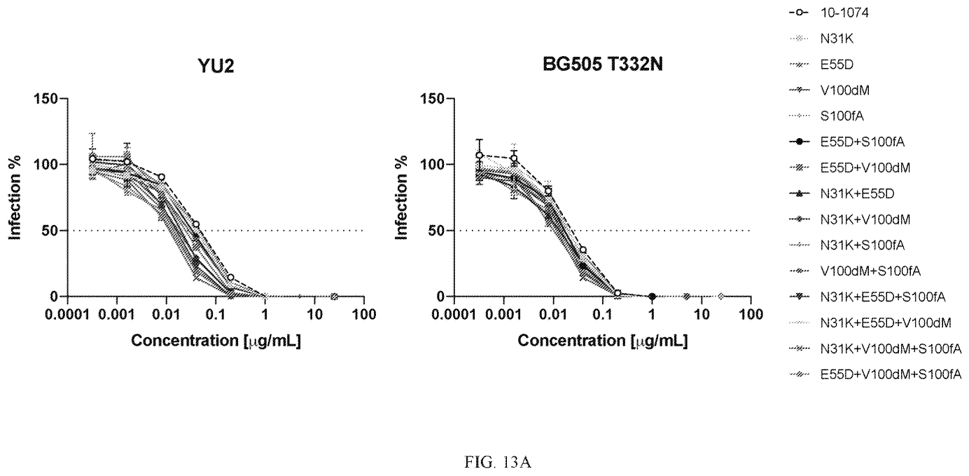

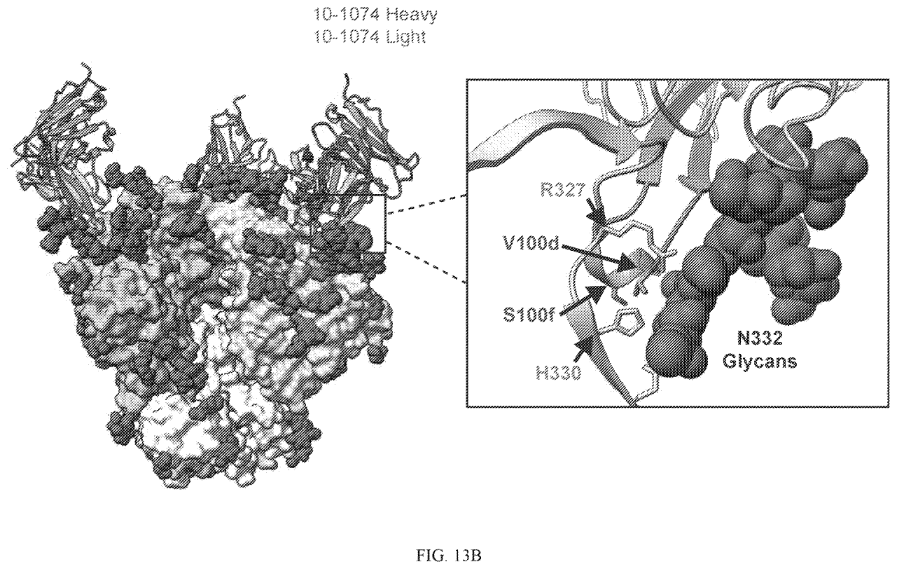

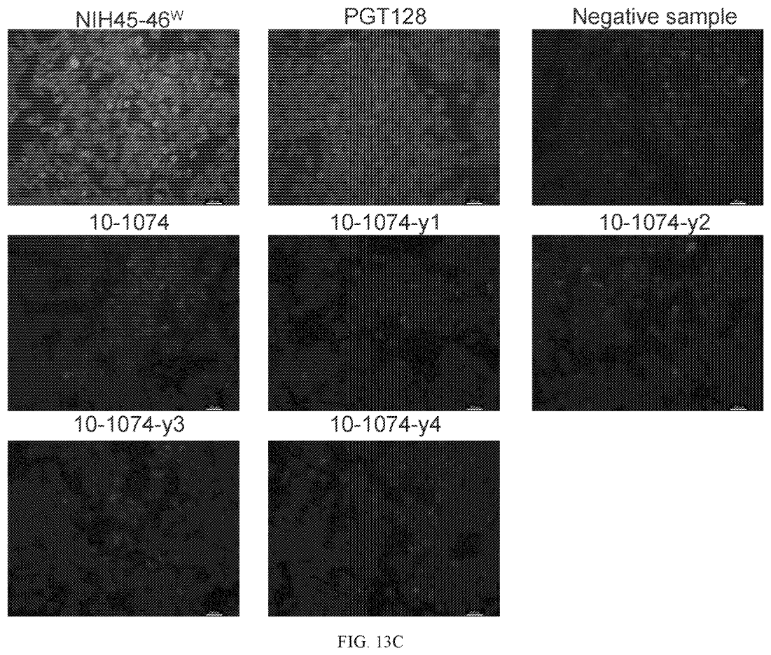

One week after the final immunization, cells were harvested from the lymph nodes and spleen of mice whose plasma was characterized in FIGS. 2A-2G. Successfully edited CD45.1-positive donor cells were analyzed by next-generation sequencing (NGS) to determine their rate of somatic hypermutation (SHM). The average frequency of SHM, based on a sliding window of 10 residues, is depicted for each mouse edited to express VRC26.25-y (FIG. 4A) or 10-1074 (FIG. 12A). The pattern of hypermutation is different in the codon-optimized 10-1074 than it is from the donor-derived VRC26.25-y sequence, perhaps reflecting a different distribution of mutational hotspots. Alternatively, different selection pressures on the two bNAbs could alter hypermutation patterns. However, in both cases, the rate of SHM of both heavy- and light-chains was significantly greater in native-edited cells than in intron-edited cells (FIG. 4B; FIGS. 12B-12E). These differences were more pronounced in the CDR regions of VRC26.25-y and the heavy-chain CDR regions of 10-1074 than in their corresponding framework regions (FIG. 4C). It was concluded that, compared with intron-targeted editing, native-loci editing results in markedly greater SHM rates, and the distribution of mutations is more CDR-focused.

Affinity Maturation of a Human bNAb in Mice.

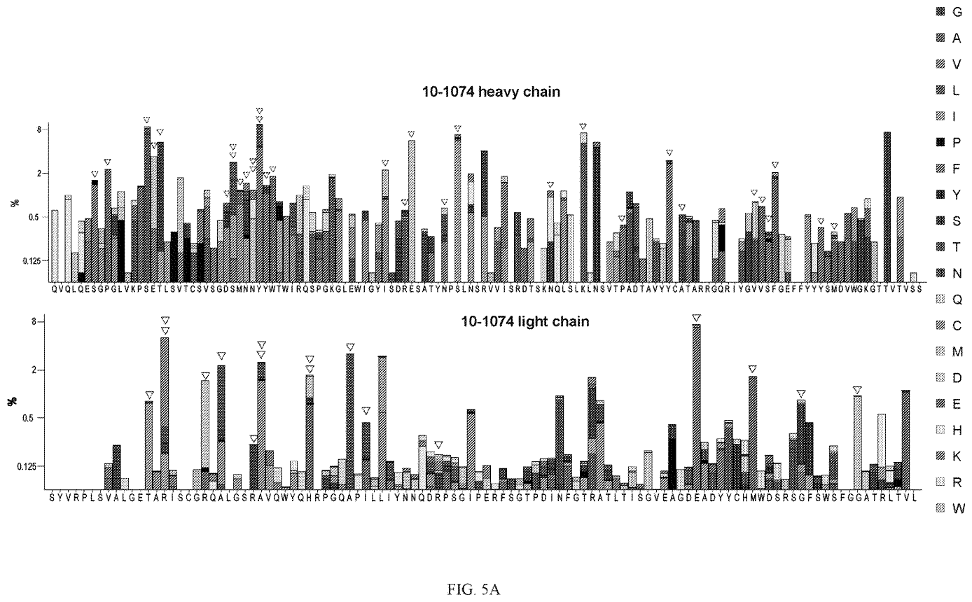

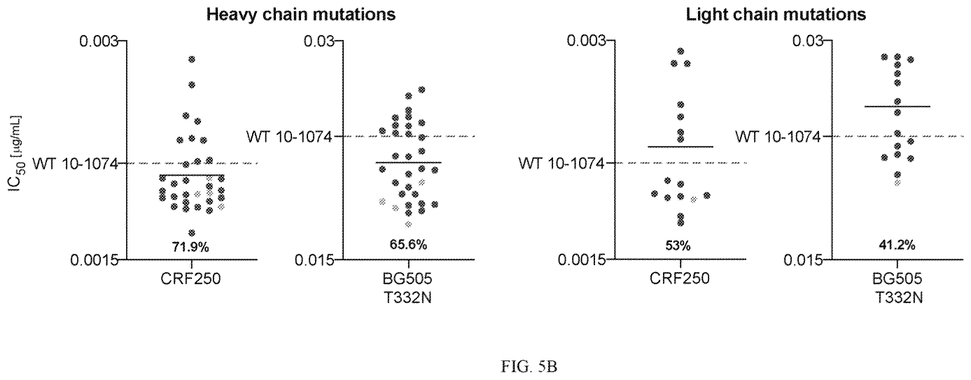

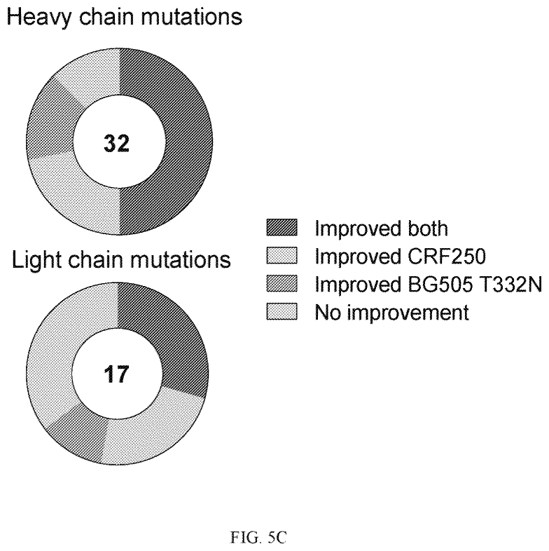

Whether the mutations observed in edited BCRs improved the potency of these bNAbs was sought. The focus was on 10-1074 mutations because the potency of this bNAb is lower than that of VRC26.25-y, affording greater room for improvement. High-frequency mutations that emerged in at least two native-edited 10-1074 mice were further analyzed (FIG. 5A. FIG. 12B). With these 35 criteria, 32 heavy-chain mutations and 17 light-chain mutations found in 10-1074-edited B cells were identified (indicated with arrowheads in FIG. 5A). Each of these mutations was introduced individually into 10-1074 itself and characterized for its ability to neutralize either CRF250 or BG505-T332N isolates. The majority of heavy- and light-chain mutations improved neutralization against at least one of these isolates, and the geometric mean IC₅₀ of heavy-chain 10-1074 variants was lower than the IC₅₀ of unmodified 10-1074 (FIG. 5B). Half (16/32) of heavy-chain mutations and 29% (5/17) of light-chain mutations improved neutralization against both isolates (FIG. 5C). Interestingly, three of these heavy-chain mutations (N31D, S100fA, and Y100 nF), were also found in the antibody PGT121, a V3-glycan antibody similar to 10-1074 that emerged from the same progenitor B cell in the same donor (18). Collectively, these results demonstrated that the 10-1074 BCRs expressed in native-loci-edited murine B cells affinity matured in response to antigen.

More Potent 10-1074 Variants with Low Polyreactivity.

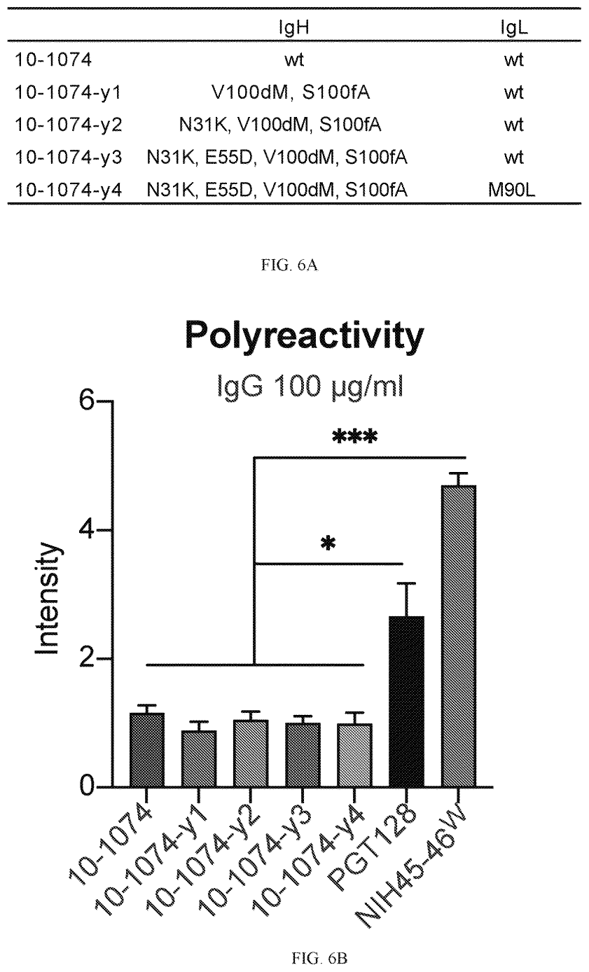

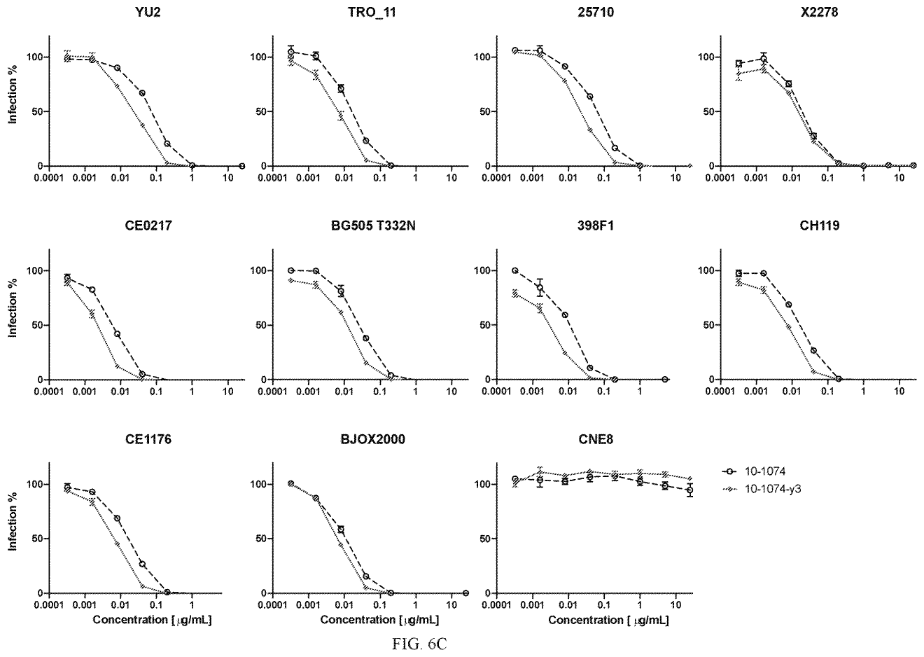

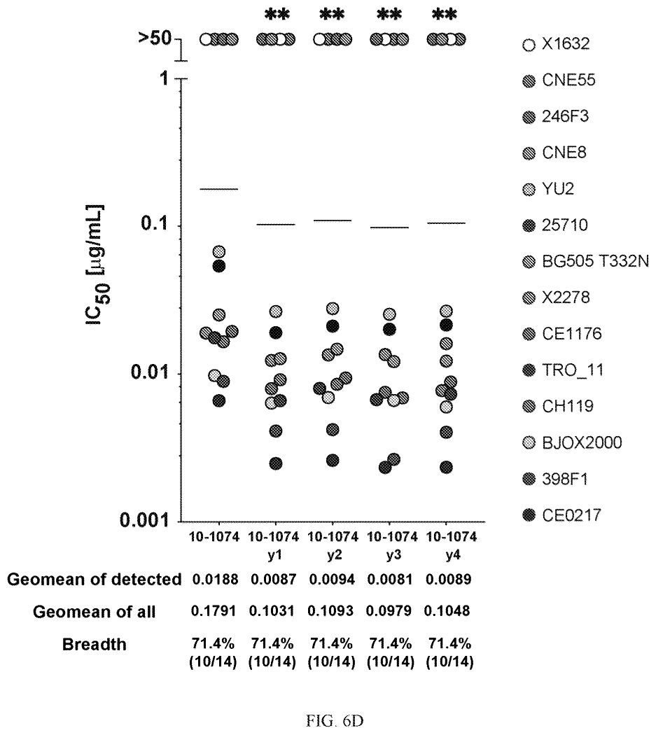

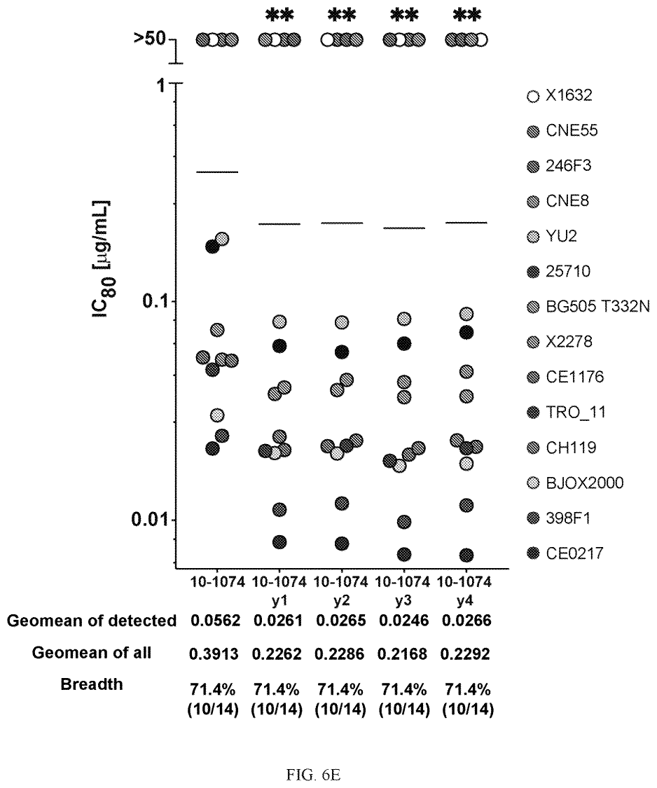

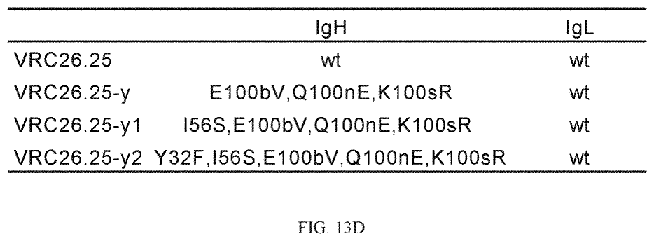

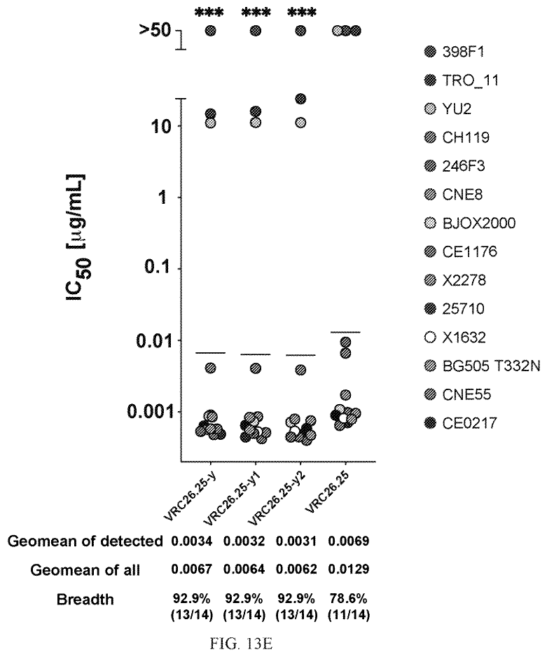

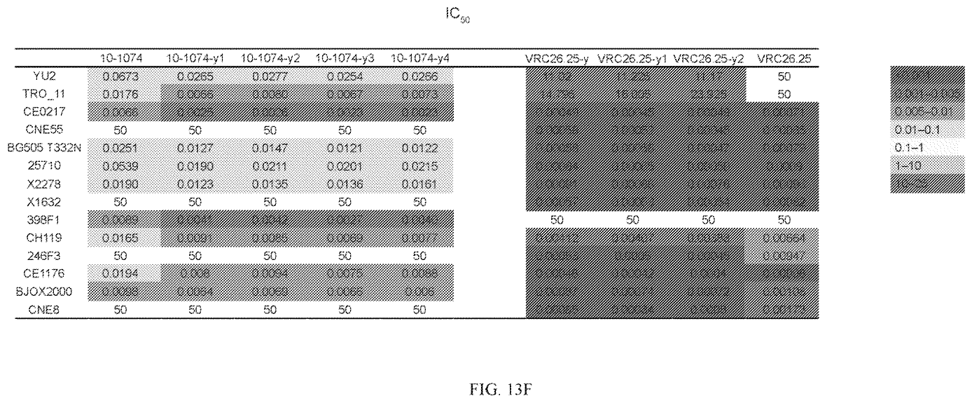

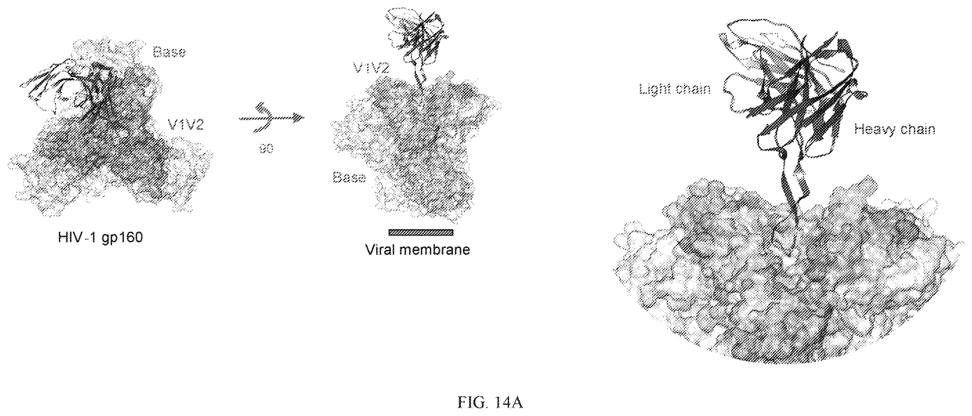

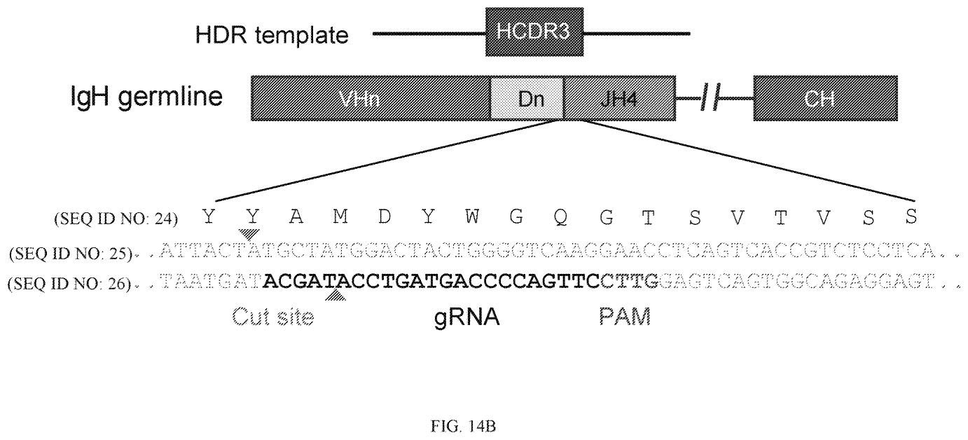

Five 10-1074 mutations that most effectively enhanced neutralization of both CRF250 and BG505-T332N isolates were identified, combined in various ways, and characterized by their ability to neutralize two selected isolates, YU2 and BG505-T332N (FIG. 13A). YU2 was used to originally identify 10-1074 (18), and BG505-T332N was used to immunized 10-1074 mice here. It was observed that 10-1074 variants with both V100dM and S100fA consistently neutralized these isolates more efficiently that wild-type 10-1074. These mutations were localized at the heavy-chain CDR3. V100d directly contacts a proteinaceous region in the third variable loop of Env as well as the glycan at asparagine 332. S100f also directly contacted this glycan (FIG. 13B). Four new 10-1074 variants were generated accordingly, 10-1074-y 1 was denoted through-y4, and each included these V100dM and S100fA mutations (FIG. 6A). All four variants retained the low polyreactivity of 10-1074 itself when compared with the polyreactive bNAbs PGT128 and NIH45-46W (23-25) (FIG. 6B and FIG. 13C). Every variant also neutralized all 10-1074-sensitive members of global panel of HIV-1 isolates more efficiently than 10-1074 itself, with significantly lower geometric mean IC₅₀ and IC₈₀ values (FIGS. 6C-6E). Among these variants, 10-1074-y3 was slightly more potent on average. These mutations did not broaden 10-1074 as none of the four 10-1074-resistant variants in the global panel were neutralized. A more modest effort to improve VRC26.25-y did not result in further increases in potency, perhaps reflecting the very high potency of VRC26.25-y itself (FIGS. 13D-13F). However, an I56S mutation—a reversion to the germline V3-30 sequence—maintained or improved the potency of VRC26.25-y against most isolates, suggesting that some bNAb hypermutations can be reverted to their corresponding germline residue without loss of activity. Thus. 10-1074-y3 is equally polyreactive and significantly more potent than wild-type 10-1074. It was concluded that affinity maturation of native-loci-edited B cells in mice can facilitate development of more potent antibodies.

### DISCUSSION

Markedly higher neutralization activity in plasma of mice engrafted with native loci-edited B cells was observed, especially following earlier immunizations, compared to a published intron-targeting approach (8). Significantly greater rates of somatic hypermutation were observed in these cells. These high SHM rates facilitated affinity maturation of BCRs engineered to express the variable chains of the bNAb 10-1074. Robust SHM and further maturation of edited BCRs suggest that native-loci editing does not significantly disrupt development or function of the edited B cell. In contrast, lower SHM rates were observed with an intron-targeting approach. These rates were consistent with previous reports (11, 14) and suggested that elements of the intron-targeting cassette interfere with processes necessary for a potent and diverse antibody response.

In addition to high hypermutation rates, a high frequency of potency-enhancing mutations emerging from native loci-edited cells was observed, especially in the 10-1074 heavy-chain. This observation suggested that animal immune systems might be employed to improve the potency and bioavailability of antibodies or biologics. The mammalian germinal center may more efficiently generate and select high-affinity antibodies than in vitro methods like phage-, yeast-, and mammalian cell-display techniques. The continuous diversification and coordinated selection process in germinal centers may more effectively explore useful sequence space over time. It was hypothesized that mammalian immune systems select against BCRs that aggregate, recognize self, or are easily proteolyzed in vivo. If so, in vivo affinity maturation would co-select for antibodies with higher bioavailability, lower affinity to self antigens, and longer functional half-lives. This hypothesis is consistent with the low polyreactivity of the four potency-enhanced 10-1074 variants of FIGS. 6A-6E.

Accordingly, provided in the present disclosure are affinity-matured variants of the 10-1074 antibody generated by the directed in vivo evolution and maturation methods disclosed herein. In some embodiments, these variants comprise an amino acid sequence having at least 85%, 90%, 92.5%, 95%, 98%, or 99% identity to any of SEQ ID NOs: 16-21. In some embodiments, the variant comprises the amino acid sequence of any of SEQ ID NOs: 16-21. In some embodiments, the variant comprises the amino acid sequence of any of SEQ ID NOs: 16 and 18-21. In exemplary embodiments, the variant comprises the amino acid sequence of SEQ ID NO: 16, SEQ ID NO: 18. SEQ ID NO: 19, SEQ ID NO: 20, or SEQ ID NO: 21. The disclosed variants may comprise an amino acid sequence that differs by 1, 2, 3, 4, 5, 6, 7, 8, 9, 10, 10-15, 15-20, or more than 20 amino acids from any of SEQ ID NOs: 16-21. The disclosed variants may comprise an amino acid sequence that contains about 3, 5, 7, 10, 15, 20, 25, 30, 35, or more than 35 consecutive amino acids in common with any of SEQ ID NOs: 16-21.

The sequences of the wild-type 10-1074 heavy and light chains, and variants thereof, are shown below:

Wild-type 10-1074 heavy chain,
SEQ ID NO: 1
QVQLQESGPGLVKPSETLSVTCSVSGDSMNNYYWTWIRQSPGKGLEWIG
YISDRESATYNPSLNSRVVISRDTSKNQLSLKLNSVTPADTAVYYCATA
RRGQRIYGVVSFGEFFYYYSMDVWGKGTTVTVSSASTKGPSVFPLAPSS
KSTSGGTAALGCLVKDYFPEPVTVSWNSGALTSGVHTFPAVLQSSGLYS
LSSVVTVPSSSLGTQTYICNVNHKPSNTKVDKRVEPKSCDK
Wild-type 10-1074 light chain,
SEQ ID NO: 2
SYVRPLSVALGETARISCGRQALGSRAVQWYQHRPGQAPILLIYNNQDR
PSGIPERFSGTPDINFGTRATLTISGVEAGDEADYYCHMWDSRSGFSWS
FGGATRLTVLGQPKAAPSVTLFPPSSEELQANKATLVCLISDFYPGAVT
VAWKADSSPVKAGVETTTPSKQSNNKYAASSYLSLTPEQWKSHRSYSCQ
VTHEGSTVEKTVAPTECS

The four variants of 10-1074 are disclosed in FIG. 6 with the mutations in heavy chains and light chains:
10-1074-y1: Heavy chain, V100dM; S100LA (Capital indicates original and mutated amino-acid; number and lowercase indicates residue position);
10-1074-y2: Heavy chain, N31K; V100dM; S100fA;
10-1074-y3: Heavy chain, N31K; E55D; V100dM; S100fA;
10-1074-y4: Heavy chain, N31K; E55D; V100dM; S100fA; Light chain: M90L.

10-1074-y1: Heavy chain, V100dM; S100fA
(SEQ ID NO: 16)
QVQLQESGPGLVKPSETLSVTCSVSGDSMNNYYWTWIRQSPGKGLEWIG
YISDRESATYNPSLNSRVVISRDTSKNQLSLKLNSVTPADTAVYYCATA
RRGQRIYGMVAFGEFFYYYSMDVWGKGTTVTVSSASTKGPSVFPLAPSS
KSTSGGTAALGCLVKDYFPEPVTVSWNSGALTSGVHTFPAVLQSSGLYS
LSSVVTVPSSSLGTQTYICNVNHKPSNTKVDKRVEPKSCDK
(mutations bolded and underlined)
10-1074-y1 light chain
(unmodified, SEQ ID NO: 17)
SYVRPLSVALGETARISCGRQALGSRAVQWYQHRPGQAPILLIYNNQDR
PSGIPERFSGTPDINFGTRATLTISGVEAGDEADYYCHMWDSRSGFSWS
FGGATRLTVLGQPKAAPSVTLFPPSSEELQANKATLVCLISDFYPGAVT
VAWKADSSPVKAGVETTTPSKQSNNKYAASSYLSLTPEQWKSHRSYSCQ
VTHEGSTVEKTVAPTECS
10-1074-y2 heavy chain
(N31K; V100dM; S100fA, SEQ ID NO: 18)
QVQLQESGPGLVKPSETLSVTCSVSGDSMNKYYWTWIRQSPGKGLEWIG
YISDRESATYNPSLNSRVVISRDTSKNQLSLKLNSVTPADTAVYYCATA
RRGQRIYGMVAFGEFFYYYSMDVWGKGTTVTVSSASTKGPSVFPLAPSS
KSTSGGTAALGCLVKDYFPEPVTVSWNSGALTSGVHTFPAVLQSSGLYS
LSSVVTVPSSSLGTQTYICNVNHKPSNTKVDKRVEPKSCDK
(mutations bolded and underlined)
10-1074-y2 light chain
(unmodified, SEQ ID NO: 17)
SYVRPLSVALGETARISCGRQALGSRAVQWYQHRPGQAPILLIYNNQDR
PSGIPERFSGTPDINFGTRATLTISGVEAGDEADYYCHMWDSRSGFSWS
FGGATRLTVLGQPKAAPSVTLFPPSSEELQANKATLVCLISDFYPGAVT
VAWKADSSPVKAGVETTTPSKQSNNKYAASSYLSLTPEQWKSHRSYSCQ
VTHEGSTVEKTVAPTECS
10-1074-y3 heavy chain
(N31K; E55D; V100dM; S100fA, SEQ ID NO: 19)
QVQLQESGPGLVKPSETLSVTCSVSGDSMNKYYWTWIRQSPGKGLEWIG
YISDRDSATYNPSLNSRVVISRDTSKNQLSLKLNSVTPADTAVYYCATA
RRGQRIYGMVAFGEFFYYYSMDVWGKGTTVTVSSASTKGPSVFPLAPSS
KSTSGGTAALGCLVKDYFPEPVTVSWNSGALTSGVHTFPAVLQSSGLYS
LSSVVTVPSSSLGTQTYICNVNHKPSNTKVDKRVEPKSCDK
(mutations bolded and underlined)
10-1074-y3 light chain
(unmodified, SEQ ID NO: 17))
SYVRPLSVALGETARISCGRQALGSRAVQWYQHRPGQAPILLIYNNQDR
PSGIPERFSGTPDINFGTRATLTISGVEAGDEADYYCHMWDSRSGFSWS
FGGATRLTVLGQPKAAPSVTLFPPSSEELQANKATLVCLISDFYPGAVT
VAWKADSSPVKAGVETTTPSKQSNNKYAASSYLSLTPEQWKSHRSYSCQ
VTHEGSTVEKTVAPTECS
10-1074-y4 heavy chain
(N31K; E55D; V100dM; S100fA, SEQ ID NO: 20)
QVQLQESGPGLVKPSETLSVTCSVSGDSMNKYYWTWIRQSPGKGLEWIG
YISDRDSATYNPSLNSRVVISRDTSKNQLSLKLNSVTPADTAVYYCATA
RRGQRIYGMVAFGEFFYYYSMDVWGKGTTVTVSSASTKGPSVFPLAPSS
KSTSGGTAALGCLVKDYFPEPVTVSWNSGALTSGVHTFPAVLQSSGLYS
LSSVVTVPSSSLGTQTYICNVNHKPSNTKVDKRVEPKSCDK
(mutations bolded and underlined)
10-1074-y4 light chain
(M90L, SEQ ID NO: 21)
SYVRPLSVALGETARISCGRQALGSRAVQWYQHRPGQAPILLIYNNQDR
PSGIPERFSGTPDINFGTRATLTISGVEAGDEADYYCHLWDSRSGFSWS
FGGATRLTVLGQPKAAPSVTLFPPSSEELQANKATLVCLISDFYPGAVT
VAWKADSSPVKAGVETTTPSKQSNNKYAASSYLSLTPEQWKSHRSYSCQ
VTHEGSTVEKTVAPTECS
(mutations bolded and underlined)

There are a number of optimizations that may increase the quality and rate of in vivo affinity maturation. First, it has been shown in in vitro selection studies that a library of HDRTs, modified at low frequency at key sites, can improve the diversification rate and accelerate discovery of potency-enhancing mutations (20). Different kinds of HDRT libraries could be used to edit primary B cells for in vivo affinity maturation. One such library could introduce wobble-codon diversity, ensuring that the distribution of hypermutation hotspots does not overly bias the sampling of sequence space. Alternatively, libraries could be generated that introduce coding changes as well. Notably, none of the 49 10-1074 mutations characterized in FIGS. 5A-5C impaired neutralization substantially. Thus, most members of a library constructed from these changes would likely maintain their ability to respond to antigens. Such a library could be used to identify combinations of these mutations that most potently bind an original antigen, or most efficiently adapt to a related antigen. Second, there is a need for higher quality antigens, adjuvants, vaccination schedules, and antigen-delivery systems. The greater immunogenicities of these antigens are important for driving initial proliferation of a library with lower average affinities (29), or broadening an antibody to bind previously resistant antigen variants. Finally, more accurate computational methods are also required to identify potency- or half-life-enhancing mutations. As described herein, every mutation that emerged in at least two individual mice was tested, but accurate predictions about the impact of a given mutation would streamline this process and accelerate the improvement of therapeutic antibodies.

In vivo affinity maturation of a known antibody is not the only application of native-loci editing. Although thorough safety testing will be necessary, edited primary B cells themselves could serve as therapeutics (30). It was shown that mice engrafted with VRC26.25-y-edited B cells, but not identically vaccinated control mice, generated potent neutralizing plasma in response to vaccination with a soluble Env trimer. Plasma from these mice regularly surpassed the potency of a 2 μg/ml plasma concentration of VRC26.25. This value exceeds the 0.75 μg/ml necessary to protect macaques from a SHIV challenge (31). Similarly. 10-1074 concentrations higher than 1.1 μg/ml delay viral rebound in SHIV-infected macaques (32), a concentration that was surpassed in some mice engrafted with 10-1074-edited B cells. Thus, engineered B cells could be employed to adaptively control the distinctive and diverse proviruses in HIV-positive persons. The adaptivity of the response depends on BCR diversity, so native-loci editing may be especially useful for suppressing and perhaps diminishing the scale of the HIV reservoir. It is also likely that multiple Env epitopes must be targeted to ensure that viral suppression is consistent and lasting. It has been formally shown that B cells edited to express 10-1074 can expand and generate neutralizing plasma alongside those edited to express VRC26.25-y, with both sets of cells responding to the same antigen. Thus, individuals might receive B cells engineered to mature and produce different bNAb classes. Of course, engineered B cells capable of affinity maturation could be employed for other infectious diseases, for example enhancing the control of pathogens in immunocompromised persons. Even when affinity maturation is not critical—for example when targeting a relatively fixed tumor antigen-engineered B cells could provide long-term antibody expression without the immune clearance seen with gene-therapy delivery systems (33, 34). The model developed here can also facilitate conventional vaccine studies, including evaluation of Env-based vaccines. A key question for any antigen is whether it retains its critical epitopes in vivo, and whether those epitopes are an important focus of the immune response. Efficient expansion and maturation of B-cells expressing progenitor or mature bNAbs could be used to determine if the bNAb epitope is intact and immunologically dominant. Finally, this model can also be used to address some outstanding questions in immunology, including the determinants of somatic hypermutation and affinity maturation, sources of tolerance to hypermutated BCRs, the relationship between antigen affinity and B-cell proliferation, the best strategy to broaden a response, and the upper limits of affinity achievable in vivo. As such, the disclosed approach may be used to improve the properties of therapeutic antibodies, develop new B-cell based therapies, test conventional vaccines, and address previously inaccessible questions in B-cell biology.

### Materials and Methods

### AAV Production, Purification, and Quantification

HEK293T cells from ATCC (CRL-3216) were cultured in DMEM with 10% FBS at 37° C. in 5% CO2. AAV6 and AAV-DJ vectors were packaged in HEK293T cells. In brief, a plasmid encoding the AAV rep and cap genes, a plasmid encoding adenoviral helper genes, and a plasmid encoding desired HDRT flanked by AAV ITRs were mixed and transfected into 293T cells using polyethylenimine (PEI, Polysciences, 24765-1). All plasmids including the AAV ITR were confirmed by agarose gel analysis and sequencing. Culture media was changed 16-24 hours after transfection, and AAV was harvested after an additional 48 hours. AAV was purified with the AAVpro Purification Kit (Takara, 6666) according to manufacturer instructions, and concentrated into PBS. Quantification was performed by real-time PCR with AAVpro Titration Kit Ver.2 (Takara, 6233) according to manufacturer's instructions.

dsDNA HDRTs Preparation

dsDNA templates containing 5′ and 3′homology arms were generated from AAV plasmids or synthesized gBlocks (IDT) through PCR using PrimeSTAR Max DNA Polymerase (Takara, R045A). Forward primers contain 5′ phosphate groups, and reverse primers were stabilized with two phosphorothioate-stabilized DNA bonds at the 3′ end. Amplicons were purified with SPRI selection (1×) and concentrated into desired volumes with RNase-free water.

### Human Cells Culture and Electroporation

The human B-cell lymphoma cell line Jeko-1 was purchased from ATCC (CRL-3006). These cells were cultured in RPMI1640 GlutaMAX media, with 10-20% FBS and penicillin-streptomycin at 37° C. in 5% CO2. Cell density was kept between 0.5-2.0×106 cells/ml. Culture medium was changed to medium without penicillin-streptomycin before electroporation. For optimal editing. 3.6 million Jeko-1 were washed once with PBS and concentrated into 80 μl electroporation buffer (Lonza, V4XC-3024). RNP complexes were prepared using 4.5 μl 100 μM gRNA, 1.12 μl 250 μM Mb2Cas12a, 3.12 μl PBS, and 1.26 μl 1M NaCl and incubated at RT for 15 min. 10 μg ds-HDRTs with 2 μl Cas12a enhancer (IDT, 1076301) were then added and incubated for an additional 1-2 minutes. Cells were then mixed with RNP+HDRT and transferred to 100 μl nucleocuvette vessels, then electroporated using a Lonza 4D nucleofector CA-137 program according to manufacturer's instructions. After electroporation, cells were incubated at RT for 10 minutes before transfer into 6-well plates containing antibiotics-free medium with 20% FBS. For AAV mediated electroporation, 0.2-1.0×105 MOI of AAV6 HDRT were incubated with cells in serumfree medium for 6 hours at 37° C. (37-39). Cells were then electroporated with assembled RNP and returned to AAV6 containing medium no later than 5 minutes after electroporation. Editing efficiency was analyzed 48 hours following electroporation.

### Animals

Mice studies were approved and carried out in accordance with protocols provided to the Institutional Animal Care and Use Committee (IACUC) at Scripps Research (Jupiter, FL) under approval number 17-026. All experiments were performed in 6-10 weeks female C57BL/6J (CD45.1 or CD45.2) mice. Mice were housed at ambient temperature and humidity on a 12 hour light cycle. No more than 5 mice or less than 2 mice were housed together. All procedures were performed on animals anesthetized using isoflurane.

### Murine B Cells Isolation and Activation

Murine B cells were obtained from CD45.1 mice spleen. In brief, spleens were mechanically dissociated, and cells were forced through a 70 μm cell strain into 2% FBS containing RPMI medium. After red blood cell lysis for 3 minutes, B cells were enriched by negative selection (Miltenyi, 130-090-862) according to manufacturer's instructions. After that, B cells were cultured in RPMI medium supplemented with 1×NEAA, 1× sodium pyruvate, 10 mM HEPES, 53 M 2-mercaptoethanol. 10% FBS, and activated with 4 μg/ml anti-mouse RP105 antibody (Biolegend, 117710) for 30-32 hours.

### Murine B Cells Electroporation

Activated cells were washed twice with PBS and resuspended in 75 μl electroporation buffer (Lonza, V4XP-4024). For RNP preparation, 4.5 μl 100 μM gRNA, 1.12 μl 250 UM Mb2Cas12a, 3.12 μl PBS, and 1.26 μl 1M NaCl were mixed and incubated at RT for 15 minutes, 20 μg dsDNA as HDRTs and 2 μl Cas 12a enhancer were then added and incubated for an additional 1-2 minutes. Cells were then mixed with RNP+HDRT and transferred to 100 μl nucleocuvette vessels, then electroporated using a Lonza 4D nucleofector DI-100 program according to manufacturer's instructions. When an AAV donor was used, cells were washed twice with PBS and resuspended in 0.5-1.2×106 MOI AAV-DJ containing serum-free medium for 1 hour incubation at 37° C. Cells were then electroporated with assembled RNP, and put back to AAV-DJ containing medium no later than 5 minutes. After 1 hour, FBS and activation components, namely anti-mouse RP105 antibodies, were added back into the medium for further culture.

### Adoptive Transfer of B Cells

18-19 hours after electroporation, engineered cells were wash twice with cold PBS, then filtered, and resuspended in 200 μl PBS/mouse containing 3-8 million cells according to editing efficiency. The same number of edited-cells were transferred between native-loci and intron methods. The cell suspension was injected intravenously into age-matched CD45.2 mice via the retroorbital sinus. A small proportion of B cells were further cultured ex vivo, in RPMI 10% FBS medium supplemented with LPS (5 μg/ml) mouse IL-4 (10 ng/ml), and anti-mouse RP105 antibody (2 μg/ml). These cells were used to analyze editing efficiency by flow cytometry 3 days after electroporation.

### Protein Production and Purification

Expi293 cells were resuspended at a density of 3×106 cells/ml. For immunogen production, the constructs of CRF250.SOSIP.v7 and BG505 13-01 were transfected into Expi293F cells or Expi293 GnTI-cells. SOSIP constructs, furin, FGE (formylglycine generating enzyme), PDI (protein disulfide isomerase) were co-transfected at 4:1:1:1 ratio with FectoPRO. Supernatants were harvested 5 days after transfection, filtered, and purified with PGT145 affinity columns. Proteins were eluted with gentle Ag/Ab elution buffer (Thermo, 21027). The elution was exchanged with buffer (10 mM HEPES, 75 mM NaCl pH 8.0) and concentrated in 30K Amicon Ultra-15 filter tubes. Some purified SOSIPs were conjugated with fluorescence by Lightning-Link Antibody Labeling Kits according to manufacturer's instructions. Alternatively, some purified SOSIPs were biotinylated with EZ-Link Sulfo-NHS-Biotin (Thermofisher, 21217) and then incubated with fluorescent streptavidin for use in flow cytometry. Other trimers were further purified by SEC on a Superdex 200 Increase 10/300 GL column for animal immunizations. 4 I3-01 constructs and PDI were co-transfected at 4:1 ratio with FectoPRO. Supernatants were harvested 5 days after transfection, filtered, and purified with CH01 affinity columns. The purity of 13-01 nanoparticles was characterized by SEC on a Superose 6 Increase 10/300 GL column for animal immunizations. For antibody production. Expi293 cells were co-transfected with plasmids encoding heavy chains and light chains at 1:1 ratio, or with the third plasmid, human TPST2 (tyrosine-protein sulfotransferase 2, for VRC26.25 and its variants) at 2:2:1 ratio with FectoPRO transfection reagents. 4-6 days post-transfection, supernatants were collected, filtered, and purified through HiTrap Mabselect SuRe columns (GE Healthcare Life Sciences). Columns were washed and eluted with IgG elution buffer (Thermo, 21004), and pH values were adjusted with neutralization buffer (1M Tris-HCl, pH 9.0). The elution was buffer exchanged and concentrated with PBS in 30K Amicon Ultra-15 filter tubes.

### Mouse Immunization

75 μg soluble CRF250 SOSIPs or 20 μg 13-01 nanoparticles were mixed with 25 μg MPLA (Invivogen, vac-mpls) and 10 μg QuilA (Invivogen, vac-quil) as adjuvants in 250 μl PBS/mouse by subcutaneously (s.c.) and intraperitoneally (i.p.) administration. A total of four s.c. injection locations, which drain to lower and upper lymph nodes, and one i.p. injection were administered in a volume 50 μl per site. The first immunization was administered 24 hours after adoptive transfer of edited cells, and mice were then immunized every 3 weeks.

### Blood Collection and PBMC Preparation

Blood samples were collected through submandibular bleeding of mice into EDTA-coated tubes at the time intervals indicated in FIG. 2A. All procedures were done on isoflurane-anesthetized mice. Samples were spun at 1200 g for 10 minutes to separate plasma and heat-inactivated at 56° C. for 30 minutes. Another centrifuge step at 2000 g for 10 minutes removed precipitates from plasma, and stored at −80° C. The remaining cells were diluted with an equal volume of PBS and overlaid on Lymphoprep (STEMCELL Technologies, 07801) in 15 ml SepMate tubes (STEMCELL Technologies, 85415) to separate PBMCs. After red blood cell lysis, lymphocytes were again washed with PBS, and analyzed by flow cytometry or lysed for RNA isolation.

### Flow Cytometry

Cells were counted to achieve target cell numbers, and washed once with FACS buffer (PBS, 2% FBS, 1 mM EDTA). Cells were stained for 20-30 minutes on ice with fluorescently labeled SOSIPs or antibodies in volumes of 100 μl per 1×106 cells and washed again with FACS buffer. Biotinylated SOSIPs were incubated with streptavidin-488/647 at RT 40 minutes before use. Cells were gated according to background levels, determined as the binding percentage in unedited cells with the same SOSIP at the same concentration. Single live cells were analyzed on BD Accuri C6 flow cytometer or Beckman Coulter Gallios Flow Cytometer.

Isolation of B Cells from Spleen, Lymph Nodes, And Bone Marrow

Spleen and superficial lymph nodes, such as inguinal, axillary, brachial, and cervical lymph nodes, were mechanically dissociated, and the cells were forced through a 70 μm cell strain into 2% FBS containing PRMI medium. Bone marrow was flushed out from tibia and femurs using a 25-G needle and a syringe with cold-medium and driven through a 70 μm cell strain. After red blood cell lysis for 3 minutes and a second filtration step, cells were resuspended with MACS buffer in 100 5 μl/107 cells (0.5% BSA, 2 mM EDTA in PBS). 105 cells were kept for flow cytometry analysis, and others were used for CD45.1-positive isolation. In brief, cells incubated with 5 μl mouse Fc blocker per 107 cells for 5 minutes. Cells were then incubated with 1 μl biotin-CD45.1 antibody per 107 cells for 30 minutes. Cells were washed with 1-2 ml MACS buffer/107 cells, resuspended in 70 μl/107 cells, and incubated with 20 μl anti-biotin microbeads ultrapure (Miltenyi, 130-105-637) for 15 minutes. The suspension was again washed and filtered to remove any pellet formed during this process. Cells were then resuspended in 500 μl MACS buffer and positively selected through LS columns twice to collect CD45.1+ cells for RNA isolation.

Next Generation Sequencing Analysis (NGS) of Ig mRNA

RNA was isolated from approximately 1-5 million sorted CD45.1+ cells with the RNeasy micro kit (Qiagen, 74004). Heavy-chain cDNA synthesis was performed with 8 μl RNA with 10 pmol of primers targeting constant region of IgM (CTG GAT GAC TTC AGT GTT GT (SEQ ID NO: 22)), IgA (CCA GGT CAC ATT CAT CGT G (SEQ ID NO: 23)) or IgG1/2 (KKA CAG TCA CTG AGC TGC T (SEQ ID NO: 3)), IgG3 (GTA CAG TCA CCA AGC TGC T (SEQ ID NO: 4)) in 20 μl total reaction with Superscript III reverse transcriptase (Thermo) using the manufacturer's protocol. Remaining dNTPs were removed with ExoSAP-IT (Thermo). The entire treated PCR products then were added with 10 pmol the heavy chain-specific primer (AGA CGT GTG CTC TTC CGA TCT NNT ACN NNN NNA GTN NNN NNG TGT CCA CTC CCA AGT GCA GCT G (SEQ ID NO: 5) for 10-1074 and AGA CGT GTG CTC TTC CGA TCT NNT ACN NNN NNA GTN NNN NNA GGT GCA GTT GGT GGA GTC TGG (SEQ ID NO: 6) for VRC26.25-y) with HotStar Taq plus polymerase. The primers contain unique molecular identifiers (UMI) and Illumina adaptor sequences were incorporated during this round of PCR. Residual primers and dNTPs were removed with ExoSAP-IT treatment, and dsDNA was purified with SPRI beads (0.8×). A second round PCR was performed using a 5′ nested primer (GTG ACT GGA GTT CAG ACG TGT GCT CTT CCG AT*C (SEQ ID NO: 7)) and 3′ UMI containing nested primer mix (IgM: ACA CTC TTT CCC TAC ACG ACG CTC TTC CGA TCT (NN)2-6 GGG GGA AGA CAT TTG G (SEQ ID NO: 8); IgG1/2: ACA CTC TTT CCC TAC ACG ACG CTC TTC CGA TCT (NN) 2-6 AGT GGA TAG ACM GAT G (SEQ ID NO: 9); IgG3: ACA CTC TTT CCC TAC ACG ACG CTC TTC CGA TCT (NN) 2-6 AAG GGA TAG ACA GAT G (SEQ ID NO: 10); IgA: ACA CTC TTT CCC TAC ACG ACG CTC TTC CGA TCT (NN) 2-6 TCA GTG GGT AGA TGG TG (SEQ ID NO: 11)) in a 50 μl reaction volume (Q5 hifi, NEB). The PCR products were cleaned with dual side SPRI selection (0.55×/0.73×). Final products were confirmed on 1% agarose gel and were indexed with NEBNext® Multiplex Oligos for Illumina (Dual Index Primers Set 1) and cleaned again with dual side SPRI selection (0.55×/0.73×). Indexed fragments were pooled and sequenced on Illumina Miseq to obtain 2×300 bp read chemistry. Light chain preparation is the same as heavy chain, except for changed IgK primers below. IgKRT (ACT GCC ATC AAT CTT CCA C (SEQ ID NO: 12)), VRC26.25-y light-chain primer and 10-1074 light-chain primer (AGA CGT GTG CTC TTC CGA TCT NNT ACN NNN NNA GTN NNN NNT GTT CTA ACC CAA CCT CCC TCT G (SEQ ID NO: 13) and AGA CGT GTG CTC TTC CGA TCT NNT ACN NNN NNA GTN NNN NNA AAC CAA CGG TTC CTA TGT CAG G (SEQ ID NO: 14)), 3′ UMI containing nested primer mix (IgK: ACA CTC TTT CCC TAC ACG ACG CTC TTC CGA TCT (NN) 2-6 GGA TGG TGG GAA GAT G (SEQ ID NO: 15)).

### Pseudovirus Production and Neutralization Assays

HEK293T cells in antibiotics-free medium were co-transfected with the plasmid expressing Env of desired isolates and pNL4.3ΔEnv plasmid at 1:1 ratio with PEIpro reagents. After 48 hours, supernatants were collected, filtered, and stored at −80° C. TZM-bl neutralization assays were performed as previously described (40). Briefly, titrated antibodies in 96-well plates with tips change were incubated with pseudotyped viruses at 37° C. for 1 hour. TZM-bl cells were then added to the virus/inhibitor mix using 10,000 cells/well. Cells were then incubated for 48 hours at 37° C. Viral entry was determined by luciferase readout with BriteLite Plus (Perkin Elmer, 6066761), quantified with a Victor X3 plate reader (Perkin Elmer). IC₅₀ and IC₈₀ values were calculated using the nonlinear regression model in GraphPad Prism 9.

### Immunofluorescence Assay (IFA) on HEp-2 Cells

IFA was performed with ANA HEp-2 Test Kits from Zeus Scientific (FA2400EB) according to manufacturer's instructions. Briefly, 100 μg/ml antibodies and controls were incubated on HEp-2 cell slides at RT for 40 minutes, and then washed 3 times with PBS. FITC-conjugated anti-human IgG antibodies were coated to each well at RT for 25 minutes, and slides were again washed. Slides were viewed using a Leica DMIL LED microscope at a 292 ms exposure, and intensity was measured by Image J.

### Example 3. Heavy-Chain CDR3-Engineered B Cells Facilitate In Vivo Evaluation of HIV-1 Vaccine Candidates

HIV-1 infected persons can raise potent broadly neutralizing antibodies (bnAbs) (43-45), but to date no vaccine strategy can do so consistently. These bnAbs nonetheless serve as important guides to the design of HIV-1 immunogens by identifying conserved target epitopes and highlighting features of B-cell receptors (BCRs) necessary to recognize these epitopes (46-48). Unfortunately, these features often include hard-to-elicit substitutions, insertions, and deletions that emerge only after years of active infection. Thus, sequential immunization strategies are presumed necessary to elicit bnAbs recognizing the CD4-binding site and V3-glycan epitopes of Env (49-52). However, another class of bnAbs, those recognizing the V2-glycan/apex epitope, have qualitatively different properties that may simplify their elicitation (53-56). For example, apex bnAbs require relatively less somatic hypermutation (SHM) and much of their binding energy is localized to their heavy-chain complementarity-determining 3 regions (HCDR3s). These HCDR3s are unusually long, acidic, and tyrosine-sulfated (54, 55). Antibodies with similarly long HCDR3s and tyrosine-sulfation motifs are readily observed in the repertoires of HIV-1 negative persons (57), and these antibodies might serve as apex bnAb precursors. Consistent with this supposition, apex bnAbs emerge more quickly and frequently after infection than other bnAb classes (58-60). Moreover, although their breadth is relatively limited, apex bnAbs are typically more potent than other bnAbs (53, 61-63). Thus, the apex epitope remains a critical target of efforts to develop an effective HIV-1 vaccine.

Apex bnAbs such as PGT145 and VRC26.25 also serve an additional function in vaccine design. Specifically, because they recognize only properly assembled Env trimers in a closed conformation (54, 64, 65), they help purify (66, 67) and quality control (68) candidate Env antigens. Env trimers in this closed state hide otherwise immunodominant non-neutralizing epitopes and ensure that key neutralizing epitopes are presented as they are on functional virions. Hence, considerable efforts have been applied to increasing the stability and immunogenicity of soluble, trimeric Env antigens, building on SOSIP (49, 69-74) and native-flexible linked (NFL) architectures (75-77), among others (78, 79). Rabbits and macaques immunized with these trimers can elicit autologous and weak heterologous neutralizing responses, sometimes including detectable anti-apex responses (49, 56, 80-82).

Efforts to design and improve Env immunogens that maintain this closed apex-bnAb-binding conformation nonetheless face challenges. The quaternary apex epitope is difficult to maintain during protein purification, and nearly impossible to monitor in vivo. The apex bnAbs PGT145(66, 67) or VRC26.25 (83) are used to enrich for properly assembled trimers, but the structural integrity of these trimers can vary during subsequent handling or after immunization. The emergence of mRNA vaccines can bypass the need for protein in vitro handling (84-86), but in vivo stability remains a key variable in antigen design. However, the stability of these antigens is not the only determinant of their ability to raise potent bnAbs. For example, immunodominant non-neutralizing epitopes can compete with neutralizing ones (69, 87, 88), cellular and serum proteases can destroy key epitopes, non-neutralizing antibodies can drive disassembly of trimeric antigens, and unanticipated interactions with host-cell proteins or extracellular matrix elements can limit their access to follicular dendritic cells (89, 90).

Thus, efficient and physiologically relevant in vivo systems for measuring anti-apex responses will be critical for developing better Env antigens. However, current wild-type rodent, rabbit, or macaque models are not optimal in large part because the diversity (D) gene segments, key contributors to the HCDR3, are highly species-specific (91, 92). Transgenic mice can be engineered to express mature or progenitor apex bnAbs (93, 94), but these mice are slow to generate or modify, and their antigen reactive repertoires are essentially monoclonal, biasing antigen comparisons. Strategies to engineer mature murine B cells to express human bnAbs and adoptively transfer these cells into wild-type mice have been developed for novel cell-based therapies (56, 95-100). These engineered B cells proliferate in response to antigen and generate neutralizing sera, but this response has impaired somatic hypermutation, and is monoclonal and therefore unrepresentative of a human repertoire. Thus, there remains a need for a robust, sensitive, and adaptable system for monitoring the stability and immunogenicity of Env trimers in vivo.

It is demonstrated herein that a diverse repertoire of murine BCRs engineered to express the HCDR3s of several apex bnAbs can bind soluble Env trimers. When B cells modified to express the VRC26.25 HCDR3 were introduced into wild-type mice, they proliferated, class switched, hypermutated, and generated potent neutralizing sera following immunization with a range of Env immunogens. Notably, these engineered B cells affinity matured, as indicated by the ability of hypermutated HCDR3 to improve the potency of wild-type VRC26.25. Using this system, multiple SOSIP variants were evaluated in vivo for their antigenicity. Among them, a version of the chimeric SOSIP protein raised the most potent apex-antibody responses and relatively few non-neutralizing antibodies. Responses to these SOSIP protein variants were markedly enhanced when they were delivered as an mRNA vaccine and expressed as transmembrane proteins (SOSIP-TM). This more immunogenic vaccine candidate induced neutralizing responses from B cells expressing the HCDR3 of the apex bnAbs PG9 and PG16, as well as affinity maturation of the predicted unmutated common ancestor (UCA) (61) of the VRC26.25 HCDR3. Thus, the approach taken here can accelerate development of stable Env immunogens that elicit apex bnAbs from human HCDR3 precursors.

Mice Engrafted with HCDR3-Edited B Cells Generate Neutralizing Sera after Immunization.

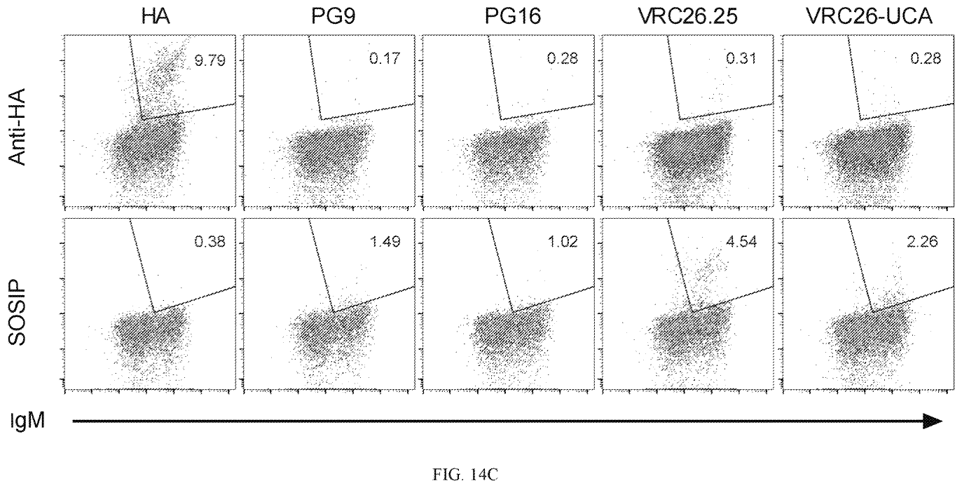

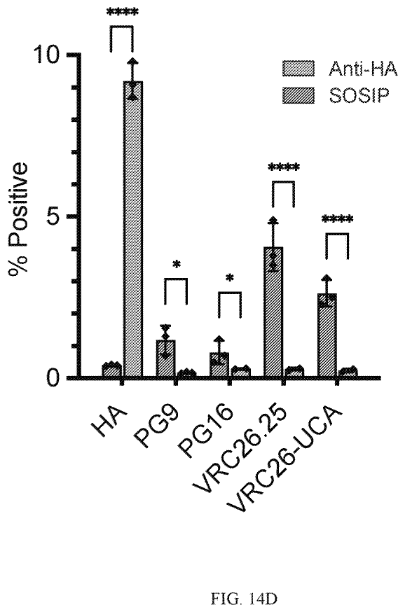

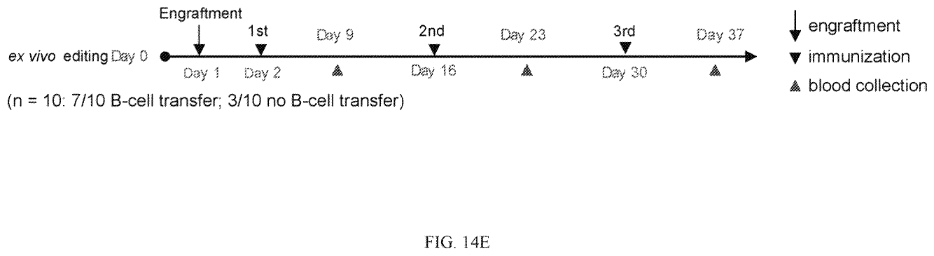

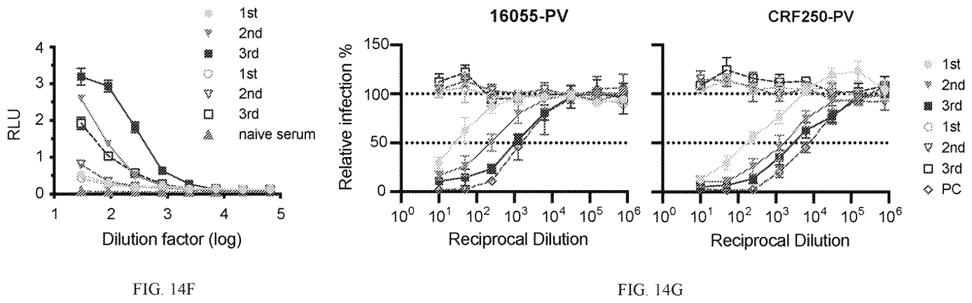

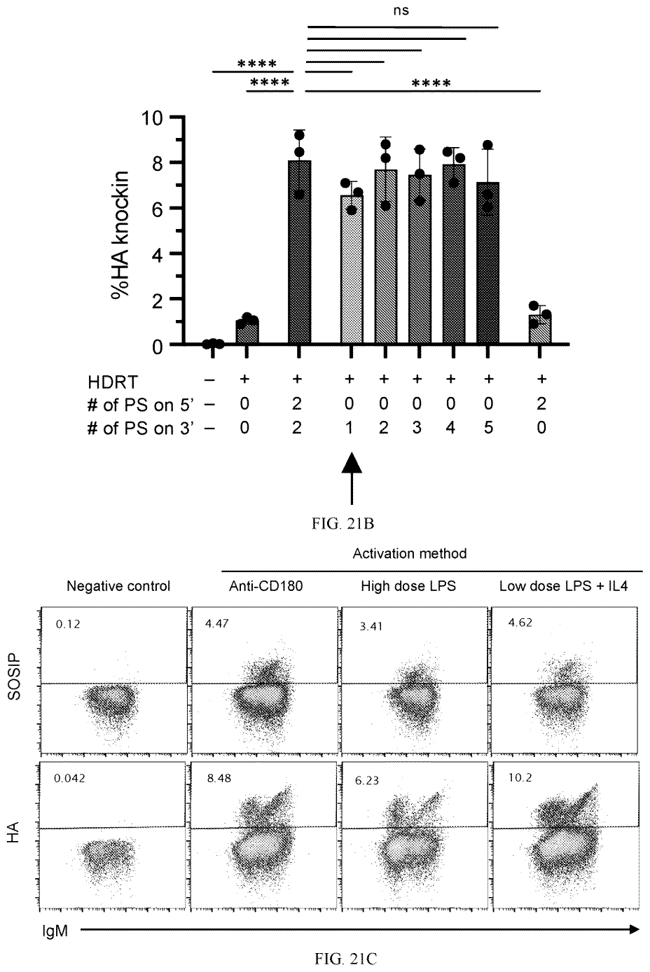

It has been previously demonstrated that primary human B cells can be edited to express an exogenous HCDR3 as part of an otherwise diverse native human antibody repertoire (101). Specifically, techniques to introduce the HCDR3s of the apex bnAbs PG9 and PG16 into naïve mature B-cell receptors encoded by VH-1, VH-3, and VH-4 families of variable genes were developed. Reflecting the high dependence of apex bnAbs on their extended, usually tyrosine-sulfated, HCDR3s (FIG. 14A), it was shown that B cells engineered to express these HCDR3s bound SOSIP antigens in the context of diverse human heavy- and light chains. These initial studies in human cells suggested that B cells so edited might expand and affinity mature in vivo in response to HIV-1 immunogens. To test this hypothesis, primary murine B cells were modified using the same strategy. Specifically, splenic B cells were electroporated with ribonucleoproteins (RNP) composed of the Mb2Cas 12a CRISPR effector protein and guide RNA (gRNA) complementary to a region within the JH4 segment (FIG. 14B). Mb2Cas12a efficiently recognizes a convenient GTTC PAM present in a usually conserved region of JH4, allowing it to cut a site near the DJ junction. A short single-stranded homology-directed repair template (HDRT), with a 5′ homology arm complementing a ˜70-nucleotide consensus murine VH1 sequence and a 3′ homology arm complementing a ˜60-nucleotide 3′-region of JH4 and its adjacent intron, was used to introduce the full HCDR3 of several apex bnAbs, or a hemagglutinin (HA) tag sequence, into the VDJ-recombined murine variable gene. HDRTs were modified with two 3′ phosphorothioates that markedly enhanced editing efficiency (FIG. 21A-B). B cells were activated with LPS (97), which enhanced editing efficiencies comparably to other reported activation protocols (95, 99) (FIG. 21C). Using this approach, the HCDR3 regions of the apex bnAbs VRC26.25, PG9. PG16, and the predicted unmutated common ancestor (UCA) of the VRC26-family (VRC26-UCA) were introduced into primary mouse B cells and measured their abilities to bind an HA-antibody control or a SOSIP trimer (FIG. 14C-D). B cells edited to express the HA-tag as a HCDR3 were used as negative controls. It was observed that this SOSIP trimer specifically bound B cells edited to express all four apex-bnAb-derived HCDR3s, and that it bound cells expressing the VRC26.25 HCDR3 most efficiently.

To determine whether these cells could respond to antigen in vivo, B cells were isolated from the spleens of B6 CD45.1 mice, edited them as above to introduce the VRC26.25 HCDR3 region, and adoptively transferred to wild-type (CD45.2) C57BL/6J mice. Recipient mice initially received 30 million cells and then were immunized with adjuvanted SOSIP trimers conjugated to a mi3 60-mer scaffold (102, 103) according to the schedule represented in FIG. 14E. Serum harvested one week after each of three immunizations was characterized for its ability to bind SOSIP trimers (FIG. 14F) and to neutralize pseudoviruses presenting the Envs of the CRF250 and 16055 isolates (FIG. 14G). It was observed that mice produced increasing SOSIP-binding antibodies with each immunization. The neutralization efficiency increased similarly in mice engrafted with edited B cells. In contrast, mice that did not receive edited cells, but which were immunized on the same schedule, did not raise neutralizing sera, indicating that edited B cells were responsible for the neutralizing activity.

### HCDR3-Edited B Cells Migrate to Germinal Centers, Class Switch, and Hypermutate.

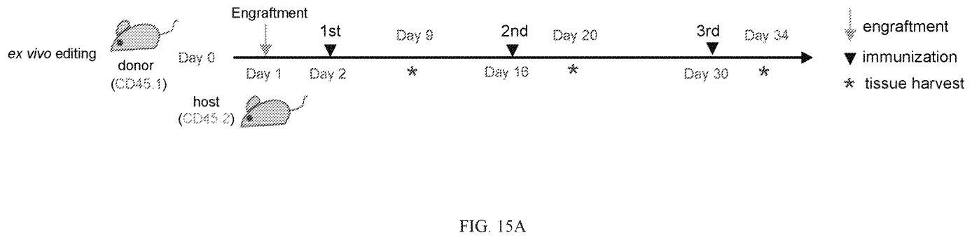

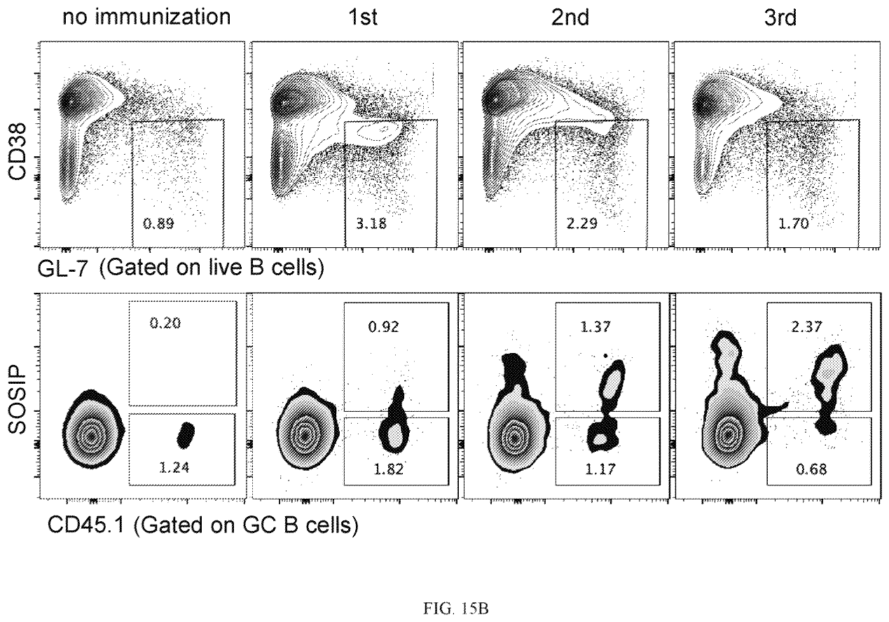

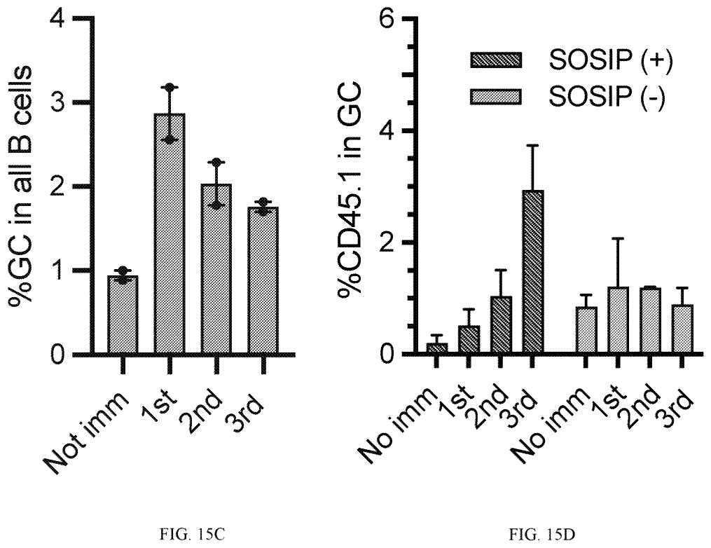

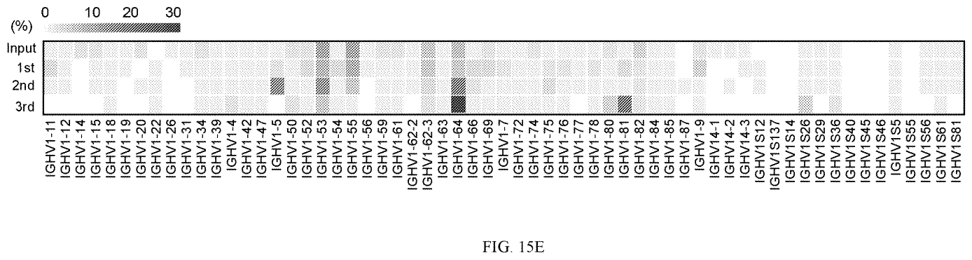

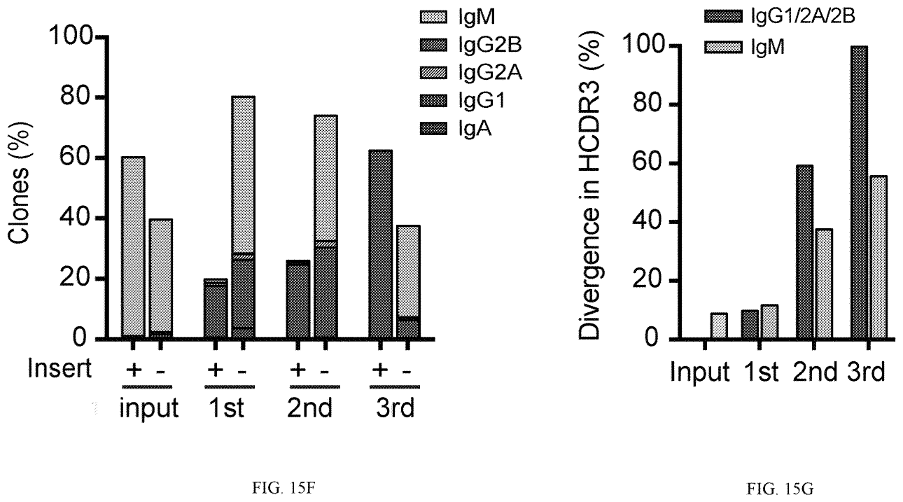

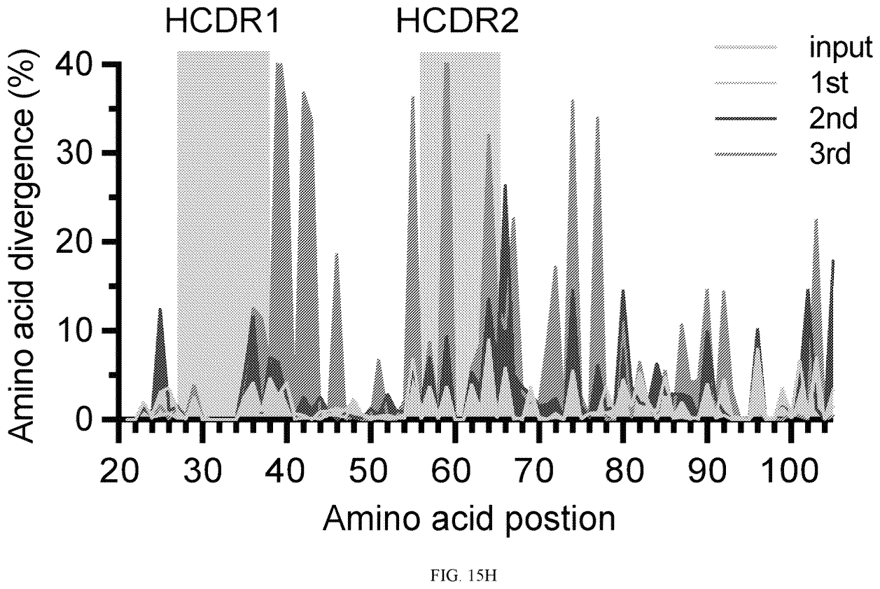

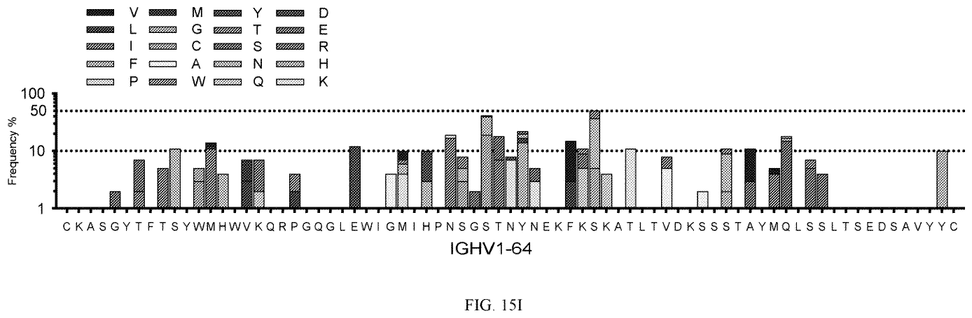

It was next sought to determine if edited B cells migrated to germinal centers in the lymph nodes and spleens of recipient mice. An experiment similar to that shown in FIG. 14 was performed (FIG. 15A) except that spleens and lymph nodes were harvested from two mice after each of three immunizations, and control mice were also engrafted with cells expressing the VRC26.25 HCDR3, but not immunized. An increase in germinal center (CD38−/GL-7+) B cells was observed, including both donor CD45.1+ cells and recipient CD45.1-cells, compared to non-immunized mice (FIGS. 15B and C). The percentage of SOSIP binding donor cells [SOSIP(+)/CD45.1+] increased after each immunization, whereas the CD45.1+/SOSIP(−) cells did not (FIGS. 15B and D), suggesting that only antigen-reactive cells proliferated in germinal centers.

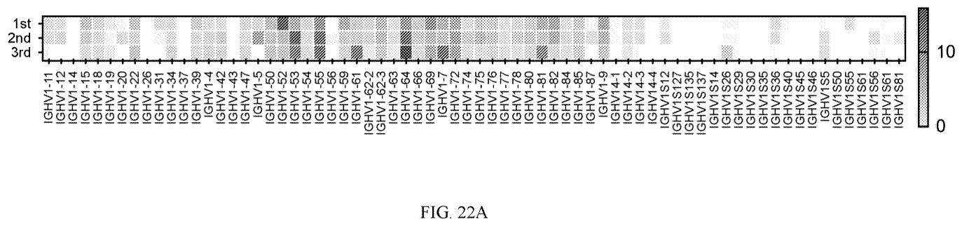

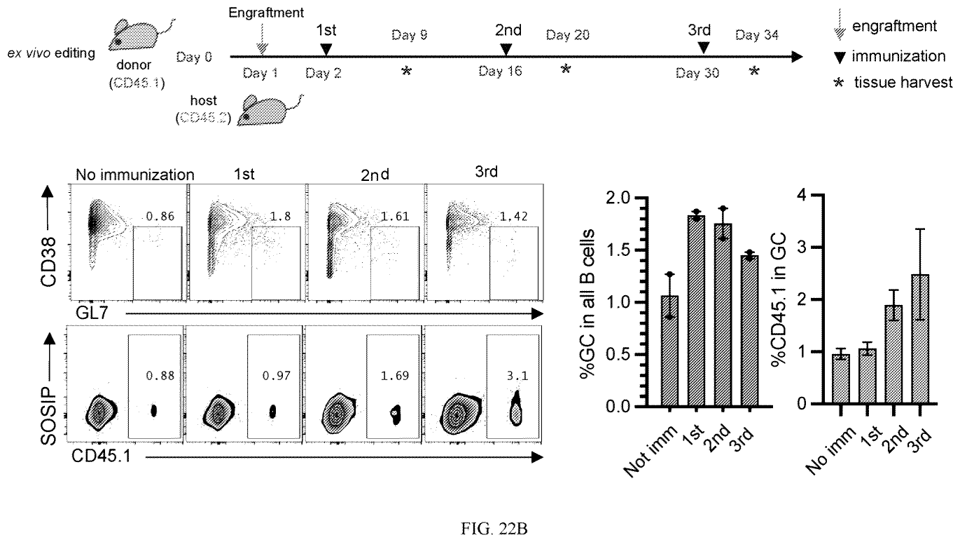

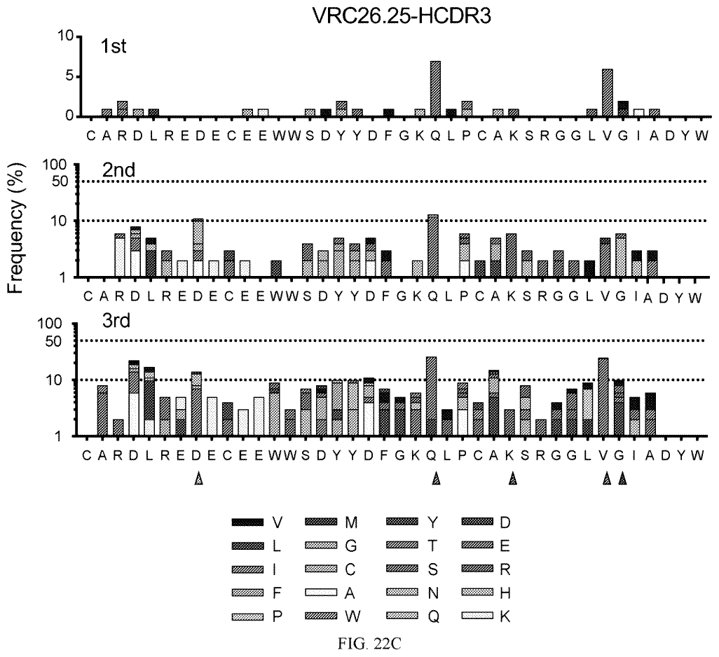

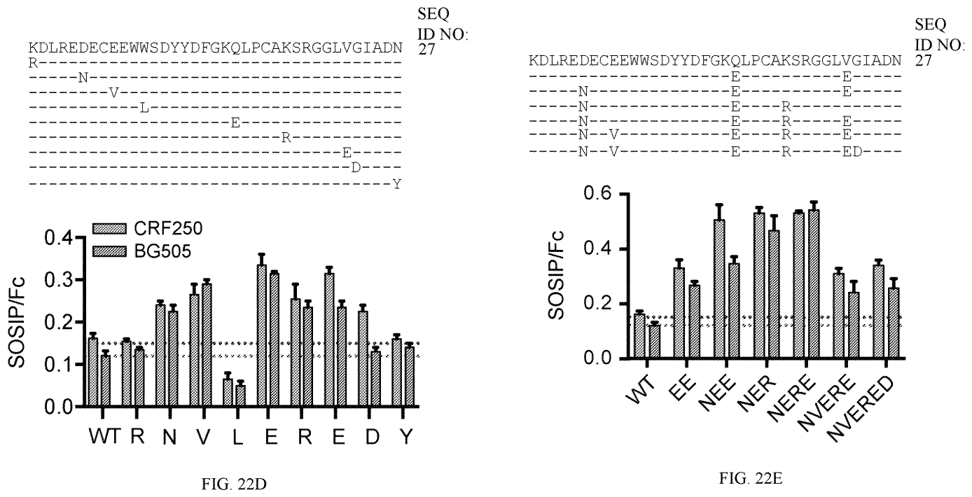

Using next generation sequencing (NGS), the heavy chain diversity of VRC26.25 HCDR3-edited B cells in lymph nodes and spleens of each mouse group (FIG. 15E) was also investigated. It was observed that this HCDR3 was present in murine BCRs encoded by many VH1-family segments, and that the original distribution of successfully edited VH1 genes was largely reflected in immunized mice. An enrichment of cells with BCRs from VH1-64, and -81 families over successive immunizations was further observed. Similar enrichment was also observed in unedited donor B cells (FIG. 22A), suggesting that this enrichment is independent of apex recognition. It can be concluded that the repertoire diversity of HCDR3-edited B cells is largely preserved in vivo after multiple immunizations, consistent with the predominant contribution of the VRC26.25 HCDR3 to Env association.

Class-switching and hypermutation frequencies of HCDR3-engineered BCRs were also investigated. It was observed that the number of class-switched CD45.1 donor cells expressing the VRC26.25 HCDR3, but not those without this insert, increased with immunization, and that the majority of these edited cells switched to the IgG1 isotype (FIG. 15F). The number of HCDR3s with one or more amino acid mutations also increased with immunization in both IgM and IgG, with more mutations associated with IgG isotypes (FIG. 15G). The frequency of framework, HCDR1, and HCDR2 mutations also increased with immunization in HCDR3-edited donor cells (FIG. 15H), with diverse amino acid changes emerging at multiple sites (FIG. 15I). Collectively, these data show that HCDR3-edited B cells largely maintain their underlying diversity while they proliferate, class switch, and hypermutate in response to immunization.

### Affinity Maturation of HCDR3-Edited B-Cell Receptors.

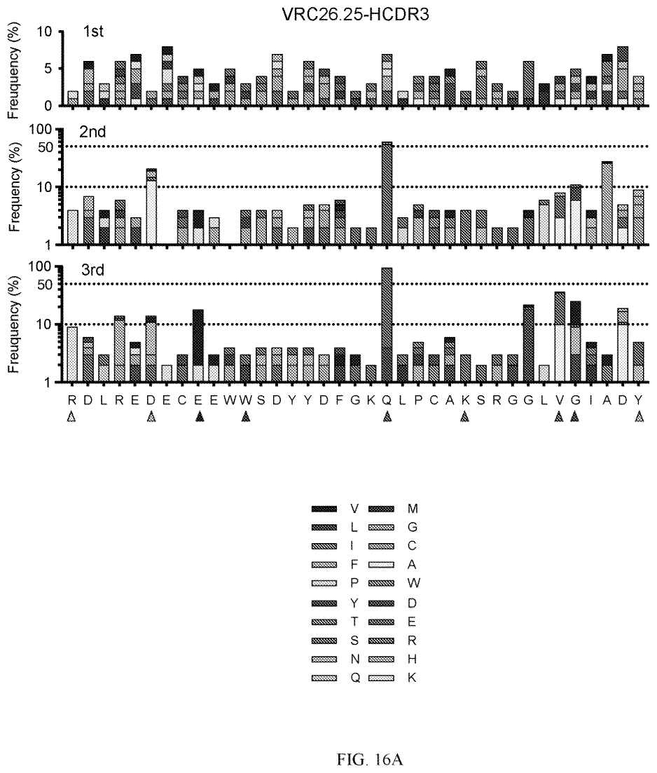

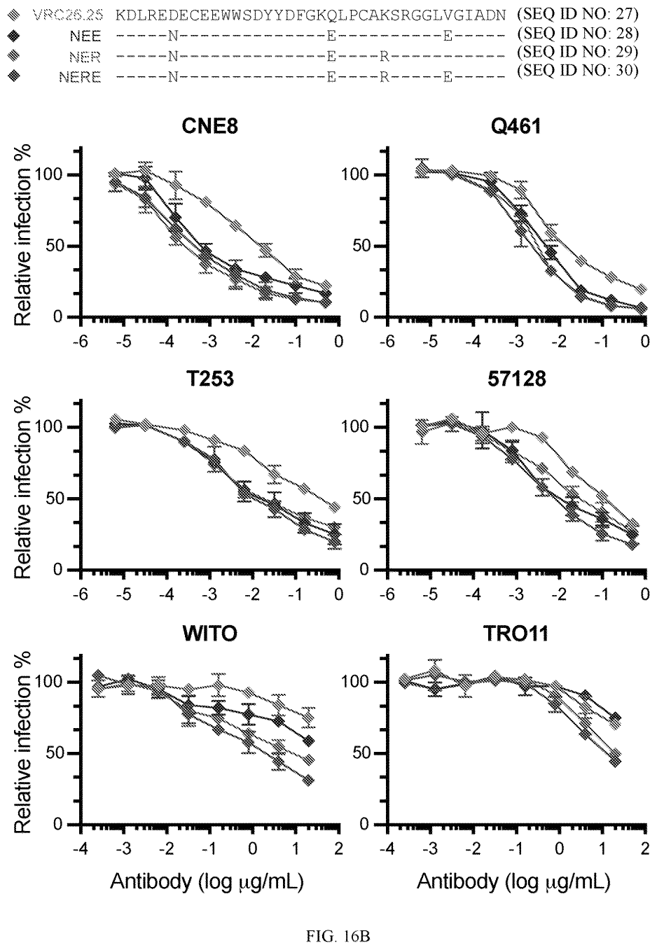

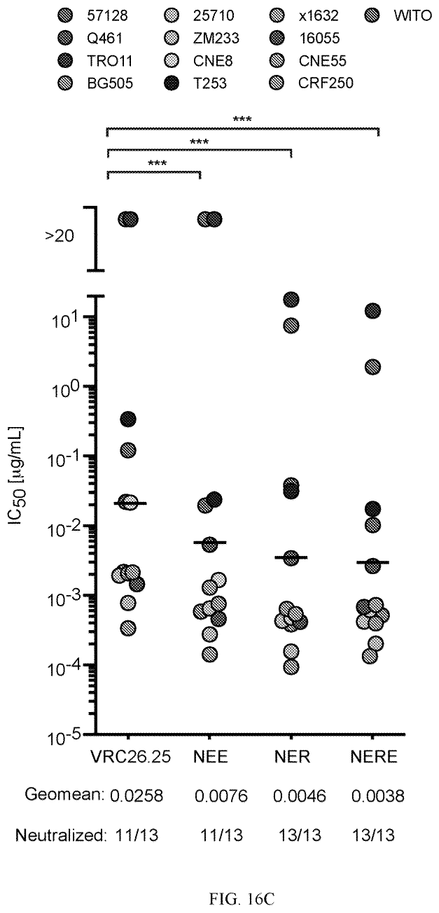

Mutation patterns in the engineered HCDR3 regions after each immunization were analyzed (FIG. 16A), and again repeated amino acid changes were observed at multiple locations, and most notably a Q100nE mutation. This mutation was observed in other VRC26 variants from the human donor CAP256 and was previously shown to improve the breadth and potency of VRC26.25 (61, 104). This mutation emerged in all six mice that were characterized, including those immunized only once, and the frequency was increased by 25- or 45-fold after receiving two or three immunizations, respectively. This observation was also repeated in six additional mice in a parallel experiment using a keyhole limpet hemocyanin (KLH) carrier protein to present SOSIP proteins (FIG. 22B), which are less immunogenic due to the inconsistent conjugate orientations. Individual HCDR3 mutations that emerged in all three groups of mice (repeated mutations indicated with triangles in FIG. 16A) were analyzed and it was observed that five of nine characterized mutations increased binding of VRC26.25 to SOSIP proteins generated from two HIV-1 isolates (FIG. 22C). Combinations of these mutations further increased their binding, with highest SOSIP-binding observed with triple (NER) and quadruple (NERE) changes (FIG. 22D). These increases in binding affinity correlated with neutralization potency against isolates that are difficult to neutralize by VRC26.25 (FIG. 16B). These VRC26.25 variants were assayed against a global panel of 13 pseudoviruses. Variants bearing the NER and NERE mutations neutralized these viruses with a geometric mean IC₅₀ more than 6-fold lower than the IC₅₀ of this already very potent antibody (FIG. 16C). It can be concluded that HCDR3-edited cells affinity mature in response to SOSIP immunization. It is further noted that mutations that emerge in multiple mice, and the frequency of those increases with immunization, are likely to enhance neutralization potency.

### HCDR3-Edited B Cells Facilitate Evaluation of SOSIP Antigens.

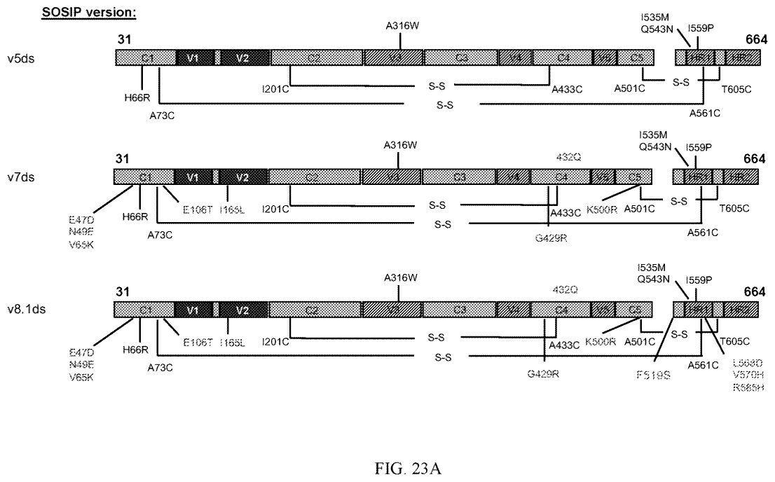

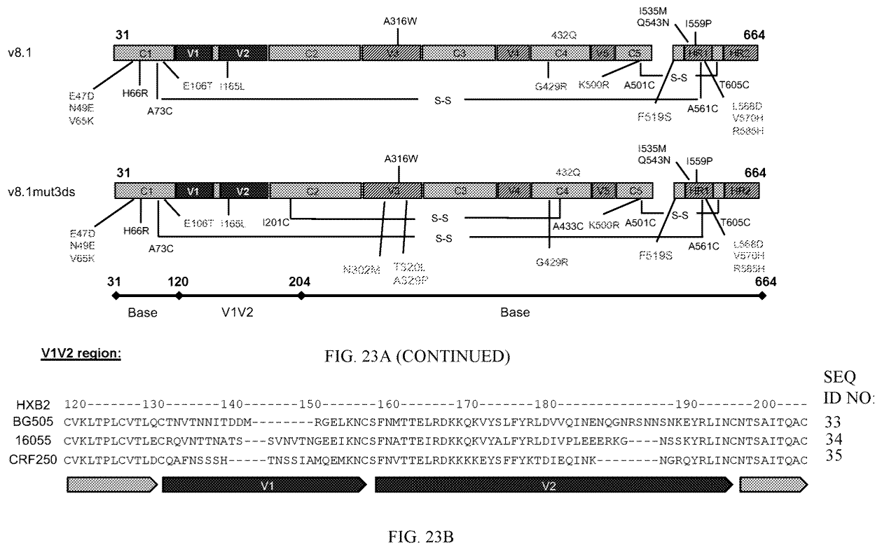

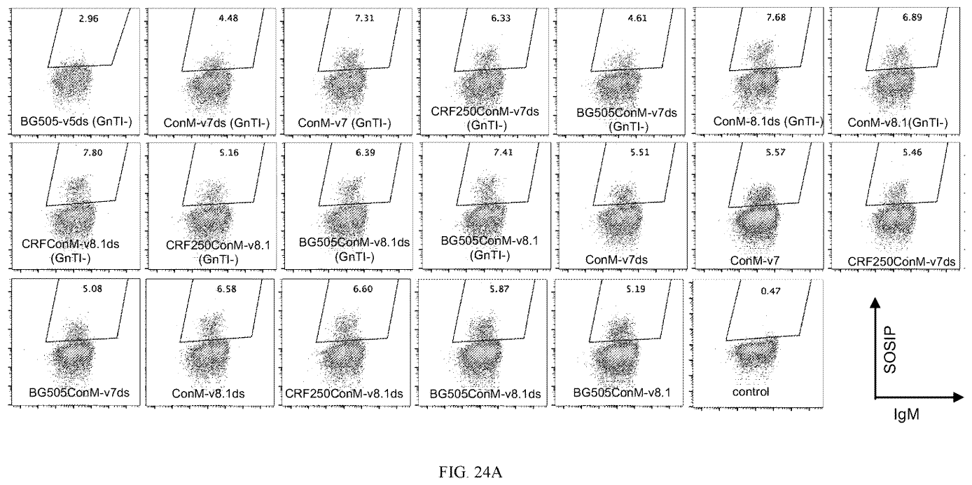

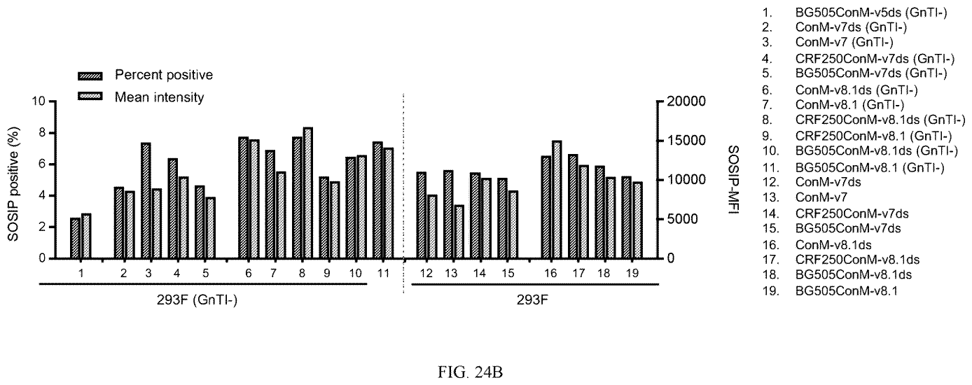

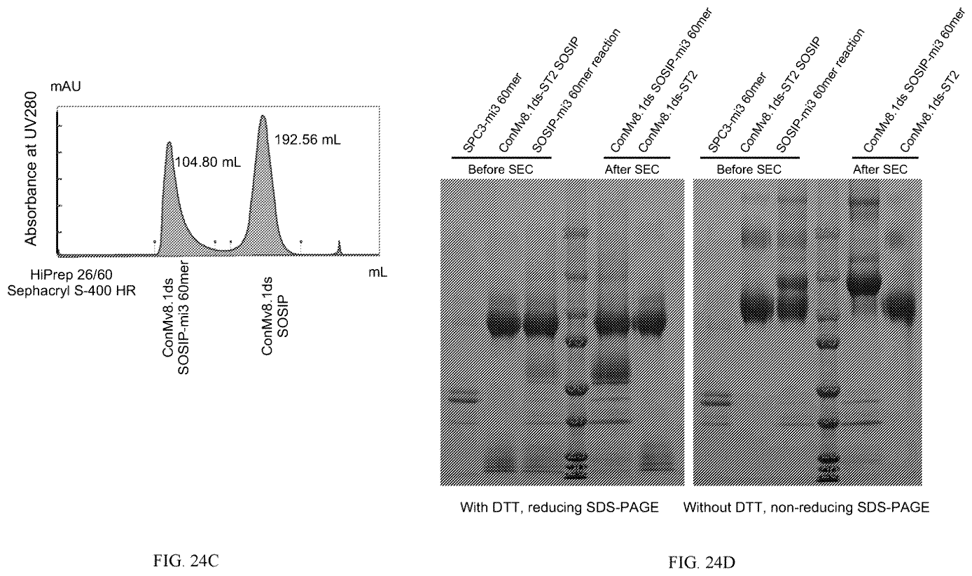

It was then evaluated whether HCDR3-edited B cells could be used to evaluate candidate Env antigens in vivo. A number of SOSIP variants, differing in source isolate, producing cell line, and SOSIP version, were generated (82, 105). These constructs are named according to the source isolate of the V1V2 region (BG505, CRF250, ConM. 16055), the source isolate of the SOSIP base (BG505, CRF250, or ConM), the SOSIP version (v5, v7, v8.1) (65, 81, 82, 106), and whether an additional disulfide loop (I201C-A433C; “ds”) (70, 73, 107), or additional V3-loop mutations, “mut3” (108), are present. Note that SOSIP versions indicate different stabilizing mutations (FIG. 23A) in the base, which is defined as the entire SOSIP trimer subtracting the VIV2 region shown in FIG. 23B. It was observed that SOSIP proteins with v8.1 mutations, and those produced in N-acetylglucosaminyltranferase I (GnTI)-negative cells and thus lacking complex glycans (69, 109), more efficiently bound VRC26.25-HCDR3-edited primary murine B cells (FIG. 24A-B).

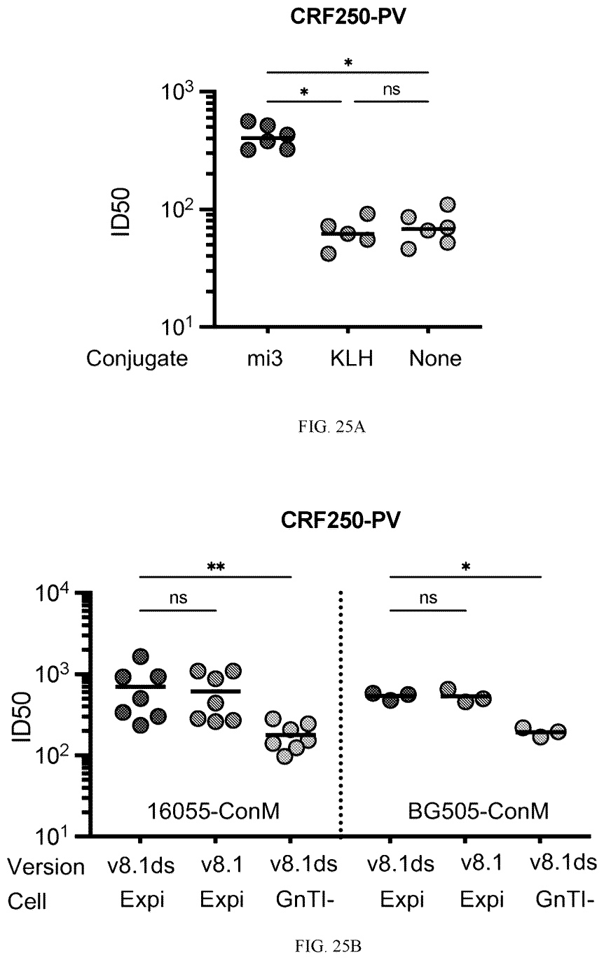

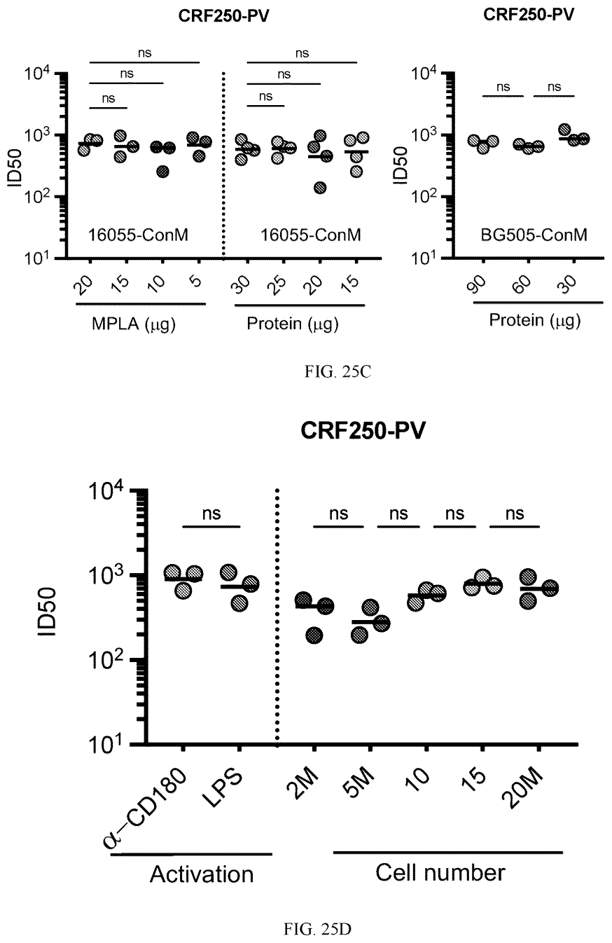

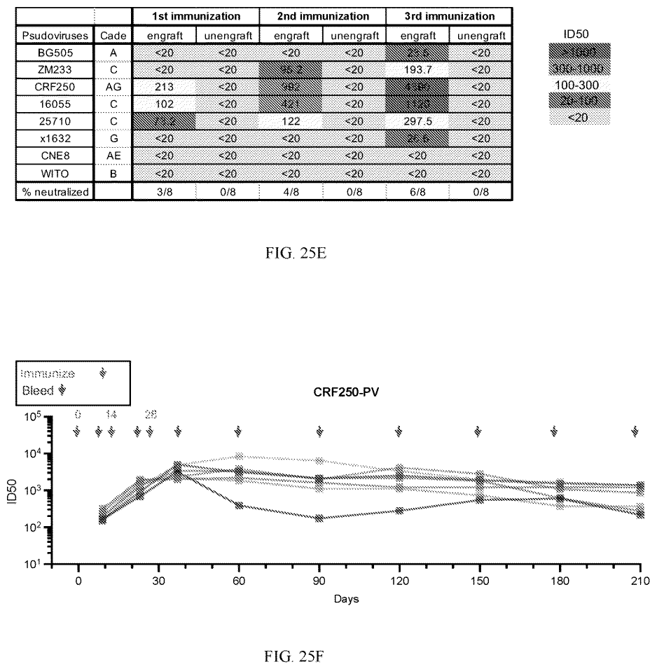

To compare these antigens in vivo, mice engrafted with VRC26.25 HCDR3-edited B cells were immunized up to three times and their sera were harvested one week after each immunization. First, three ways to present the ConM-v8.1-ds SOSIP antigen were compared. Specifically, this SOSIP was either conjugated to a KLH carrier, covalently linked via a SpyTag to the Spycatcher-mi3 60-mer (102, 103), or introduced as a free SOSIP trimer (FIG. 24C-D), each in an MPLA/QuilA mixed adjuvant. Significantly higher neutralizing responses against CRF250 pseudoviruses with the mi3-conjugated SOSIP were observed compared to free SOSIP trimer or KLH-conjugated SOSIP (FIG. 25A). Thus, all further study of protein antigens used mi3-conjugated SOSIP variants. 16055-ConM-v8.1ds and BG505-ConM-v8.1ds SOSIP trimers produced in Expi239F cells or in GnTI-negative Expi293F cells were also compared. It was observed that this SOSIP produced in GnTI-negative cells bound primary HCDR3-edited cells more efficiently (FIG. 24A-B) but elicited significantly less potent neutralizing sera (FIG. 25B). A series of other conditions were compared and it was observed that neutralizing responses were relatively independent of the amount of adjuvant or antigen (FIG. 25C). Similarly, the method of ex vivo activation (LPS or anti-CD180), or the number of adoptively transferred donor cells did not significantly affect neutralization (FIG. 25D). Accordingly, in subsequent experiments, LPS was used to activate B cells ex vivo, and 25 mg SOSIP-mi3 multimers adjuvanted with 20 mg MPLA was used for immunizations. In most cases, around 10 million cells, were adoptively transferred to a mouse. Since 3-4% of these transferred cells express the VRC26.25 HCDR3 (FIG. 14C; FIG. 21C), engineered cells initially comprise less than 0.4% of the host mouse B cells.

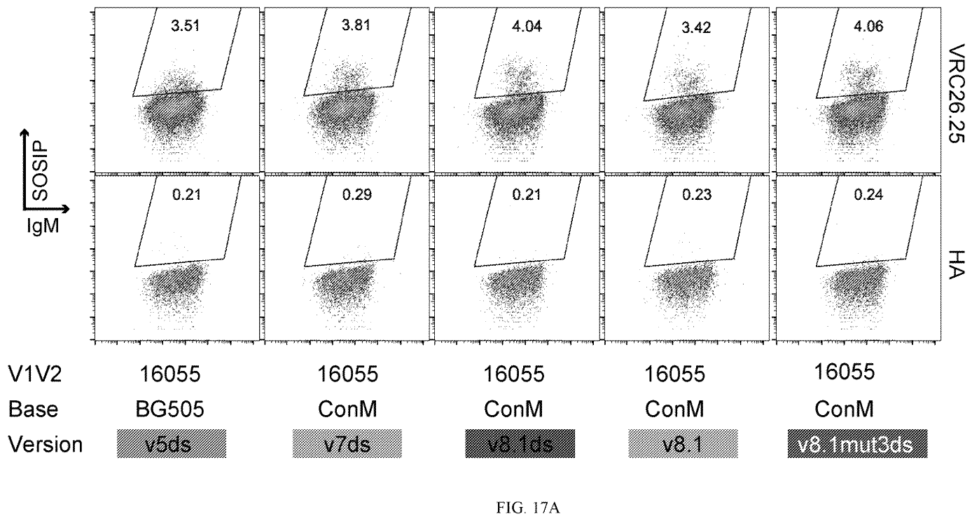

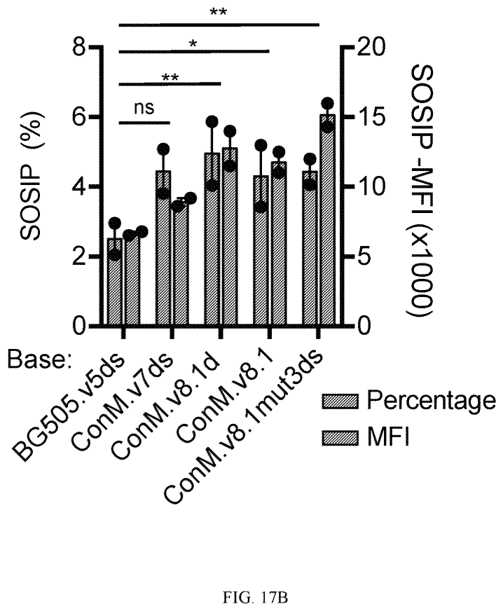

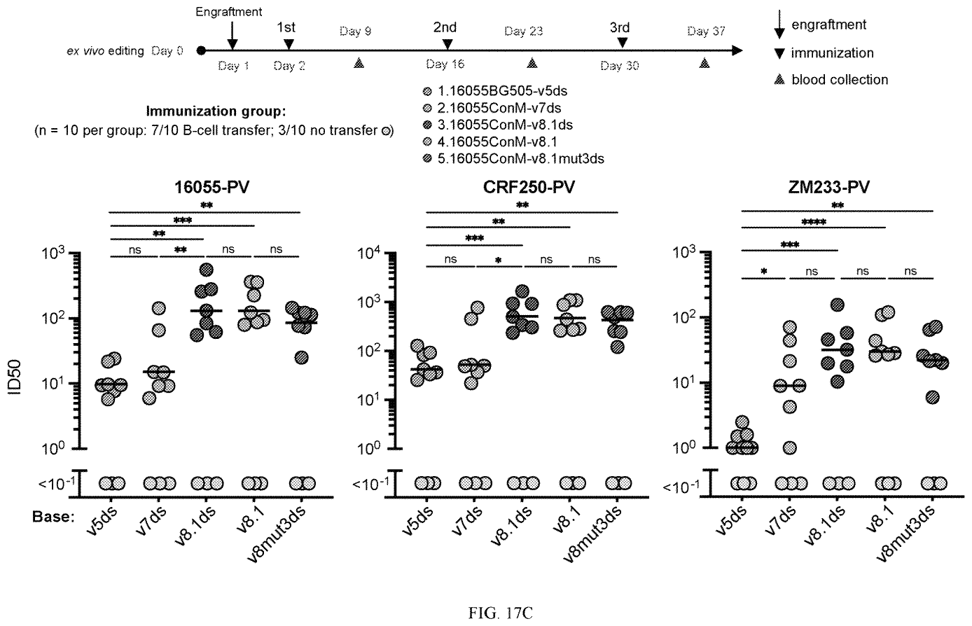

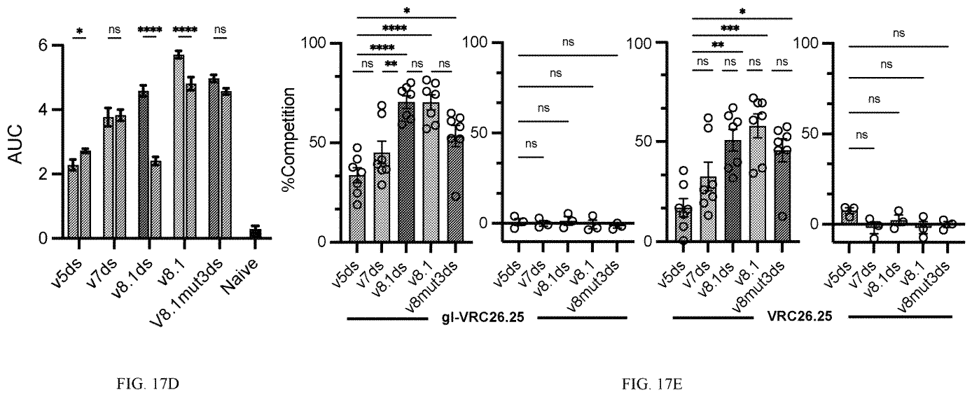

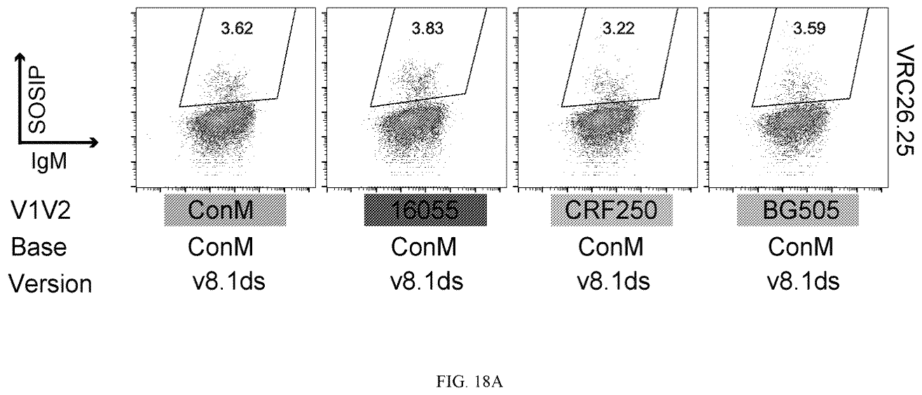

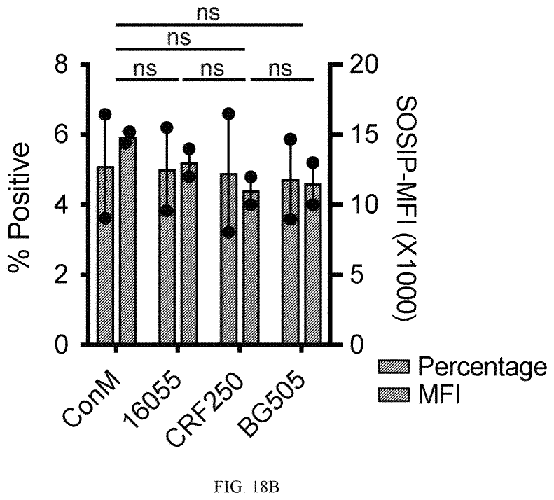

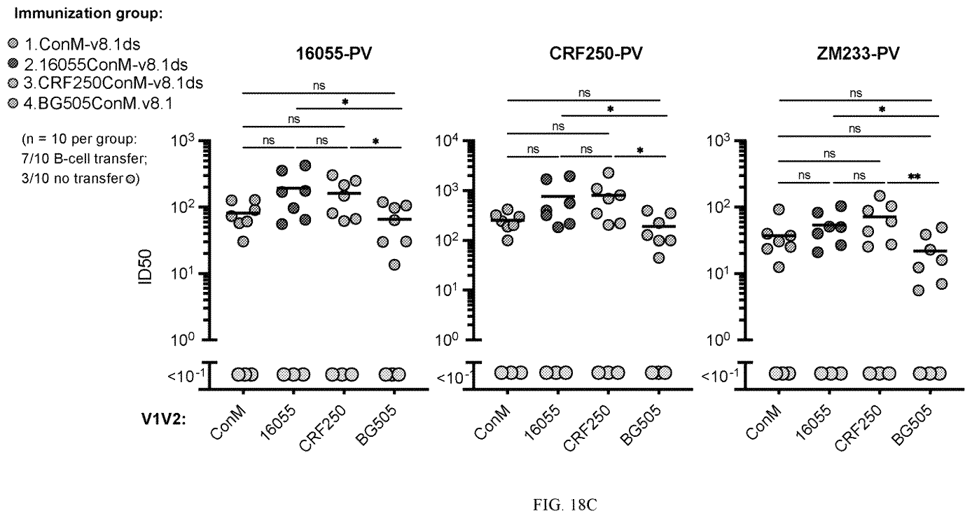

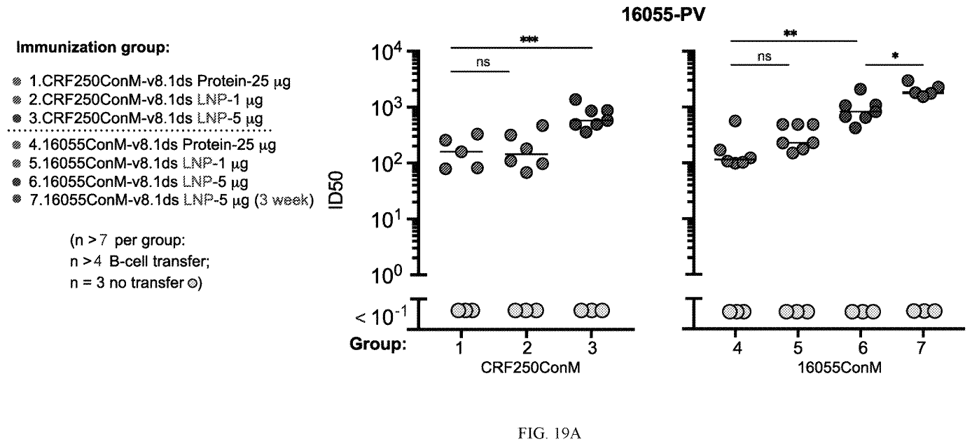

A number of SOSIP variants were then generated, each bearing the VIV2 region of the 16055 isolate with a BG505 or ConM base, and directly compared their ability to bind VRC26.25 HCDR3-edited primary murine cells (FIG. 17A-B). The ConM base was focused on because it is designed to broaden the antibody responses with the absence of immunodominant holes in the glycan shield³⁸ and rare, isolate-specific residues (82). It was observed that constructs based on a previously reported v8.1 SOSIP platform bound edited B cells slightly stronger than those based on earlier designs. Further, mice adoptively transferred with edited cells and immunized with v8.1 SOSIPs raised more potent neutralizing responses against all three isolates tested (FIG. 17C). Again, sera from mice that did not receive edited cells did not neutralize any isolate (gray dots), even though anti-SOSIP antibodies could be detected by ELISA in these sera (FIG. 17D). Notably, one SOSIP, 16055-ConM-v8.1ds, elicited significantly more binding antibodies in VRC26.25-HCDR3 engrafted mice than in unengrafted mice vaccinated in the same manner, suggesting that this SOSIP raised fewer non-neutralizing murine antibodies (compare grey to colored bars in FIG. 17D). Differences in neutralization potency correlated with the ability of these sera to compete with VRC26.25 or its inferred germline precursor for binding to the 16055-ConM-v8.1ds SOSIP (FIG. 17E). It is concluded that SOSIP variants differ in their ability to bind VRC26.25 HCDR3-edited B cells even when their V1V2 regions are identical, and that these variants also differ in their ability to focus immune responses to the V1V2 region. It was further observed that SOSIP proteins based on the v8.1ds platform with a ConM base and a 16055 V1V2 region elicited potent apex-focused neutralizing responses. Sera elicited by this SOSIP variant included heterologous neutralizing responses that broadened with each immunization and persisted for more than 200 days (FIG. 25E-F). The ability of different V1V2 regions to elicit apex-antibodies were also compared. V1V2 regions of four VRC26.25-sensitive isolates were compared on a ConM-v8.1ds base. Each bound HCDR3-edited B cells with similar efficiencies (FIG. 18A-B), but the SOSIP protein expressing the V1V2 region of BG505 generated less potent neutralizing responses against three pseudoviruses (FIG. 18C). Interestingly, these differences were less pronounced than those associated with different SOSIP bases, suggesting that the frequency with which a SOSIP populates a closed conformation is more important than the sequence and structure of these four V1V2 domains. Collectively, these data suggest that HCDR3-edited B cells can provide key insight into the in vivo properties of Env antigens.

SOSIP-TM Proteins Expressed from mRNA Vaccines Raise More Potent Neutralizing Responses Than Multimeric Protein Antigens.

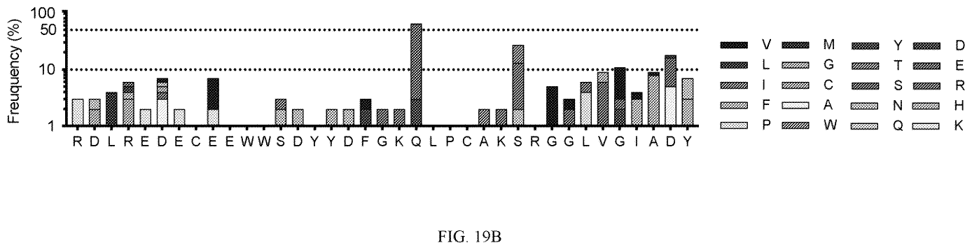

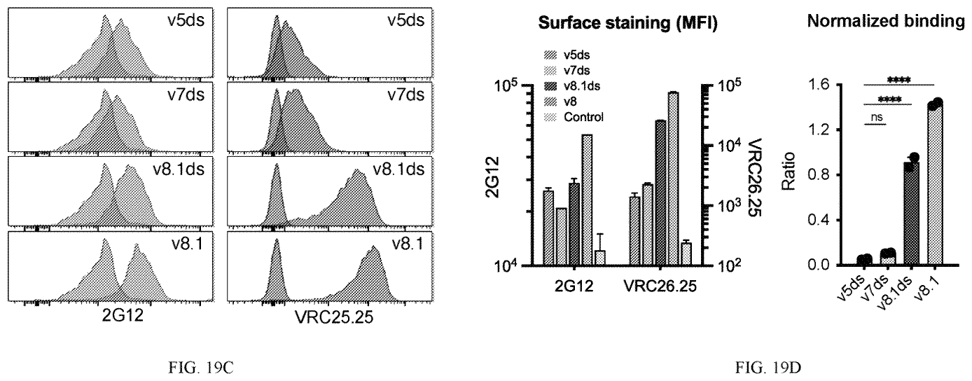

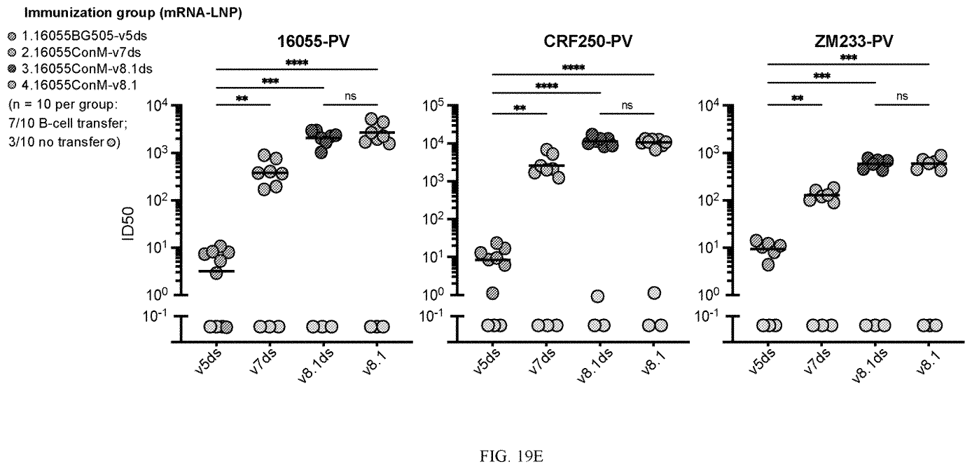

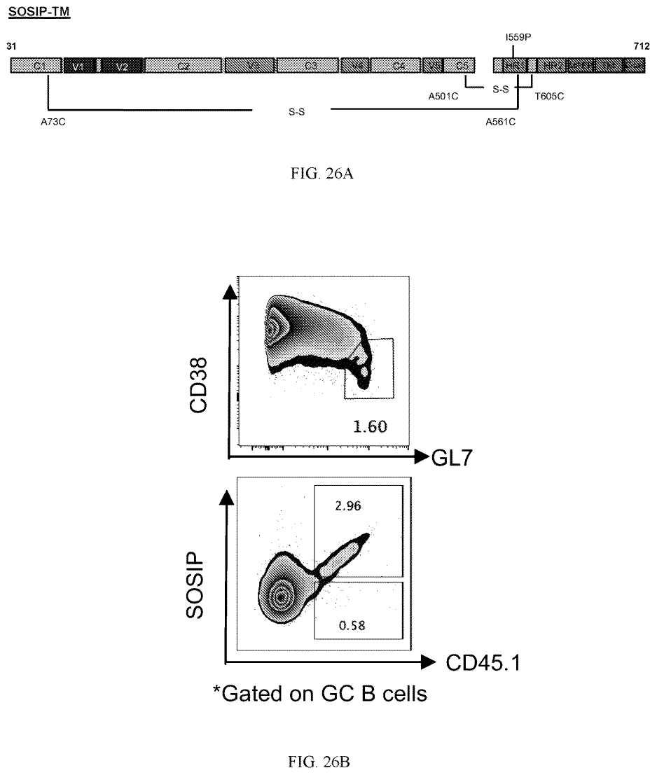

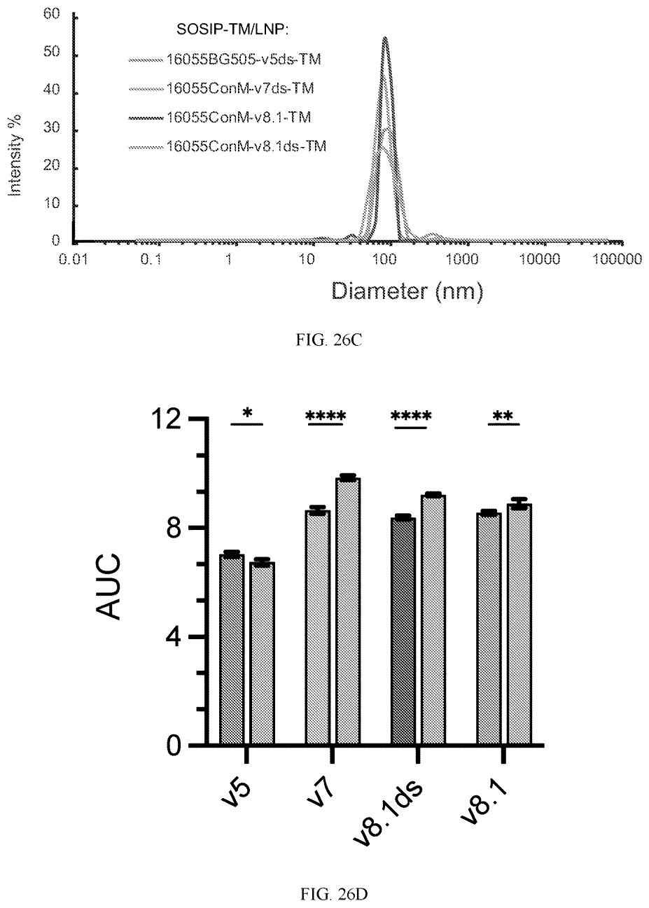

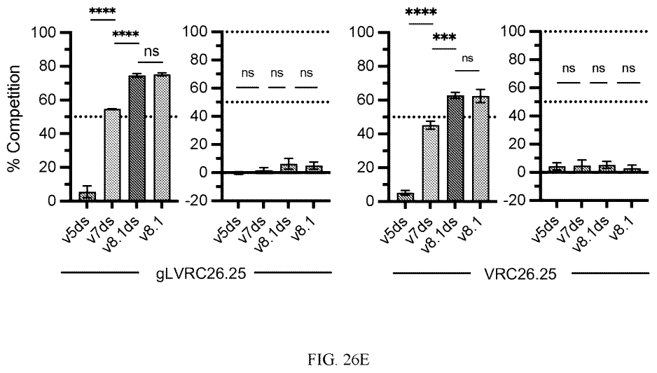

Using this mouse model, the immunogenicity of adjuvanted ConM-based SOSIP mi3-multimers was compared to an mRNA-LNP-delivered immunogen expressing the same SOSIP extended through Env residue 712 (HXB2 numbering), thus including the ConM transmembrane (TM) domain and a truncated cytoplasmic region (SOSIP-TM. FIG. 26A). Again. VRC26.25 HCDR3-edited B cells were engrafted into recipient mice, which were then immunized with protein SOSIP mi3-multimers or mRNA-LNP expressed SOSIP-TM. In both cases, ConM-v8.1ds SOSIP/SOSIP-TM antigens bearing either the CRF250 or 16055 VIV2 regions were evaluated (FIG. 19A). Sera collected 7 days after the second immunization were characterized for neutralization activity against 16055 pseudoviruses. It was observed that a 1 μg dose of SOSIP-TM delivered as mRNA-LNP raised comparable neutralizing responses to 25 μg of SOSIP mi3-multimers, whereas 5 μg of SOSIP-TM mRNA-LNP elicited a significantly more potent neutralizing response. It should be noted that 25 μg of adjuvanted protein antigen is a high dose for murine studies (181, 182), whereas 5 μg is lower than previous studies of mRNA-expressed viral antigens (94, 112, 113). The most efficient neutralizing response was observed when mice were boosted at 3-week intervals (FIG. 19A), and B cells from these mice were further analyzed. It was observed that mRNA-LNP immunization induced strong germinal-center reactions (FIG. 26B) and robust SHM (FIG. 19B). The pattern and rate of SHM was broadly similar to protein immunization with SOSIP mi3-multimers observed in FIG. 16A (FIG. 19B). mRNA-LNP expressing four SOSIP-TM variants corresponding to the SOSIP variants characterized in FIG. 17C were further generated. LNP were quality controlled through dynamic light scattering (FIG. 26C) and through cell-surface expression of SOSIP-TM on 293T cells, as measured by the antibodies 2G12 and VRC26.25 (FIG. 19C-D). SOSIP-TM variants expressed comparably as indicated by 2G12 binding but varied in their relative binding to VRC26.25, with v8.1 and v8.1ds binding this apex bnAb most efficiently. These same mRNA-LNP were then compared for their ability to raise anti-apex responses in vivo. As with proteins antigens, v8.1 and v8.1ds SOSIP-TM variants most efficiently raised apex-specific neutralizing responses (FIG. 19E). Sera from the unengrafted mice bound SOSIP trimers comparably to engrafted mice as indicated by ELISA (FIG. 26D), but only engrafted mice raised anti-apex responses and these responses correlated with the neutralization activities of these sera (FIG. 26E). These data indicate that the advantages of ConM-v8.1 and ConM-v8.1ds persist across platforms and modes of presentation.

mRNA-LNP Elicit Neutralizing Sera in Mice Engrafted with B-Cells Engineered to Express the HCDR3s of PG9. PG16, or the VRC26-UCA.

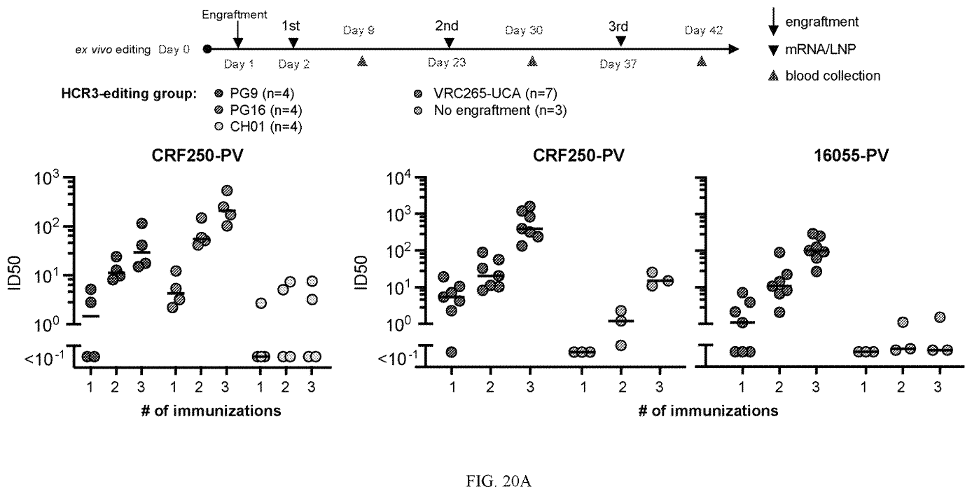

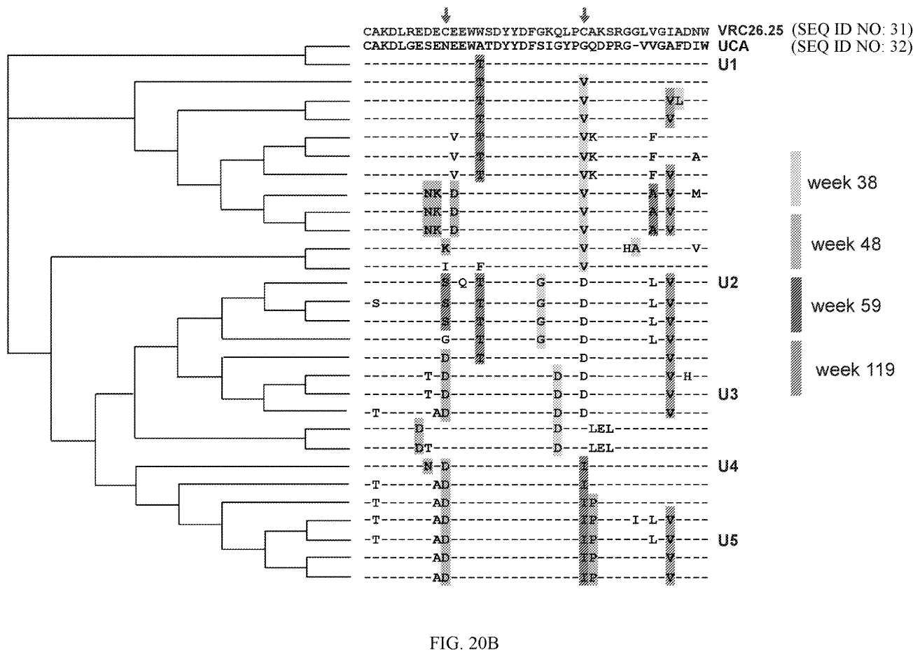

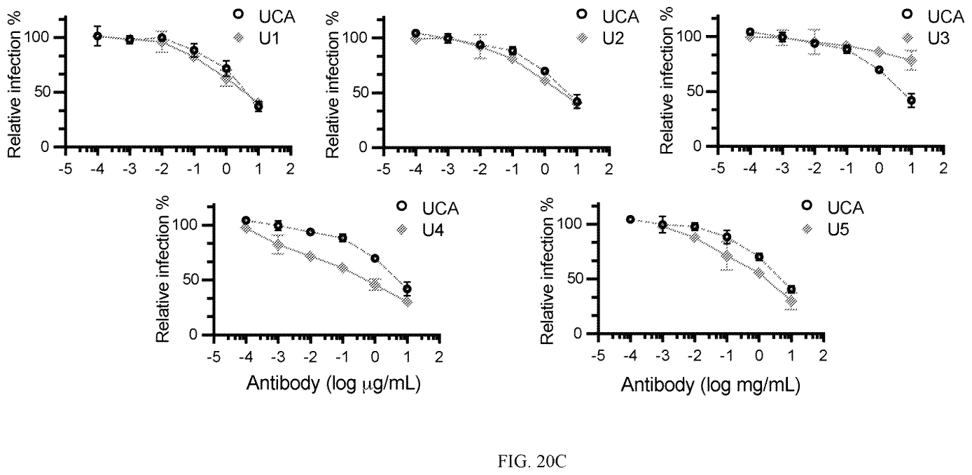

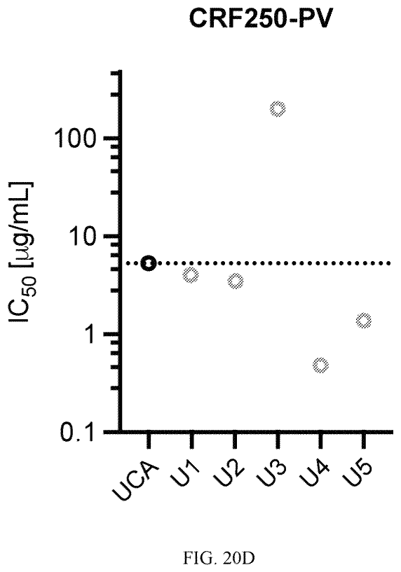

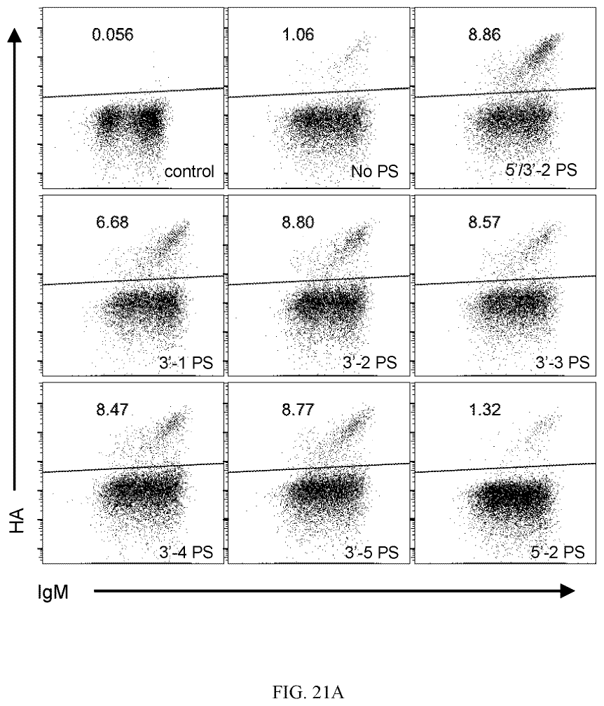

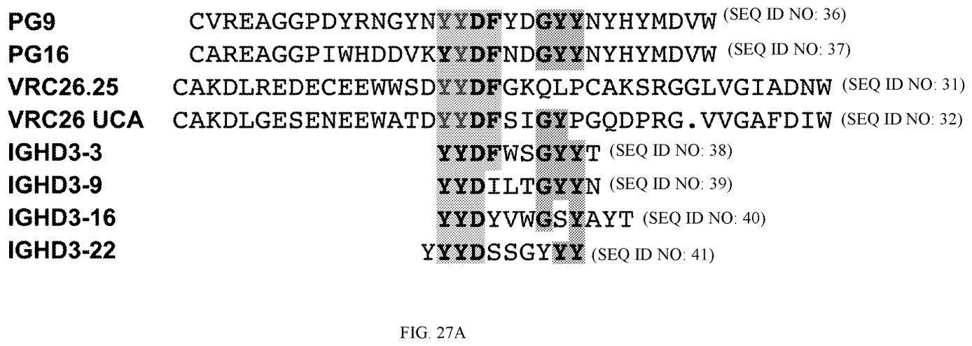

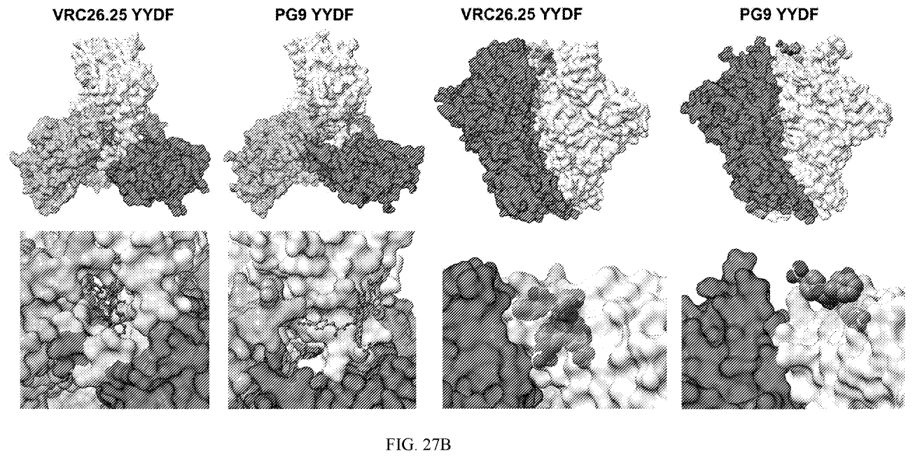

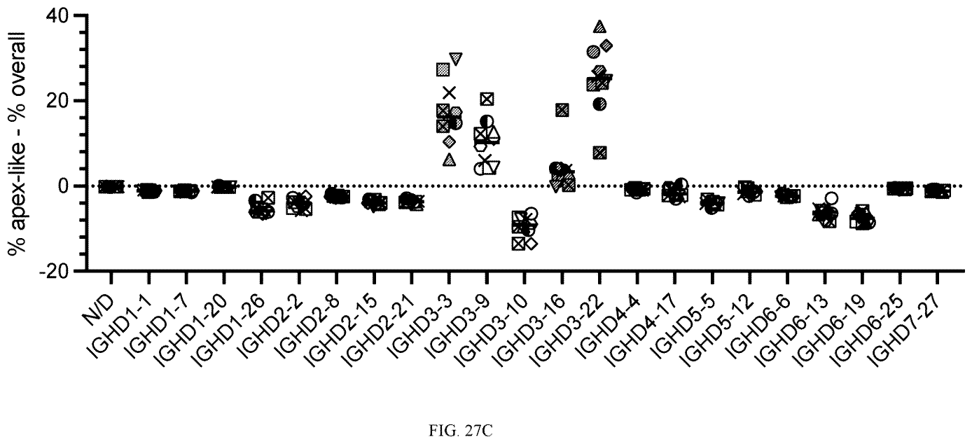

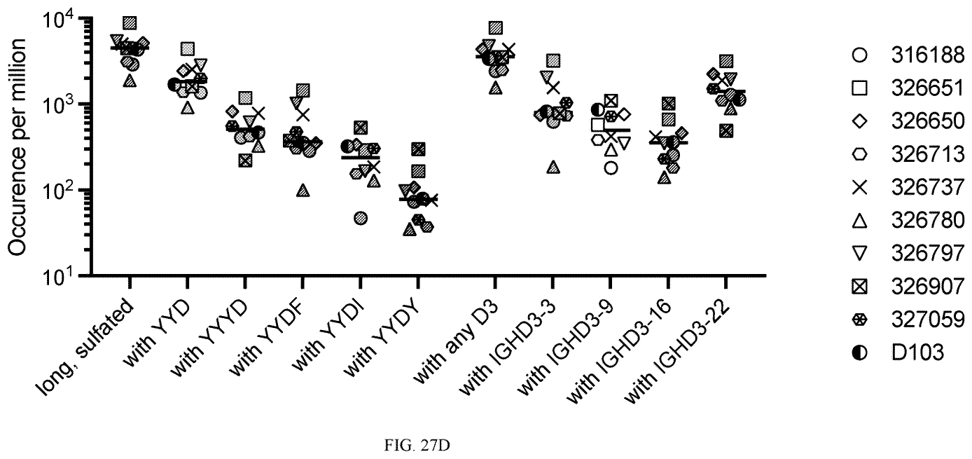

All previous experiments used the mature VRC26.25 HCDR3. To determine if a wider range of apex-targeting HCDR3s could also respond to SOSIP-TM immunization, B cells were engrafted with the HCDR3s of the apex bnAbs PG9, PG16, and CH01, as well as the VRC26-UCA. Mice were immunized with mRNA-LNP encoding SOSIP-TM variants of 16055-ConM-v8.1 or CRF250-ConM-v8.1ds. Neutralization responses against the CRF250 or 16055 pseudoviruses were observed after three immunizations in mice engrafted with the PG9, PG16, and the VRC26-UCA HCDR3 defined in (61) (FIG. 20A). No responses were observed using the HCDR3 of CH01, consistent with its lower affinity and greater dependence on non-HCDR3 elements. Notably, PG9, PG16, VRC26.25, and the VRC26-UCA, but not CH01, share a common tyrosine-sulfated YYDF motif (SEQ ID NO: 42) derived from the D3-3 gene segment (FIG. 27A). The maturation of cells edited to express the VRC26-UCA HCDR3 as defined by Doria-Rose et al (62), was further analyzed by NGS. A phylogenetic tree was constructed from the most frequently observed sequences in mice engrafted with B cells expressing this HCDR3 (FIG. 20B). It was observed that these affinity-matured UCA HCDR3 acquired multiple mutations also found in the VRC26 variants isolated from the original donor, CAP256 (61, 114). As in that donor, most active hypermutations focused on residues near a disulfide bond found in more mature VRC26-lineage members. The five most frequently observed HCDR3s were characterized for their ability to neutralize CRF250 pseudoviruses (FIG. 20C-D). Four of these five improved the potency of the original VRC26-UCA HCDR3, indicating affinity maturation of this UCA. Thus. SOSIP-TM-expressing mRNA-LNP-delivered immunogens can elicit measurable immune responses in mice engrafted with cells expressing the HCDR3 of multiple apex bnAbs. In doing so, they enable quantitative comparison of candidate HIV-1 vaccines and help to define HCDR3 motifs likely to bind these immunogens.

### Toward an HCDR3-Focused Strategy for HIV-1 Vaccine Development.

Among the HCDR3 motifs (FIG. 27A), the tyrosine-sulfated YYDF sequence (SEQ ID NO: 42) derived from D3-3 appears especially important because it is the common motif shared by HCDR3s elicited by SOSIP-TM and because these residues directly engage the Env apex at two sites and in two orientations (FIG. 27B), both contributing substantially to Env binding (54, 115). To determine the frequency of potential apex bnAb precursors, long HCDR3s with candidate sulfation motifs from 10 previously characterized human donors were analyzed (57). This analysis defines a potential apex precursor as an antibody with HCDR3 that are 24 amino acids or longer, with a tyrosine-sulfation motif that is 7 residues or more from the beginning of the HCDR3 and 10 amino acids or more from its end. A tyrosine-sulfation motif is broadly defined as at least one tyrosine adjacent to one acidic amino acid, without an adjacent basic residue (116-119). A significant enrichment of candidate precursors for HCDR3 deriving from D3-3, D3-9, D3-16, and D3-22 (FIG. 27C) was observed, likely due to the longer lengths of these D gene segments and the presence of sulfation motifs in their dominant reading frames. FIG. 27D shows the frequency of these diversity chains and of the specific YYDX sequences encoded by them. An average of one in approximately 200 HCDR3 met these length and sulfation criteria. One in 1800 meet these criteria and encode YYDF (SEQ ID NO: 42). These numbers suggest a considerably higher precursor frequency than for VRC01-class bnAbs, estimated to be 1 in 2.4 million (120). The mouse model described here helps iteratively refine this definition of apex bnAb precursor and help identify SOSIP-TM variants that best prime and affinity mature these precursors.

### DISCUSSION

A new murine model that can facilitate in vivo evaluation of trimeric Env antigens and HIV-1 vaccination strategies is described herein, especially those designed to elicit apex bnAbs. This model relies on two distinctive properties of apex bnAbs. First, these bnAbs only bind native-like trimers in a closed conformation. This closed structure occludes immunodominant non-neutralizing epitopes and presents conserved neutralizing epitopes as they are presented by functional Envs on the virion. This model thus provides quantitative insight into the in vivo stability and immunogenicity of antigens designed to maintain this structure. A second property of several apex bnAbs, highlighted by the data here, is their near complete reliance on their long HCDR3s to bind Env, providing sufficient affinity to elicit a neutralizing response from diverse murine B cells expressing these HCDR3s. The resulting model is adaptable, sensitive, and efficient, as indicated by its ability to affinity mature the HCDR3s of the bnAb VRC26.25 and its UCA.

This system has a number of advantages over other animal models of vaccination. First, in contrast to previous approaches using transgenic mice,^{9,10,79,80} it can be established or modified in days, greatly accelerating the developmental cycle of SOSIP and SOSIP-TM antigens. Second, this model enables selection among combinations or libraries of HCDR3s that would be difficult using transgenic mice. Third, in contrast to other murine B-cell editing approaches,^{53,54,56,57} this approach does not disrupt the underlying regulatory apparatus at the B-cell locus. Rather, this approach directly overwrites the native HCDR3 without displacing the heavy and light-chain genes or introducing exogenous regulatory elements. Perhaps as a consequence, robust somatic hypermutation and clear affinity maturation of the exogenous HCDR3, not reported with other systems, were observed,^{53,55-57} Fourth, in contrast to most B-cell editing strategies, the underlying BCR diversity is maximized. This underlying diversity better emulates a human repertoire, especially in HIV-1-negative persons, and its preservation has proved valuable in other systems, most notably with transgenic mice expressing human-derived variable chains to evaluate antigens designed to elicit CD4-binding site antibodies.⁹ Fifth, in contrast to wild-type animal models like mice, rabbits, or primates, this system presents human HCDR3s. This property is especially critical because the D gene segments that help form these HCDR3s are highly species specific.^{49,50} For example, no non-human species has a D segment homologous to human D3-3, D3-9, D3-16, or D3-22. These D segments are relatively long and directly encode sulfation motifs. Thus in vivo testing of antigens designed to elicit human HCDR3 would ideally be performed directly in humans or in the system presented here.

Using this system, several insights useful for generating an apex-targeting vaccine were gleaned. First, it was observed that design differences among SOSIP immunogens significantly altered their ability to elicit apex-focused antibody responses. For example, multiple SOSIP proteins with identical V1V2 and therefore apex regions, but with different stabilizing mutations were compared. It was observed that those bearing v8.1 mutations were significantly more immunogenic. Notably, the ability of these SOSIP variants to bind HCDR3-edited murine B cells did not fully predict their immunogenicity, underscoring the necessity of in vivo models that anticipate human immune responses. It was also observed that v8.1 SOSIP variants elicited a less pronounced non-neutralizing response in unengrafted mice. Thus, this closed-form trimer may more effectively occlude non-neutralizing decoy epitopes. Second, the data presented herein makes clear that mRNA-delivered vaccine candidates expressing these SOSIPs with a transmembrane domain were significantly more immunogenic than soluble SOSIP multimers administered as an adjuvanted protein. Notably. SOSIP variants that elicited more potent responses when presented by mi3-conjugated multimers also elicited stronger responses when expressed as SOSIP-TM and delivered by mRNA-LNP, suggesting that stability improvements useful for soluble SOSIP antigens extend to their use in mRNA vaccines. Third, the data show that SOSIP-TM can elicit neutralizing antibodies from B cells edited to express four distinct HCDR3s, those of PG9. PG16, VRC26.25, and the predicted VRC26 progenitor. Thus, the problem of developing a useful-if not comprehensive-vaccine might be reduced to the more tractable goal of eliciting long, sulfated HCDR3 that bind the apices of multiple isolates. Fourth, in contrast to the CH01 HCDR3 which did not generate a neutralizing response in engrafted mice, all HCDR3 that elicited such a response share a sulfated D3-3-encoded YYDF motif (SEQ ID NO: 42). The HCDR3-edited mouse model described here allows the testing of the hypothesis that SOSIPs that most effectively drive maturation of D3-3-expressing HCDR3s, or those deriving from related D segments bearing YYD, can raise apex-binding bnAbs in uninfected humans. As importantly, this model allows us to refine the definition of an apex bnAb precursor by testing additional HCDR3 or HCDR3 libraries derived from human repertoires.

Finally, the data show that repeat immunizations with the same native-like SOSIP-TM immunogen can elicit a neutralizing response against circulating Envs from a bnAb UCA. These responses were generated with an initial frequency of 0.2 million transferred UCA-expressing precursor B cells (approximately 0.2% of B cells in a mouse), similar to the frequency of long potentially sulfated HCDR3 observed in humans (0.45%), and to the subset of these HCDR3 containing YYD (0.21%). Together these observations suggest that SOSIP-TM antigens have the potential to engage these candidate apex precursors to generate a neutralizing response in humans.

In summary, a useful and adaptable system that can accelerate evaluation and development of candidate antigens for an HIV-1 vaccine has been developed. It is also shown that mRNA-expressed SOSIP-TM can generate neutralizing antibodies from B cells edited to express four divergent HCDR3, raising the possibility that BCR with similar HCDR3s can be matured to apex bnAbs in uninfected humans.

### EXPERIMENTAL MODEL AND SUBJECT DETAILS

### Mice

Nine to 12 week-old CD45.1-positive mice (B6.SJL-Ptprc^{a} Pepc^{b}/BoyJ, strain 002014) from Jackson Laboratories were used as a source of splenic B cells. Age- and gender-matched CD45.2-positive C57BL/6J strain mice (Jackson Laboratories, strain 000664) mice were used as host mice for B cell transplantation and immunizations.

All mice were housed and cared at the institutional animal facility in UF Scripps (Jupiter, FL), following the Animal Welfare Act and other federal, state, and local policies and regulations. All animal experiments were reviewed and approved by the Institutional Animal Care and Use Committee under the approval protocol number 21-010-01.

### METHOD DETAILS

### Mouse Splenic B Cell Activation and Electroporation

Whole spleens from 9-12 week old CD45.1-positive donor mice were pulverized and mechanically crushed on the inner top of 70 μm cell strainers in RPMI 1640 medium (Thermo Fisher Scientific, 61870127) with 2% FBS (Thermo Fisher Scientific, 26140-079). After red blood cell lysis in a NH₄Cl solution (BD Biosciences, 555899) at room temperature for 3 minutes, B cells were neutralized with Ca²⁺/Mg²⁺ free PBS with 0.5% BSA (Sigma-Aldrich, A1470) and 5 mM EDTA and then isolated using mouse B cell purification kit (Miltenyi Biotec, 130-090-862) and LS columns (Miltenyi Biotec, 130-042-401). Before electroporation, B cells were activated for 36-42 hours in RPMI 1640 medium with 10% FBS, 100 μM Non-Essential Amino Acids (NEAA. Thermo Fisher Scientific, 11140050). 1 mM sodium pyruvate (Thermo Fisher Scientific, 11360070), 10 mM HEPES (Thermo Fisher Scientific, 15630080), 55 μM 2-Mercaptoethanol (Thermo Fisher Scientific, 21985023), 100 units/mL penicillin and 100 μg/mL streptomycin (Thermo Fisher Scientific, 15140163), and either (1) 4 μg/ml anti-mouse CD180 antibody (BD Biosciences, 562191), (2) 50 μg/ml LPS (Sigma-Aldrich. L2880), or (3) 10 μg/ml LPS and 10 ng/ml mouse IL-4 (PeproTech Inc. 214-14).

After activation, B cells were harvested and washed twice with Ca²⁺/Mg²⁺ free PBS at room temperature. For each 100 μl of electroporation reaction using the nucleocuvette vessels (Lonza, V4XP-4024), approximately 5 million cells were suspended in 74 μl of P4 Primary Cell solution (Lonza, VSOP-4096). In parallel, 3.12 μl of PBS, 1.26 μl of 1M NaCl, 1.12 μl of 250 μM Mb2Cas12a (produced in house),⁵⁹ and 4.5 μl of 100 μM gRNA (5′-UAAUUUCUACUGUUUGUAGAUCUUGACCCCAGUAGUCCAUAGCA-3′) (SEQ ID NO: 43) were mixed and incubated at room temperature for 15 min for the RNP complex formation. These 10 μl of RNP were then incubated with 16 μl of 100 μM single strand DNA donor for 3 minutes at room temperature. The above 26 μl mixture was then mixed with the 74 μl of suspended B cells and transferred to the nucleocuvette vessels for electroporation in the Lonza 4D nucleofector under the DI-100 program. For larger scale electroporation in the 1 ml scale of Nucleocuvette Cartridge (Lonza, V4LN-7002), reactions were scaled up 10 fold for cell suspension, RNP, and donors. After electroporation, cells were rested for 10 minutes in nucleocuvette vessels or cartridges and then transferred to preheated activation medium without penicillin-streptomycin or LPS, which were added one hour later.

### Mouse B Cell Transplantation

Approximately 18 hours after electroporation. B cells were washed with prechilled Ca²⁺/Mg²⁺ free PBS for four (for LPS activated cells) or three times (for anti-mCD180 antibody activated cells) and then suspended in prechilled 5% horse serum (Cytiva, SH3007403HI) solution in PBS with Ca²⁺/Mg²⁺ (Thermo Fisher Scientific. 14040133). After filtration (Falcon. 352235) and counting, the indicated number of cells for each mouse were transplanted in a 100 μl volume via retro-orbital injection under anesthesia with isoflurane. An aliquot of approximately 2 million cells were further cultivated in RPMI 1640 medium with 10% FBS, 100 μM NEAA, 1 mM sodium pyruvate, 55 μM 2-mercaptoethanol, 10 mM HEPES, 100 units/mL penicillin and 100 μg/mL streptomycin, 5 μg/ml LPS, 10 ng/ml mouse IL-4, and 2 μg/ml anti-mouse CD180 antibody for additional 36-48 hours to monitor editing efficiency validation by flow cytometry.

### Protein Production, Purification, and Conjugation

Protein expression plasmids were constructed using dsDNA gBlocks from IDT and NEBuilder HiFi DNA Assembly Cloning Kit (NEB, E5520S) and then transformed in NEB 5-alpha competent cells (NEB, C2987U). Expi293F (Thermo Fisher Scientific, A14527) and GnTI-Expi293F (Thermo Fisher Scientific. A39240) cells were maintained in Expi293TM Expression Medium (Thermo Fisher Scientific. A1435102) following the manufacturer's instructions. Cells were diluted to three million/ml in fresh and preheated medium (Thermo Fisher Scientific, A1435102), and then transfected with FectoPRO reagent (Polyplus, 116-040). To produce SOSIP proteins, plasmids expressing the indicated SOSIP protein, furin, and PDI (protein disulfide-isomerase) were co-transfected at 4:1:1 ratio. For antibody production, plasmids expressing the antibody heavy and light chains were co-transfected at 1:1.25 ratio. In the case of the tyrosine-sulfated PGT145 and VRC26-family antibodies, heavy chain, light chain, and TPST2-expressing plasmids were co-transfected at the ratio for 1.78:2.2:1. For Spc3-mi3-ctag production, Spc3-mi3-Ctag- and PDI-expressing plasmids were co-transfected at the ratio of 4:1. Four to 5 days post-transfection, cell supernatants were harvested, centrifuged, and filtered before purification.

PGT145 and VRC26-family antibodies were captured with Protein A columns (Cytiva, 11003493) and gently eluted with 3 M MgCl₂ solution (Thermo Fisher Scientific, 21027). Elutions were then buffer exchanged to HEPES buffer (10 mM HEPES pH 8.0 and 75 mM NaCl) firstly and exchanged to PBS with desalting columns (Thermo Fisher Scientific, 89894) following the manufacturer's instructions. Antibodies were finally concentrated by ultrafiltration. SOSIPs were purified with PGT145 affinity columns and gently eluted with 3 M MgCl₂ solution, after buffer exchange, trimers were purified by SEC (size exclusion) in the Superdex 200 Increase 10/300 GL column (Cytiva, 28990944) or HiPrep 26/60 Sephacryl S400 HR column (Cytiva, 28935605). Spc3-mi3-Ctag 60-mer were purified with anti-Ctag affinity columns followed with gentle elusion and buffer exchange then the 60-mer were purified by SEC in HiPrep 26/60 Sephacryl S400 HR column. SOSIPs were conjugated onto keyhole limpet hemocyanin carrier protein with Imject EDC mcKLH Spin Kit (Thermo Fisher Scientific, 77671) in accordance with the manufacture's protocol.

For conjugation to the mi3 60-mer using the SpyTag/SpyCatcher system, purified SOSIP trimers with C-terminal Spy-tag-2 (ST2) and Spc3-mi3-Ctag 60-mer with N-terminal SpyCacher-3 (Spc3) were conjugated at molar ratio of 2:1. Conjugated SOSIP-mi3 multimers were then purified from the free SOSIP-trimers by SEC in HiPrep 26/60 Sephacryl S400 HR column. Conjugation fractions from SEC were pooled, concentrated, and validated with electrophoresis in native, reducing, and non-reducing denaturing SDS-PAGE gels.

mRNA Lipid Nanoparticle Production

Codon-optimized genes encoding SOSIP variants fused to the Env C-terminal transmembrane domain (TM) sequence were inserted into a pUC vector with 5′ UTR, 3′ UTR, and polyA sequences under T7 promotor. For in vitro transcription (IVT), the DNA templates were linearized by digestion with HindIII and ScaI (NEB) and purified by phenol-chloroform extraction. IVT was then performed using MEGAscript T7 Transcription Kit (Thermo Fisher Scientific, AMB-1334-5) according to the manufacturer's instructions with modifications as using the CleanCap Reagent AG (TriLink, N-7413) and ml-pseudouridine-5′-triphosphate (TriLink, N-1081). Template DNA was digested with Turbo DNase, and synthesized mRNA was purified by LiCl precipitation and 75% ethanol washing. After RNA qualification via electrophoresis in a denaturing agarose gel, double stranded RNA was then removed by cellulose (Sigma-Aldrich, C6288) depletion. The mRNA solution was then precipitated with 3M sodium acetate pH 5.2 and washed with isopropanol and then 75% ethanol. Finally, the RNase free water suspended mRNA were quantified and stored at −80° C. before LNP formulation.

mRNA LNP were formulated via mixing cartridges in the NanoAssembr BenchTop instrument (Precision, NIT0055) according to the manufacturer's instructions. First, mRNA was diluted to 0.1-0.35 mg/ml in RNase free water with 25 mM sodium acetate pH 5.0 as the aqueous phase. Corresponding amount of lipid phase, which were one third in volume as the aqueous phase, were calculated with N:P ratio of 6:1 and prepared by adding the lipid solutions SM-102 (MedChemExpress, HY-134541), DSPC (Avanti, 850365), cholesterol (Sigma-Aldrich, C8667), and PEG2000 PE (Avanti, 880150) at the molar ratio of 50:10:38.5:1.5 into ethanol. Aqueous phase and lipid phase were then transferred into individual syringes and loaded to the pre-washed NanoAssemblr Benchtop Acetone Cartridge (Precision, NIT0058). LNP were formulated by mixing of the aqueous phase and lipid phase at a flow ratio of 3:1 and a flow speed of 6 ml/min. After formulation, LNP were buffer exchanged to PBS by dialysis and concentrated via ultrafiltration. mRNA encapsulation efficiencies and concentrations were determined with the Quant-iT RiboGreen RNA Assay Kit (Thermo Fisher Scientific, R11490). Diameters of LNP were measured by dynamic light scattering (DLS) using a Dynapro Naostar (Wyatt Technologies) and, finally, LNP were sterilized by filtration and stored at −80° C. in PBS with 10% sucrose.

### Immunizations, Blood Collection, and B Cell Isolation for Recipient Mice

Immunizations were initiated at 24-48 h after adoptive transfer of B cells. For protein immunization, indicated dose of conjugated or free SOSIP proteins (25 μg mi3-conjugated SOSIP per mouse in most experiments) were mixed with 20 μg MPLA (Invivogen, vac-mpls) and 10 μg Quil-A (Invivogen, vac-quil) in PBS for a total volume of 250 μl for each mouse. This antigen mixture was injected subcutaneously (s.c.) at four sites (two 50 μl injections underneath two inguinal skin, one 50 μl injection underneath abdomen skin, and one 100 μl injection underneath upper back skin). For mRNA LNP immunizations, the indicated dose of mRNA LNP (5 μg per mouse for most experiments) were diluted in 40 μl volume for each mouse, 20 μl injected into gastrocnemius muscle of each leg. Boost immunizations were administered 2 or 3 weeks, as indicated, for each experiment. Blood samples were collected one week after each immunization via submandibular bleeding. Four days after the final boost, mice were sacrificed and B cell were isolated from spleen and lymph nodes with mouse Pan B Cell Isolation Kit II (Miltenyi Biotec, 130-104-443) and LS columns (Miltenyi Biotec, 130-042-401).

### Pseudovirus Production and Neutralization Assays

HIV envelope plasmids were transformed and amplified in NEB-stable competent cells (NEB, C3040H). Pseudoviruses were produced by co-transfection of plasmids encoding various HIV-1 Envs together with NL4-3 ΔEnv or Q23-ΔEnv (1:3 ratio) in HEK293T cells using PEIpro (Polyplus, 101000033). Plasmids were acquired through the NIH HIV Reagent Program. Supernatant was harvested 48h post transfection, clarified by centrifugation and filtration with a 0.45 μm filter, and aliquoted for storage at −80° C. TZM-bl neutralization assays were performed as previously described. Briefly, titrated mouse sera or antibodies in 96-well plates were incubated with pseudotyped viruses at 37° C. for 1 hour. TZM-bl cells were then added to the wells at 10.000 cells/well. Cells were then incubated for 48 hours at 37° C. At 48 h post infection, cells were lysed in wells and subjected to firefly luciferase assays. Luciferase expression was determined using the Britelite Plus (PerkinElmer, 6066761) substrate and measured with a Victor Nivo plate reader (PerkinElmer).

### ELISA

To monitor the SOSIP-binding activity of antibodies in mouse sera by indirect ELISA, 96-well plates (Corning, 3690) were coated overnight at 4° C. with purified 16055-ConM-v8.1ds SOSIP trimers at a concentration of 5 μg/mL in PBS. Wells were washed three times with 0.05% Tween 20 in PBS, and blocked for 1 hour at room temperature with 100 μL of 3% globulin free BSA (Sigma-Aldrich, A7030). After blocking, wells were loaded with 50 μl of serially diluted mouse sera in triplicates for one hour at RT followed by five washes. Wells were then incubated with 50 μL of Fab-specific, peroxidase-labeled goat anti-Mouse IgG (Sigma-Aldrich, A9917) with the dilution for 1:2000 in 1% BSA PBS solution for 30 minutes at RT. Following incubation and five washes, 50 μL of Ultra TMB-ELISA substrate (Thermo Scientific, 34028) was added to each well and incubated at RT for 3 minutes and the reaction was terminated with 50 μL of TMB Stop solution (SouthernBiotech, 0412-01). Absorbance at 450 nm (OD450) was measured with a Victor Nivo plate reader.

For competition ELISA experiments, 96-well plates (Corning, 3690) were coated overnight at 4° C. with purified 16055-ConM-v8.1ds SOSIP trimer at a concentration of 5 μg/mL in PBS. Wells were then washed three times with 0.05% Tween 20 in PBS and blocked for 1 hour at room temperature with 100 μL of 3% globulin free BSA (Sigma-Aldrich, A7030) in PBS. Mouse sera was diluted 30-fold with 1% BSA in PBS and 50 μL of diluted sera was added in triplicate to the BSA-blocked wells. Following one-hour incubation with the mouse sera, 50 μL of 1 μg/mL of VRC26.25 or germline-VRC26 was added and mixed for the competitional binding to SOSIP trimers. Incubation of 30 minutes at RT was followed by five washes with 0.05% Tween 20 in PBS. Wells were then incubated with 50 μL of Fc specific, peroxidase labeled goat anti-human IgG (Sigma-Aldrich, A0170) with the dilution of 1:2000 in 1% BSA solution for 30 minutes at RT, and analyzed as described for indirect ELISA studies.

### Staining, Sorting, Sequencing, and Analysis of the Mouse B Cell IgH Repertoire

Engineered mouse B cells were analyzed by double-staining with FITC labeled anti-mouse IgM antibody (Miltenyi Biotec. 130-095-906) and a SOSIP protein labeled with APC using a fluorochrome using the Lightning-Link® Fluorescein Antibody Labeling Kit (Novus, 705-0030). For NGS analysis of the gene-editing events, mouse B cells were sorted with SOSIP proteins. For time course studies, mice were sacrificed at the indicated time points to harvest B cells from spleen and lymph nodes. The following reagent and antibody panel was used for flow cytometry analysis: DAPI (Biolegend, 422801), anti-CD19-PerCP/Cyanine5.5 (BioLegend, 152405), anti-CD45.1-FITC (BioLegend, 110706), anti-CD38-APC/Cyanine7 (BioLegend, 102727), anti-GL7-PE (BioLegend, 144607), and APC labeled SOSIP trimers. To analyze the repertoires of edited B cells engineered BCR, antigen-positive mouse B cells were sorted for NGS analysis. Specifically, mouse B cells were first isolated from spleen and lymph nodes using the Pan B-cell Isolation Kit II (Miltenyi Biotec, 130-104-443) following the manufacturer's protocol. Then isolated B cells were labeled with biotinylated anti-CD45.2 antibody (BioLegend. 109803), and then depleted with the anti-biotin Microbeads Ultrapure (Miltenyi Biotec, 130-105-637). Enriched B cells were finally sorted by DAPI, anti-CD45.1 antibody, and SOSIP proteins.

Sorted B cells were lysed for RNA extraction by the RNeasy Micro Kit (Qiagen, 74004). Primers used for reverse transcription and library amplification are provided in Table S2. The IgH repertoire library was prepared as previously described.¹⁵ Briefly, first-strand cDNA synthesis was performed on 11 μl of total RNA using 10 pmol of each primer in a 20 μl total reaction with SuperScript III Reverse Transcriptase (Thermo Fisher, 18080093) according to the manufacturer's protocol. DNA synthesis reaction was carried out in 100 μl using 10 pmol of each primer with HotStarTaq Plus DNA Polymerase (Qiagen, 203603). Purified dsDNA products were amplified with 10 pmol of each primer in a 100 μl total reaction volume, again with HotStarTaq Plus polymerase. Final indexing was prepared using the NEBNext Multiplex Oligos for Illumina (NEB. E7710S). All PCR products were purified by ExoSAP-IT reagent (Thermo Fisher, 78205.10.ML) and SPRI beads (Beckman Coulter Genomics, SPRIselect). Bead-purified libraries were sequenced using an Illumina MiSeq 2×250 bp paired end reads. Sequencing reads were processed and analyzed as follows: Paired-end reads were quality filtered and trimmed by Trimmomatic, then merged with PANDAseq using the default algorithm. Merged reads were collapsed by UMI through Migec using the “checkout” algorithm. Processed reads were mapped and annotated by Abstar or Mixer.

### Analysis Software

Analyzed data and sequences were plotted and graphed using python and GraphPad Prism 9. Flow cytometry data were processed using FlowJo 10. GraphPad Prism 9 was used for data analysis.

### Quantification and Statistical Analysis

Statistical information including n, mean, median and statistical significance values are indicated in the text or the figure legends. GraphPad Prism 9.01 was used to calculate significant difference by one- and two-way ANOVA, and by paired t test. Data were considered statistically significant at *p<0.05, **p<0.01, ***p<0.001, and ****p<0.0001.

For all pseudovirus neutralization assays, the IC₅₀ (the concentration of mAb needed to obtain 50% neutralization against a given pseudovirus) was calculated from the non-linear regression of the neutralization curve.

### REFERENCES

1. Hoogenboom, H. R., Selecting and screening recombinant antibody libraries. Nature biotechnology. 23, 1105-1116 (2005).
2. Liu, W., et al, Antigen affinity discrimination is an intrinsic function of the B cell receptor. Journal of Experimental Medicine. 207, 1095-1111 (2010).
3. Yang Shih, T. A., et al. Role of BCR affinity in T cell-dependent antibody responses in vivo. Nature immunology. 3, 570-575 (2002).
4. Wardemann. H., et al, Predominant autoantibody production by early human B cell precursors. Science. 301, 1374-1377 (2003).
5. Jardine, J. G., et al, Priming a broadly neutralizing antibody response to HIV-1 using a germline-targeting immunogen. Science. 349, 156-161 (2015).
6. Dosenovic, P., et al, Immunization for HIV-1 broadly neutralizing antibodies in human Ig knockin mice. Cell. 161, 1505-1515 (2015).
7. Escolano, A., et al, Sequential immunization elicits broadly neutralizing anti-HIV-1 antibodies in Ig knockin mice. Cell. 166, 1445-1458 (2016).
8. Hartweger, H., et al, HIV-specific humoral immune responses by CRISPR/Cas9-edited B cells. Journal of Experimental Medicine. 216, 1301-1310 (2019).
9. Moffett, H. F., et al, B cells engineered to express pathogen-specific antibodies protect against infection. Science immunology. 4, eaax0644 (2019).
10. Nahmad, A. D., et al, Engineered B cells expressing an anti-HIV antibody enable memory retention, isotype switching and clonal expansion. Nature communications. 11, 1-10 (2020).
11. Huang, D., et al, Vaccine elicitation of HIV broadly neutralizing antibodies from engineered B cells. Nature communications. 11, 1-10 (2020a).
12. Nahmad, A. D., et al, https://www.biorxiv.org/content/10.1101/2021.04.08.438900v1 (2021).
13. Huang, D., et al. B cells expressing authentic naive human VRC01-class BCRs can be recruited to germinal centers and affinity mature in multiple independent mouse models. Proceedings of the National Academy of Sciences. 117, 22920-22931 (2020b).
14. Jacobsen, J. T., et al. One-step generation of monoclonal B cell receptor mice capable of isotype switching and somatic hypermutation. Journal of Experimental Medicine. 215, 2686-2695 (2018).
15. Voss, J. E., et al, Reprogramming the antigen specificity of B cells using genome-editing technologies. Elife. 8, e42995 (2019).
16. Sliepen, K., et al, Structure and immunogenicity of a stabilized HIV-1 envelope trimer based on a group-M consensus sequence. Nature communications. 10, 1-16 (2019).
17. Guenaga, J., et al, Structure-guided redesign increases the propensity of HIV Env to generate highly stable soluble trimers. Journal of virology. 90, 2806-2817 (2015).
18. Mouquet, H., et al, Complex-type N-glycan recognition by potent broadly neutralizing HIV antibodies. Proceedings of the National Academy of Sciences. 109, E3268-E3277 (2012).
19. Collins, A. M., et al, The mouse antibody heavy chain repertoire is germline-focused and highly variable between inbred strains. Philosophical Transactions of the Royal Society B: Biological Sciences. 370, 20140236 (2015).
20. Yin, Y., et al, In vitro affinity maturation of broader and more-potent variants of the HIV-1-neutralizing antibody CAP256-VRC26, 25. Proceedings of the National Academy of Sciences. 118, (2021).
21. Hsia, Y., et al, Design of a hyperstable 60-subunit protein icosahedron. Nature. 535, 136-139 (2016).
22. He, L., et al, HIV-1 vaccine design through minimizing envelope metastability. Science advances. 4, eaau6769 (2018).
23. Prigent, J., et al, Conformational plasticity in broadly neutralizing HIV-1 antibodies triggers polyreactivity. Cell reports. 23, 2568-2581 (2018).
24. Diskin, R., et al, Increasing the potency and breadth of an HIV antibody by using structure-based rational design. Science. 334, 1289-1293 (2011).
25. Walker. L. M., et al. Broad neutralization coverage of HIV by multiple highly potent antibodies. Nature. 477, 466-470 (2011).
26. Smith, G. P., Petrenko, V. A, Phage display. Chemical reviews. 97, 391-410 (1997).
27. Boder, E. T., Wittrup. K. D., Yeast surface display for screening combinatorial polypeptide libraries. Nature biotechnology. 15, 553-557 (1997).
28. Beerli, R. R., et al, Isolation of human monoclonal antibodies by mammalian cell display. Proceedings of the National Academy of Sciences. 105, 14336-14341 (2008).
29. Abbott, R. K., et al, Precursor frequency and affinity determine B cell competitive fitness in germinal centers, tested with germline-targeting HIV vaccine immunogens. Immunity. 48, 133-146 (2018).
30. Rogers, G. L., Cannon, P. M., Genome edited B cells: a new frontier in immune cell therapies. Molecular Therapy. 29, 3192-3204 (2021).
31. Julg, B., et al, Broadly neutralizing antibodies targeting the HIV-1 envelope V2 apex confer protection against a clade C SHIV challenge. Science translational medicine. 9, eaal1321 (2017).
32. Garber. D. A., et al, Broadly neutralizing antibody-mediated protection of macaques against repeated intravenous exposures to simian-human immunodeficiency virus. AIDS. 35, 1567-1574 (2021).
33. Gardner, M. R., et al. Anti-drug antibody responses impair prophylaxis mediated by AAV-delivered HIV-1 broadly neutralizing antibodies. Molecular Therapy. 27, 650-660 (2019).
34. Martinez-Navio, J. M., et al, Adeno-associated virus delivery of anti-HIV monoclonal antibodies can drive long-term virologic suppression. Immunity. 50, 567-575 (2019).
35. Tung, J. W., et al, Phenotypically distinct B cell development pathways map to the three B cell lineages in the mouse. Proceedings of the National Academy of Sciences. 103, 6293-6298 (2006).
36. Mei, H. E., et al, A unique population of IgG-expressing plasma cells lacking CD19 is enriched in human bone marrow. Blood, The Journal of the American Society of Hematology. 125, 1739-1748 (2015).
37. Chen, S., et al, CRISPR-READI: efficient generation of knockin mice by CRISPR RNP electroporation and AAV donor infection. Cell reports. 27, 3780-3789 (2019).
38. Martin, R. M., et al, Highly efficient and marker-free genome editing of human pluripotent stem cells by CRISPR-Cas9 RNP and AAV6 donor-mediated homologous recombination. Cell stem cell. 24, 821-828 (2019).
39. Rogers, G. L., et al, Optimization of AAV6 transduction enhances site-specific genome editing of primary human lymphocytes. Molecular Therapy-Methods & Clinical Development. 23, 198-209 (2021).
40. Montefiori, D. C., Evaluating neutralizing antibodies against HIV, SIV, and SHIV in luciferase reporter gene assays. Current protocols in immunology. 64, 12-11 (2004).
41. Greiner. V., et al, CRISPR-mediated editing of the B cell receptor in primary human B cells. Iscience. 12, 369-378 (2019).
42. Ou, T., et al, Reprogramming of the heavy-chain CDR3 regions of a human antibody repertoire. Molecular Therapy. 30, 184-197 (2022).
43. Walker, L. M., Phogat, S. K., Chan-Hui, P. Y., Wagner, D., Phung, P., Goss, J. L., Wrin, T., Simek, M. D., Fling, S., Mitcham, J. L., et al. (2009). Broad and potent neutralizing antibodies from an African donor reveal a new HIV-1 vaccine target. Science 326, 285-289, 10.1126/science.1178746.
44. Wu. X., Yang, Z. Y., Li, Y., Hogerkorp, C. M., Schief, W. R., Seaman. M. S., Zhou. T., Schmidt, S. D., Wu, L., Xu, L., et al. (2010). Rational design of envelope identifies broadly neutralizing human monoclonal antibodies to HIV-1. Science 329, 856-861, 10.1126/science.1187659.
45. Liao, H. X., Lynch, R., Zhou, T., Gao, F., Alam, S. M., Boyd, S. D., Fire, A. Z., Roskin, K. M., Schramm, C. A., Zhang, Z., et al. (2013). Co-evolution of a broadly neutralizing HIV-1 antibody and founder virus. Nature 496, 469-476, 10.1038/nature12053.
46. van Gils, M. J., and Sanders, R. W. (2013). Broadly neutralizing antibodies against HIV-1: templates for a vaccine. Virology 435, 46-56, 10.1016/j.virol.2012.10.004.
47. Halper-Stromberg, A., Lu. C. L., Klein, F., Horwitz, J. A., Bournazos, S., Nogueira, L., Eisenreich, T. R., Liu, C., Gazumyan, A., Schaefer, U., et al. (2014). Broadly neutralizing antibodies and viral inducers decrease rebound from HIV-1 latent reservoirs in humanized mice. Cell 158, 989-999, 10.1016/j.cell.2014.07.043.
48. West, A. P., Jr., Scharf, L., Scheid, J. F., Klein, F., Bjorkman, P. J., and Nussenzweig, M. C. (2014). Structural insights on the role of antibodies in HIV-1 vaccine and therapy. Cell 156, 633-648, 10.1016/j.cell.2014.01.052.
49. Klasse, P. J., LaBranche, C. C., Ketas, T. J., Ozorowski, G., Cupo, A., Pugach, P., Ringe, R. P., Golabek, M., van Gils, M. J., Guttman, M., et al. (2016). Sequential and Simultaneous Immunization of Rabbits with HIV-1 Envelope Glycoprotein SOSIP.664 Trimers from Clades A, B and C. PLoS Pathog 12, e1005864, 10.1371/journal.ppat.1005864.
50. Steichen, J. M., Kulp, D. W., Tokatlian, T., Escolano, A., Dosenovic, P., Stanfield, R. L., McCoy, L. E., Ozorowski, G., Hu, X., Kalyuzhniy, O., et al. (2016). HIV Vaccine Design to Target Germline Precursors of Glycan-Dependent Broadly Neutralizing Antibodies. Immunity 45, 483-496, 10.1016/j.immuni.2016.08.016.
51. Tian, M., Cheng, C., Chen, X., Duan, H., Cheng, H. L., Dao, M., Sheng, Z., Kimble, M., Wang, L., Lin, S., et al. (2016). Induction of HIV Neutralizing Antibody Lineages in Mice with Diverse Precursor Repertoires. Cell 166, 1471-1484 e1418, 10.1016/j.cell.2016.07.029.
52. Escolano, A., Steichen, J. M., Dosenovic, P., Kulp, D. W., Golijanin, J., Sok, D., Freund, N. T., Gitlin, A. D., Oliveira, T., Araki, T., et al. (2016). Sequential Immunization Elicits Broadly Neutralizing Anti-HIV-1 Antibodies in Ig Knockin Mice. Cell 166, 1445-1458 e1412, 10.1016/j.cell.2016.07.030.
53. Mclellan, J. S., Pancera, M., Carrico, C., Gorman, J., Julien, J. P., Khayat, R., Louder, R., Pejchal, R., Sastry, M., Dai, K., et al. (2011). Structure of HIV-1 gp120 V1/V2 domain with broadly neutralizing antibody PG9. Nature 480, 336-343, 10.1038/nature10696.
54. Andrabi, R., Voss, J. E., Liang, C. H., Briney, B., McCoy, L. E., Wu, C. Y., Wong, C. H., Poignard. P., and Burton, D. R. (2015). Identification of Common Features in Prototype Broadly Neutralizing Antibodies to HIV Envelope V2 Apex to Facilitate Vaccine Design. Immunity 43, 959-973, 10.1016/j.immuni.2015.10.014.
55. Lee. J. H., Andrabi, R., Su, C. Y., Yasmeen, A., Julien, J. P., Kong, L., Wu, N. C., McBride, R., Sok, D., Pauthner, M., et al. (2017). A Broadly Neutralizing Antibody Targets the Dynamic HIV Envelope Trimer Apex via a Long, Rigidified, and Anionic beta-Hairpin Structure. Immunity 46, 690-702, 10.1016/j.immuni.2017.03.017.
56. Voss. J., Andrabi, R., McCoy, L., de Val, N., Fuller, R., Messmer, T., Su, C., Sok, D., Khan, S., and Garces, F. (2017). Elicitation of neutralizing antibodies targeting the V2 apex of the HIV envelope trimer in a wild-type animal model. Cell Rep
57. Briney, B., Inderbitzin, A., Joyce, C., and Burton, D. R. (2019). Commonality despite exceptional diversity in the baseline human antibody repertoire. Nature 566, 393-397, 10.1038/s41586-019-0879-y.
58. Roark, R. S., Li, H., Williams, W. B., Chug. H., Mason, R. D., Gorman, J., Wang, S., Lee, F. H., Rando, J., Bonsignori. M., et al. (2021). Recapitulation of HIV-1 Env-antibody coevolution in macaques leading to neutralization breadth. Science 371, eabd2638, 10.1126/science.abd2638.
59. Walker, L. M., Simek, M. D., Priddy, F., Gach, J. S., Wagner, D., Zwick, M. B., Phogat, S. K., Poignard, P., and Burton, D. R. (2010). A limited number of antibody specificities mediate broad and potent serum neutralization in selected HIV-1 infected individuals. PLoS Pathog 6, e1001028, 10.1371/journal.ppat. 1001028.
60. Georgiev, I. S., Doria-Rose, N. A., Zhou, T., Kwon, Y. D., Staupe, R. P., Moquin, S., Chuang, G. Y., Louder, M. K., Schmidt, S. D., Altae-Tran, H. R., et al. (2013). Delineating antibody recognition in polyclonal sera from patterns of HIV-1 isolate neutralization. Science 340, 751-756, 10.1126/science. 1233989.
61. Doria-Rose, N. A., Schramm, C. A., Gorman, J., Moore, P. L., Bhiman, J. N., DeKosky, B. J., Ernandes, M. J., Georgiev, I. S., Kim, H. J., Pancera, M., et al. (2014). Developmental pathway for potent V1V2-directed HIV-neutralizing antibodies. Nature 509, 55-62, 10.1038/nature13036.
62. Doria-Rose, N. A., Bhiman, J. N., Roark, R. S., Schramm, C. A., Gorman, J., Chuang, G.-Y., Pancera, M., Cale, E. M., Ernandes, M. J., and Louder, M. K. (2016). New member of the V1V2-directed CAP256-VRC26 lineage that shows increased breadth and exceptional potency. Journal of virology 90, 76-91.
63. Pancera, M., Shahzad-Ul-Hussan, S., Doria-Rose, N. A., McLellan, J. S., Bailer, R. T., Dai, K., Loesgen, S., Louder, M. K., Staupe, R. P., Yang, Y., et al. (2013). Structural basis for diverse N-glycan recognition by HIV-1-neutralizing V1-V2-directed antibody PG16. Nat Struct Mol Biol 20, 804-813, 10.1038/nsmb.2600.
64. Sok, D., van Gils, M. J., Pauthner, M., Julien, J. P., Saye-Francisco, K. L., Hsueh, J., Briney, B., Lee, J. H., Le, K. M., Lee, P. S., et al. (2014). Recombinant HIV envelope trimer selects for quaternary-dependent antibodies targeting the trimer apex. Proc Natl Acad Sci USA 111, 17624-17629, 10.1073/pnas. 1415789111.
65. Sanders, R. W., van Gils, M. J., Derking, R., Sok, D., Ketas, T. J., Burger, J. A., Ozorowski, G., Cupo. A., Simonich, C., Goo, L., et al. (2015). HIV-1 VACCINES. HIV-1 neutralizing antibodies induced by native-like envelope trimers. Science 349, aac4223, 10.1126/science.aac4223.
66. Ringe, R. P., Yasmeen, A., Ozorowski, G., Go, E. P., Pritchard, L. K., Guttman, M., Ketas, T. A., Cottrell, C. A., Wilson, I. A., Sanders, R. W., et al. (2015). Influences on the Design and Purification of Soluble, Recombinant Native-Like HIV-1 Envelope Glycoprotein Trimers. J Virol 89, 12189-12210, 10.1128/JVI.01768-15.
67. Cupo. A., Cruz Portillo, V. M., Gelfand, P., Yasmeen, A., Klasse, P. J., and Moore, J. P. (2019). Optimizing the production and affinity purification of HIV-1 envelope glycoprotein SOSIP trimers from transiently transfected CHO cells. PLoS One 14, e0215106, 10.1371/journal.pone.0215106.
68. Richardson, S. I., Chung, A. W., Natarajan, H., Mabvakure, B., Mkhize, N. N., Garrett, N., Abdool Karim, S., Moore, P. L., Ackerman, M. E., Alter, G., and Morris, L. (2018). HIV-specific Fc effector function early in infection predicts the development of broadly neutralizing antibodies. PLoS Pathog 14, e1006987, 10.1371/journal.ppat.1006987.
69. Sanders, R. W., Derking, R., Cupo, A., Julien, J. P., Yasmeen, A., de Val, N., Kim, H. J., Blattner, C., de la Pena, A. T., Korzun. J., et al. (2013). A next-generation cleaved, soluble HIV-1 Env trimer, BG505 SOSIP.664 gp140, expresses multiple epitopes for broadly neutralizing but not non-neutralizing antibodies. PLoS Pathog 9, e1003618, 10.1371/journal.ppat.1003618.
70. Guenaga, J., Dubrovskaya. V., de Val, N., Sharma, S. K., Carrette, B., Ward, A. B., and Wyatt, R. T. (2015). Structure-Guided Redesign Increases the Propensity of HIV Env To Generate Highly Stable Soluble Trimers. J Virol 90, 2806-2817, 10.1128/JVI.02652-15.
71. Medina-Ramirez, M., Garces, F., Escolano, A., Skog, P., de Taeye, S. W., Del Moral-Sanchez, I., McGuire, A. T., Yasmeen, A., Behrens, A. J., Ozorowski, G., et al. (2017). Design and crystal structure of a native-like HIV-1 envelope trimer that engages multiple broadly neutralizing antibody precursors in vivo. J Exp Med 214, 2573-2590, 10.1084/jem.20161160.
72. Guenaga, J., Garces, F., de Val, N., Stanfield, R. L., Dubrovskaya, V., Higgins, B., Carrette, B., Ward, A. B., Wilson, I. A., and Wyatt, R. T. (2017). Glycine Substitution at Helix-to-Coil Transitions Facilitates the Structural Determination of a Stabilized Subtype C HIV Envelope Glycoprotein. Immunity 46, 792-803 e793, 10.1016/j.immuni.2017.04.014.
73. Kwon, Y. D., Pancera, M., Acharya, P., Georgiev, I. S., Crooks, E. T., Gorman, J., Joyce, M. G., Guttman, M., Ma, X., Narpala. S., et al. (2015). Crystal structure, conformational fixation and entry-related interactions of mature ligand-free HIV-1 Env. Nat Struct Mol Biol 22, 522-531, 10.1038/nsmb.3051.
74. Rawi, R., Rutten, L., Lai, Y. T., Olia, A. S., Blokland, S., Juraszek, J., Shen, C. H., Tsybovsky, Y., Verardi, R., Yang, Y., et al. (2020). Automated Design by Structure-Based Stabilization and Consensus Repair to Achieve Prefusion-Closed Envelope Trimers in a Wide Variety of HIV Strains. Cell Rep 33, 108432. 10.1016/j.celrep.2020.108432.
75. Martinez-Murillo, P., Tran, K., Guenaga, J., Lindgren, G., Adori, M., Feng, Y., Phad, G. E., Bernat, N. V., Bale, S., and Ingale, J. (2017). Particulate array of well-ordered HIV clade C Env trimers elicits neutralizing antibodies that display a unique V2 cap approach. Immunity 46, 804-817. e807.
76. Sharma, S. K., de Val, N., Bale, S., Guenaga, J., Tran, K., Feng, Y., Dubrovskaya, V., Ward, A. B., and Wyatt, R. T. (2015). Cleavage-independent HIV-1 Env trimers engineered as soluble native spike mimetics for vaccine design. Cell reports 11, 539-550.
77. Georgiev, I. S., Joyce, M. G., Yang, Y., Sastry, M., Zhang, B., Baxa, U., Chen, R. E., Druz, A., Lees, C. R., Narpala, S., et al. (2015). Single-Chain Soluble BG505.SOSIP gp140 Trimers as Structural and Antigenic Mimics of Mature Closed HIV-1 Env. J Virol 89, 5318-5329. 10.1128/JVI.03451-14.
78. He, L., Kumar. S., Allen, J. D., Huang, D., Lin, X., Mann, C. J., Saye-Francisco, K. L., Copps, J., Sarkar, A., Blizard, G. S., et al. (2018). HIV-1 vaccine design through minimizing envelope metastability. Sci Adv 4, eaau6769. 10.1126/sciadv.aau6769.
79. Aldon, Y., Mckay, P. F., Allen, J., Ozorowski, G., Felfodine Levai, R., Tolazzi, M., Rogers, P., He. L., de Val, N., Fabian, K., et al. (2018). Rational Design of DNA-Expressed Stabilized Native-Like HIV-1 Envelope Trimers. Cell Rep 24, 3324-3338 e3325. 10.1016/j.celrep.2018.08.051.
80. McCoy, L. E., van Gils, M. J., Ozorowski, G., Messmer, T., Briney, B., Voss, J. E., Kulp, D. W., Macauley. M. S., Sok, D., Pauthner, M., et al. (2016). Holes in the Glycan Shield of the Native HIV Envelope Are a Target of Trimer-Elicited Neutralizing Antibodies. Cell Rep 16, 2327-2338. 10.1016/j.celrep.2016.07.074.
81. Brouwer, P. J. M., Antanasijevic, A., Berndsen, Z., Yasmeen, A., Fiala, B., Bijl. T. P. L., Bontjer. I., Bale, J. B., Sheffler, W., Allen, J. D., et al. (2019). Enhancing and shaping the immunogenicity of native-like HIV-1 envelope trimers with a two-component protein nanoparticle. Nat Commun 10, 4272. 10.1038/s41467-019-12080-1.
82. Sliepen, K., Han, B. W., Bontjer, I., Mooij, P., Garces, F., Behrens, A. J., Rantalainen, K., Kumar, S., Sarkar, A., Brouwer, P. J. M., et al. (2019). Structure and immunogenicity of a stabilized HIV-1 envelope trimer based on a group-M consensus sequence. Nat Commun 10, 2355. 10.1038/s41467-019-10262-5.
83. Chuang, G. Y., Geng, H., Pancera, M., Xu, K., Cheng, C., Acharya, P., Chambers, M., Druz, A., Tsybovsky. Y., Wanninger, T. G., et al. (2017). Structure-Based Design of a Soluble Prefusion-Closed HIV-1 Env Trimer with Reduced CD4 Affinity and Improved Immunogenicity. J Virol 91, e02268-02216. 10.1128/JVI.02268-16.
84. Pardi, N., Hogan, M. J., and Weissman, D. (2020). Recent advances in mRNA vaccine technology. Curr Opin Immunol 65, 14-20. 10.1016/j.coi.2020.01.008.
85. Saunders, K. O., Pardi. N., Parks, R., Santra. S., Mu, Z., Sutherland, L., Scearce, R., Barr, M., Eaton, A., and Hernandez, G. (2021). Lipid nanoparticle encapsulated nucleoside-modified mRNA vaccines elicit polyfunctional HIV-1 antibodies comparable to proteins in nonhuman primates. npj Vaccines 6, 1-14.
86. Zhang, P., Narayanan, E., Liu, Q., Tsybovsky, Y., Boswell, K., Ding, S., Hu, Z., Follmann, D., Lin, Y., Miao, H., et al. (2021). A multiclade env-gag VLP mRNA vaccine elicits tier-2 HIV-1-neutralizing antibodies and reduces the risk of heterologous SHIV infection in macaques. Nat Med 27, 2234-2245. 10.1038/s41591-021-01574-5.
87. Doms, R. W . . . and Moore, J. P. (2000). HIV-1 membrane fusion: targets of opportunity. J Cell Biol 151, F9-14. 10.1083/jcb.151.2.f9.
88. de Taeye. S. W., Ozorowski, G., Torrents de la Pena, A., Guttman. M., Julien, J. P., van den Kerkhof, T. L., Burger, J. A., Pritchard, L. K., Pugach, P., Yasmeen, A., et al. (2015). Immunogenicity of Stabilized HIV-1 Envelope Trimers with Reduced Exposure of Non-neutralizing Epitopes. Cell 163. 1702-1715. 10.1016/j.cell.2015.11.056.
89. Turner. H. L., Andrabi, R., Cottrell. C. A., Richey. S. T., Song, G., Callaghan, S., Anzanello, F., Moyer, T. J., Abraham, W., and Melo. M. (2021). Disassembly of HIV envelope glycoprotein trimer immunogens is driven by antibodies elicited via immunization. Science Advances 7, eabh2791.
90. van Schooten, J., van Haaren, M. M., Li, H., McCoy, L. E., Havenar-Daughton, C., Cottrell, C. A., Burger, J. A., van der Woude. P., Helgers, L. C., and Tomris. I. (2021). Antibody responses induced by SHIV infection are more focused than those induced by soluble native HIV-1 envelope trimers in non-human primates. PLoS pathogens 17, e1009736.
91. Lefranc, M. P., Giudicelli. V., Duroux, P., Jabado-Michaloud, J., Folch. G., Aouinti, S., Carillon, E., Duvergey, H., Houles. A., Paysan-Lafosse, T., et al. (2015). IMGT®, the international ImMunoGeneTics Information System® 25 years on. Nucleic Acids Res 43, D413-422. 10.1093/nar/gku1056.
92. Lefranc, M. P. (2014). Immunoglobulin and T Cell Receptor Genes: IMGT® and the Birth and Rise of Immunoinformatics. Front Immunol 5, 22. 10.3389/fimmu.2014.00022.
93. Crooks, E. T., Almanza, F., D'Addabbo, A., Duggan, E., Zhang, J., Wagh, K., Mou, H., Allen, J. D., Thomas, A., Osawa, K., et al. (2021). Engineering well-expressed. V2-immunofocusing HIV-1 envelope glycoprotein membrane trimers for use in heterologous prime-boost vaccine regimens. PLoS Pathog 17, e1009807. 10.1371/journal.ppat.1009807.
94. Melzi, E., Willis, J. R., Ma, K. M., Lin, Y. C., Kratochvil, S., Berndsen, Z. T., Landais, E. A., Kalyuzhniy, O., Nair, U., Warner, J., et al. (2022). Membrane-bound mRNA immunogens lower the threshold to activate HIV Env V2 apex-directed broadly neutralizing B cell precursors in humanized mice. Immunity 55, 2168-2186 e2166. 10.1016/j.immuni.2022.09.003.
95. Hartweger, H., McGuire. A. T., Horning. M., Taylor, J. J., Dosenovic. P., Yost, D., Gazumyan, A., Seaman, M. S., Stamatatos, L., Jankovic, M., and Nussenzweig. M. C. (2019). HIV-specific humoral immune responses by CRISPR/Cas9-edited B cells. J Exp Med 216, 1301-1310. 10.1084/jem.20190287.
96. Voss, J. E., Gonzalez-Martin, A., Andrabi, R., Fuller, R. P., Murrell, B., McCoy, L. E., Porter, K., Huang, D., Li, W., Sok, D., et al. (2019). Reprogramming the antigen specificity of B cells using genome-editing technologies. Elife 8, e42995. 10.7554/eLife.42995.
97. Huang, D., Tran. J. T., Olson, A., Vollbrecht, T., Tenuta, M., Guryleva, M. V., Fuller, R. P., Schiffner, T., Abadejos. J. R., and Couvrette. L. (2020). Vaccine elicitation of HIV broadly neutralizing antibodies from engineered B cells. Nature communications 11, 1-10.
98. Moffett, H. F., Harms, C. K., Fitzpatrick, K. S., Tooley, M. R., Boonyaratanakornkit, J., and Taylor, J. J. (2019). B cells engineered to express pathogen-specific antibodies protect against infection. Sci Immunol 4. 10.1126/sciimmunol.aax0644.
99. Nahmad, A. D., Raviv. Y., Horovitz-Fried. M., Sofer, I., Akriv, T., Nataf, D., Dotan. I., Carmi, Y., Burstein, D., Wine, Y., et al. (2020). Engineered B cells expressing an anti-HIV antibody enable memory retention, isotype switching and clonal expansion. Nat Commun 11. 5851. 10.1038/s41467-020-19649-1.
100. Nahmad, A. D., Lazzarotto, C. R., Zelikson, N., Kustin, T., Tenuta, M., Huang, D., Reuveni, I., Nataf, D., Raviv, Y., Horovitz-Fried, M., et al. (2022). In vivo engineered B cells secrete high titers of broadly neutralizing anti-HIV antibodies in mice. Nat Biotechnol 40. 1241-1249. 10.1038/s41587-022-01328-9.
101. Ou, T., He, W., Quinlan. B. D., Guo, Y., Tran, M. H., Karunadharma, P., Park. H., Davis-Gardner, M. E., Yin, Y., Zhang. X., et al. (2022). Reprogramming of the heavy-chain CDR3 regions of a human antibody repertoire. Mol Ther 30, 184-197. 10.1016/j.ymthe.2021.10.027.
102. Keeble, A. H., Turkki, P., Stokes, S., Khairil Anuar, I. N., Rahikainen, R., Hytönen, V. P., and Howarth, M. (2019). Approaching infinite affinity through engineering of peptide-protein interaction. Proceedings of the National Academy of Sciences 116, 26523-26533.
103. Bruun, T. U. J., Andersson, A. C., Draper, S. J., and Howarth. M. (2018). Engineering a Rugged Nanoscaffold To Enhance Plug-and-Display Vaccination. ACS Nano 12, 8855-8866. 10.1021/acsnano.8b02805.
104. Yin, Y., Quinlan, B. D., Ou. T., Guo. Y., He, W., and Farzan, M. (2021). In vitro affinity maturation of broader and more-potent variants of the HIV-1-neutralizing antibody CAP256-VRC26.25. Proc Natl Acad Sci USA 118, e2106203118. 10.1073/pnas.2106203118.
105. Brouwer, P. J. M., Antanasijevic, A., de Gast, M., Allen, J. D., Bijl, T. P. L., Yasmeen, A., Ravichandran, R., Burger. J. A., Ozorowski, G., Torres, J. L., et al. (2021). Immunofocusing and enhancing autologous Tier-2 HIV-1 neutralization by displaying Env trimers on two-component protein nanoparticles. NPJ Vaccines 6, 24, 10.1038/s41541-021-00285-9.
106. Antanasijevic, A., Ueda. G., Brouwer, P. J. M., Copps, J., Huang, D., Allen, J. D., Cottrell, C. A., Yasmeen, A., Sewall, L. M., Bontjer. I., et al. (2020). Structural and functional evaluation of de novo-designed, two-component nanoparticle carriers for HIV Env trimer immunogens. PLoS Pathog 16, e1008665. 10.1371/journal.ppat.1008665.
107. Joyce, M. G., Georgiev, I. S., Yang, Y., Druz. A., Geng, H., Chuang, G. Y., Kwon. Y. D., Pancera. M., Rawi. R., Sastry. M., et al. (2017). Soluble Prefusion Closed DS-SOSIP.664-Env Trimers of Diverse HIV-1 Strains. Cell Rep 21, 2992-3002. 10.1016/j.celrep.2017.11.016.
108. Chuang. G.-Y., Lai, Y.-T., Boyington, J. C., Cheng. C., Geng. H., Narpala, S., Rawi, R., Schmidt, S. D., Tsybovsky, Y., and Verardi, R. (2020). Development of a 3mut-apex-stabilized envelope trimer that expands HIV-1 neutralization breadth when used to boost fusion peptide-directed vaccine-elicited responses. Journal of Virology 94, e00074-00020.
109. Eggink, D., Melchers, M., Wuhrer, M., van Montfort. T., Dey, A. K., Naaijkens, B. A., David, K. B., Le Douce, V., Deelder, A. M., Kang. K . . . et al. (2010). Lack of complex N-glycans on HIV-1 envelope glycoproteins preserves protein conformation and entry function. Virology 401, 236-247. 10.1016/j.virol.2010.02.019.
110. Brouwer, P. J. M., Brinkkemper, M., Maisonnasse, P., Dereuddre-Bosquet. N., Grobben, M., Claireaux, M., de Gast, M., Marlin, R., Chesnais, V., Diry. S., et al. (2021). Two-component spike nanoparticle vaccine protects macaques from SARS-COV-2 infection. Cell 184, 1188-1200 e1119. 10.1016/j.cell.2021.01.035.
111. Gristick, H. B., Hartweger, H., Loewe, M., van Schooten, J., Ramos, V., Oliviera, T. Y., Nishimura, Y., Koranda, N. S., Wall, A., Yao, K.-H., et al. (2022). CD4-binding site immunogens elicit heterologous anti-HIV-1 neutralizing antibodies in transgenic and wildtype animals, bioRxiv, 2022.2009.2008.507086. 10.1101/2022.09.08.507086.
112. Laczko, D., Hogan, M. J., Toulmin, S. A., Hicks, P., Lederer, K., Gaudette, B. T., Castano, D., Amanat. F., Muramatsu, H., Oguin. T. H., 3rd, et al. (2020). A Single Immunization with Nucleoside-Modified mRNA Vaccines Elicits Strong Cellular and Humoral Immune Responses against SARS-COV-2 in Mice. Immunity 53, 724-732 e727. 10.1016/j.immuni.2020.07.019.
113. Willis, J. R., Berndsen, Z. T., Ma, K. M., Steichen, J. M., Schiffner, T., Landais, E., Liguori, A., Kalyuzhniy, O., Allen, J. D., Baboo, S., et al. (2022). Human immunoglobulin repertoire analysis guides design of vaccine priming immunogens targeting HIV V2-apex broadly neutralizing antibody precursors. Immunity 55, 2149-2167 e2149. 10.1016/j.immuni.2022.09.001.
114. Johnson, E. L., Doria-Rose, N. A., Gorman, J., Bhiman, J. N., Schramm, C. A., Vu, A. Q., Law, W. H., Zhang, B., Bekker. V., Abdool Karim, S. S., et al. (2018). Sequencing HIV-neutralizing antibody exons and introns reveals detailed aspects of lineage maturation. Nat Commun 9, 4136. 10.1038/s41467-018-06424-6.
115. Pejchal, R., Walker, L. M., Stanfield, R. L., Phogat, S. K., Koff, W. C., Poignard, P., Burton, D. R., and Wilson, I. A. (2010). Structure and function of broadly reactive antibody PG16 reveal an H3 subdomain that mediates potent neutralization of HIV-1. Proc Natl Acad Sci USA 107, 11483-11488. 10.1073/pnas. 1004600107.
116. Farzan, M., Mirzabekov, T., Kolchinsky, P., Wyatt, R., Cayabyab, M., Gerard, N. P., Gerard. C., Sodroski, J., and Choe. H. (1999). Tyrosine sulfation of the amino terminus of CCR5 facilitates HIV-1 entry. Cell 96, 667-676. 10.1016/s0092-8674 (00) 80577-2.
117. Choe, H., Li, W., Wright, P. L., Vasilieva, N., Venturi, M., Huang, C. C., Grundner, C., Dorfman, T., Zwick, M. B., Wang. L., et al. (2003). Tyrosine sulfation of human antibodies contributes to recognition of the CCR5 binding region of HIV-1 gp120. Cell 114, 161-170. 10.1016/s0092-8674 (03) 00508-7.
118. Choe, H., and Farzan, M. (2009). Chapter 7. Tyrosine sulfation of HIV-1 coreceptors and other chemokine receptors. Methods Enzymol 461, 147-170. 10.1016/S0076-6879 (09) 05407-X.
119. Monigatti, F., Gasteiger, E., Bairoch, A., and Jung, E. (2002). The Sulfinator: predicting tyrosine sulfation sites in protein sequences. Bioinformatics 18, 769-770. 10.1093/bioinformatics/18.5.769.
120. Jardine, J. G., Kulp. D. W., Havenar-Daughton. C., Sarkar, A., Briney, B., Sok, D., Sesterhenn, F., Ereno-Orbea, J., Kalyuzhniy, O., Deresa, I., et al. (2016). HIV-1 broadly neutralizing antibody precursor B cells revealed by germline-targeting immunogen. Science 351, 1458-1463. 10.1126/science.aad9195.
121. Sok, D., Briney, B., Jardine, J. G., Kulp, D. W., Menis. S., Pauthner, M., Wood, A., Lee, E. C., Le, K. M., Jones. M., et al. (2016). Priming HIV-1 broadly neutralizing antibody precursors in human Ig loci transgenic mice. Science 353, 1557-1560. 10.1126/science.aah3945.
122. Dosenovic, P., von Boehmer, L., Escolano, A., Jardine, J., Freund, N. T., Gitlin, A. D., McGuire, A. T., Kulp, D. W., Oliveira, T., Scharf, L., et al. (2015). Immunization for HIV-1 Broadly Neutralizing Antibodies in Human Ig Knockin Mice. Cell 161, 1505-1515. 10.1016/j.cell.2015.06.003.
123. Gorman, J., Chuang, G. Y., Lai, Y. T., Shen, C. H., Boyington, J. C., Druz, A., Geng, H., Louder, M. K., McKee, K., Rawi, R., et al. (2020). Structure of Super-Potent Antibody CAP256-VRC26.25 in Complex with HIV-1 Envelope Reveals a Combined Mode of Trimer-Apex Recognition. Cell Rep 31, 107488. 10.1016/j.celrep.2020.03.052.

### OTHER EMBODIMENTS

In some embodiments, the present disclosure provides:
1. A method, comprising:
   contacting a B cell obtained from a mammalian subject with a homology-directed repair (HDR) template comprising a sequence encoding heavy chain variable region CDR3 of a human antibody, a Cas protein, and a guide RNA.
   wherein the B cell comprises a genomic locus encoding a B cell receptor (BCR), and wherein the guide RNA comprises a sequence having complementarity to the genomic locus;
   whereby the Cas protein introduces a double-strand DNA break immediately adjacent to a target site in the genomic locus, and the target site is replaced with the first sequence through HDR, thereby generating an engineered BCR; and
   administering the B cell comprising the engineered BCR to the subject.
2. A method of evaluating the immunogenicity of an antigen, comprising:
   I. contacting a B cell with a homology-directed repair (HDR) template comprising a sequence encoding heavy chain variable region CDR3 of a human antibody that specifically binds the antigen, a Cas protein, and a guide RNA,
   wherein the B cell comprises a genomic locus encoding a B cell receptor (BCR), and wherein the guide RNA comprises a sequence having complementarity to the genomic locus;
   whereby the Cas protein introduces a double-strand DNA break immediately adjacent to a target site in the genomic locus, and the target site is replaced with the first sequence through HDR, thereby generating an engineered BCR;
   II. administering the B cell comprising the engineered BCR to a mammalian subject;
   III. administering the antigen to the mammalian subject, and
   IV. measuring proliferation, class-switching, and/or affinity maturation of the engineered B cell or BCR.
3. The method of paragraph 1 or 2, wherein the replacement of the target site with the sequence does not result in integration of any exogenous genetic regulatory elements into the locus encoding the BCR.
4. The method of any of the preceding paragraphs, wherein the mammalian subject is a rodent.
5. The method of any of the preceding paragraphs, wherein the mammalian subject is a wild-type mouse.
6. The method of any of the preceding paragraphs, wherein the mammalian subject is not a transgenic mouse.
7. The method of any of the preceding paragraphs, wherein the Cas protein is Cas9, Cas 12a or Cas 13.
8. The method of any one of paragraphs 1-7, whereby the method generates an affinity-matured antibody in the subject that comprises a variant of the heavy chain variable region CDR3 of the human antibody.
9. The method of any one of paragraphs 1-8, whereby the method results in somatic hypermutation and affinity maturation in the subject.
10. The method of any one of paragraphs 1-9, wherein the affinity-matured antibody has enhanced affinity to the antigenic target relative to the human antibody.
11. The method of any one of paragraphs 1-9, wherein the affinity-matured antibody has enhanced bioavailability in the subject relative to the human antibody.
12. The method of any of the preceding paragraphs, wherein the antigen is a soluble protein antigen, a transmembrane protein antigen, or a viral antigen, optionally an HIV antigen.
13. The method of paragraph 12, wherein the antigen is an HIV-1 Env trimer.
14. The method of paragraph 13, wherein the antigen comprises the V2-glycan apex epitope.
13. The method of any of the preceding paragraphs, wherein the human antibody is VRC26.25 or a variant thereof.
14. The method of any of the preceding paragraphs, wherein the HDR template (HDRT) is comprised within a double-stranded DNA (dsDNA) vector.
15. The method of any of the preceding paragraphs, wherein the HDR template (HDRT) is comprised within an adeno-associated viral (AAV) vector.
16. The method of paragraph 15, wherein the AAV vector is encapsidated in an AAV6 or AAV-DJ capsid.
17. The method of any of the preceding paragraphs, wherein the guide RNA comprises a sequence of between 15 and 200 nucleotides that is complementary to the genomic locus.

### EQUIVALENTS AND SCOPE

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents, and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive. i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of,” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently. “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

Unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures. Section 2111.03. It should be appreciated that embodiments described in this document using an open-ended transitional phrase (e.g., “comprising”) are also contemplated, in alternative embodiments, as “consisting of” and “consisting essentially of” the feature described by the open-ended transitional phrase. For example, if the disclosure describes “a composition comprising A and B,” the disclosure also contemplates the alternative embodiments “a composition consisting of A and B” and “a composition consisting essentially of A and B.”

What is claimed is:

1. A method, comprising:
contacting a B cell obtained from a mammalian subject with a homology-directed repair (HDR) template comprising a first sequence encoding heavy chain variable genes of a human antibody, a Cas protein, and a guide RNA,
wherein the B cell comprises a genomic locus encoding a B cell receptor (BCR), and wherein the guide RNA comprises a sequence having complementarity to the genomic locus;
whereby the Cas protein introduces a double-strand DNA break immediately adjacent to a target site in the genomic locus, and the target site is replaced with the first sequence through HDR, thereby generating an engineered BCR; and
administering the B cell comprising the engineered BCR to the subject.

2. A method, comprising:
contacting a B cell obtained from a mammalian subject with a homology-directed repair (HDR) template comprising a first sequence encoding light chain variable genes of a human antibody, a Cas protein, and a guide RNA,
wherein the B cell comprises a genomic locus encoding a B cell receptor (BCR), and wherein the guide RNA comprises a sequence having complementarity to the genomic locus;
whereby the Cas protein introduces a double-strand DNA break immediately adjacent to a target site in the genomic locus, and the target site is replaced with the first sequence through HDR, thereby generating an engineered BCR; and
administering the B cell comprising the engineered BCR to the subject.

3. A method, comprising:
contacting a B cell obtained from a mammalian subject with a homology-directed repair (HDR) template comprising a first sequence encoding heavy chain variable genes of a human antibody and a second sequence encoding light chain variable genes of a human antibody, a Cas protein, and a guide RNA,
wherein the B cell comprises a genomic locus encoding a B cell receptor (BCR), and wherein the guide RNA comprises a sequence having complementarity to the genomic locus;
whereby the Cas protein introduces a double-strand DNA break immediately adjacent to a target site in the genomic locus, and the target site is replaced with the first sequence through HDR, thereby generating an engineered BCR; and
administering the B cell comprising the engineered BCR to the subject.

4. The method of any of claims 1-3, wherein the replacement of the target site with the first sequence does not result in integration of any exogenous genetic regulatory elements into the locus encoding the BCR.

5. The method of any of the preceding claims, wherein the replacement of the target site with the first sequence or the second sequence does not result in integration of the first sequence or the second sequence into an intronic sequence.

6. The method of any of the preceding claims, wherein the mammalian subject is a rodent.

7. The method of any of the preceding claims, wherein the mammalian subject is a wild-type mouse.

8. The method of any of the preceding claims, wherein the mammalian subject is not a transgenic mouse.

9. The method of any of the preceding claims, wherein the Cas protein is Cas9, Cas12a or Cas 13.

10. The method of any one of claims 1-9, whereby the method generates an affinity-matured antibody in the subject that is a variant of the human antibody.

11. The method of any one of claims 1-10, whereby the method results in somatic hypermutation and affinity maturation in the subject.

12. The method of claim 11, whereby the method provides rates of somatic hypermutation of about 0.5%-4%.

13. The method of any one of claims 1-12, wherein the affinity-matured antibody has enhanced affinity to an antigenic target relative to the human antibody.

14. The method of any one of claims 1-12, wherein the affinity-matured antibody has enhanced bioavailability in the subject relative to the human antibody.

15. The method of any of the preceding claims, wherein the heavy chain variable genes of the human antibody comprises recombined germline VDJ segments and/or the light chain variable genes comprise recombined germline VJ segments.

16. The method of any of the preceding claims, wherein the heavy chain of the human antibody comprises a heavy-chain complementarity-determining region 3 (HC-CDR3).

17. The method of any of the preceding claims, wherein the antigenic target is a soluble protein antigen, a transmembrane protein antigen, or a viral antigen, optionally an HIV antigen.

18. The method of any of the preceding claims, wherein the antibody is an FDA-approved therapeutic antibody.

19. The method of any of the preceding claims, wherein the antibody is 10-1074 or a variant thereof.

20. The method of any of the preceding claims, wherein the heavy chain is a VH sequence and the light chain is a VK sequence of 10-1074.

21. The method of any of the preceding claims, wherein the HDR template (HDRT) is comprised within a double-stranded DNA (dsDNA) vector.

22. The method of any of the preceding claims, wherein the HDR template (HDRT) is comprised within an adeno-associated viral (AAV) vector.

23. The method of claim 22, wherein the AAV vector is encapsidated in an AAV6 or AAV-DJ capsid.

24. The method of any of the preceding claims, wherein the guide RNA comprises a sequence of between 15 and 200 nucleotides that is complementary to the genomic locus.

25. A method, comprising:
contacting a mature B cell obtained from a wild-type murine subject with an HDR template comprising a first sequence encoding heavy chain variable genes of a human antibody and/or an HDR template comprising a second sequence encoding light chain variable genes of the human antibody, a Cas protein, and a guide RNA,
wherein the B cell comprises a genomic locus encoding a B cell receptor (BCR), and wherein the guide RNA comprises a sequence having complementarity to the genomic locus;
whereby the Cas protein introduces a double-strand DNA break immediately adjacent to at least one target site in the genomic locus; and
administering the B cell comprising the engineered BCR to the subject;
wherein the at least one target site is replaced with the first sequence and/or the second sequence through HDR, thereby generating an engineered BCR.

26. A method comprising:
contacting a mature B cell obtained from a mammalian subject with a first HDR template comprising a first sequence encoding a heavy chain of a human antibody and a second HDR template comprising a second sequence encoding a light chain of the human antibody, a Cas protein, and a guide RNA,
wherein the B cell comprises a genomic locus encoding a B cell receptor (BCR), and wherein the guide RNA comprises a sequence having complementarity to the genomic locus;
whereby the Cas protein introduces a double-strand DNA break immediately adjacent to a first target site in the genomic locus and a second target site in the genomic locus; and
administering the B cell comprising the engineered BCR to the subject;
wherein the first target site is replaced with the first sequence, and the second target site is replaced with the second sequence, thereby generating an engineered BCR.

27. The method of claim 26, wherein the first target site is a VH sequence and the light chain is a VK sequence.

28. An affinity-matured antibody variant generated using the method of any of the preceding claims.

29. The antibody variant of claim 28, wherein the variant comprises an amino acid sequence having at least 85%, 90%, 92.5%, 95%, 98%, or 99% identity to any of SEQ ID NOs: 16-21.

30. The antibody variant of claim 28 or 29, wherein the variant comprises the amino acid sequence of any of SEQ ID NOs: 16-21.

31. An engineered mature B cell generated using the method of any of the preceding claims.

32. A B cell comprising the antibody variant of any one of claims 28-30.

33. A population of B cells in accordance with claim 32.

34. A method of administering the antibody variant of any one of claims 28-30, or the engineered B cell of any one of claims 31-33, to a subject.

35. The method of claim 34, wherein the subject is a human.

36. A system of affinity maturation of an antibody, comprising:
a B cell obtained from a mammalian subject with a homology-directed repair (HDR) template comprising a first sequence encoding a heavy chain of an antibody and/or an HDR template comprising a second sequence encoding a light chain, wherein the B cell comprises a genomic locus encoding a B cell receptor (BCR),
a Cas protein,
a guide RNA, wherein the guide RNA comprises a sequence having complementarity to the genomic locus, and
an injection mechanism for administering the B cell comprising the engineered BCR to the subject.

37. The system of claim 36, wherein the injection mechanism is adapted for subcutaneous or intraperitoneal injection.

38. A nucleic acid molecule encoding an engineered murine B cell receptor (BCR) comprising a VH domain derived from a human antibody, and a VK domain derived from a human antibody,
wherein the human antibody recognizes an HIV antigen, and
wherein the nucleic acid molecule comprises endogenous murine BCR regulatory elements.

39. The nucleic acid molecule of claim 38, wherein the human antibody is 10-1074, or a variant thereof.

40. A pharmaceutical composition comprising the antibody variant of any one of claims 28-30, or the engineered B cell of any one of claims 31-33.
